(19) United States
(12) Patent Application Publication
(10) Pub. No.: US 2022/0175762 A1
(43) Pub. Date: Jun. 9, 2022

(54) IMMUNE MODULATORY COMPOSITIONS AND METHODS FOR TREATING CANCERS

(71) Applicant: Birdie Biopharmaceuticals, Inc., Grand Cayman (KY)

(72) Inventor: Lixin Li, Beijing (CN)

(21) Appl. No.: 17/439,333

(22) PCT Filed: Mar. 13, 2020
(86) PCT No.: PCT/US20/22575

§ 371 Date: Sep. 14, 2021

Related U.S. Application Data
(60) Provisional application No. 62/818,942, filed on Mar. 15, 2019.

(51) Int. Cl. A61K 31/4745; A61K 31/69; A61K 31/282; A61K 31/675; A61K 31/506; A61K 31/495; A61K 31/337; A61K 31/513; A61K 31/7068; A61K 33/243; A61K 31/404; A61K 31/704; A61P 35/00

(52) U.S. Cl. CPC A61K 31/4745; A61K 31/69; A61K 31/282; A61K 31/675; A61K 31/506; A61K 31/495; A61P 35/00; A61K 31/513; A61K 31/7068; A61K 33/243; A61K 31/404; A61K 31/704; A61K 31/337

(57) ABSTRACT

The present disclosure relates to immune modulatory compositions and methods for treating cancers using combination therapy.

### CROSS REFERENCE TO RELATED APPLICATIONS

This application claims the benefit of U.S. Provisional Application No. 62/818,942, filed on Mar. 15, 2019, the entire contents of which are incorporate by reference herein.

### FIELD OF THE INVENTION

The present disclosure relates to immune modulatory compositions and methods for treating cancers using combination therapy.

### BACKGROUND

In recent years, low dose cyclophosphamide has been used clinically either as a single agent or as combination therapy to treat acute myeloid leukemia (AML), breast cancer, Chronic lymphocytic leukemia (CLL), Chronic myelogenous leukemia (CML), Hodgkin lymphoma, multiple myeloma, mycosis fungoides, neuroblastoma, Non-Hodgkin lymphoma (NHL), ovarian cancer, and retinoblastoma.

The efficacy of low dose cyclophosphamide is primarily due to its ability to immune-modulate in addition to its direct anti-tumor effects. The effect on regulatory T cells (Treg) is quite relevant for low dose chemotherapeutic treatments. Treg are CD4⁺ CD25⁺ Foxp3⁺ lymphocytes that can inhibit antigen-specific immune responses both in a cytokine dependent and cell contact dependent manner. Treg can thus inhibit antitumor immune response by suppressing the activity of both tumor specific (CD8⁺ cytotoxic T lymphocytes and CD4⁺ T helper cells) and tumor unspecific effector cells (natural killer (NK) and NK T cells). In a variety of human cancers, Treg cells have been found in increased proportions which can correlate with tumor progression and lack of treatment response. So, impairment of Treg activity by either specific blockade or depletion is a method to enhance immune response against tumor associated antigens. Many studies (preclinical and clinical) have documented the effect of low dose cyclophosphamide on Treg cells. It reduces the number of Treg cells, suppresses the function of the Treg cells and increases both lymphocyte proliferation and memory T cells. This ability highlights the potential for synergism between conventional chemotherapy and novel immunotherapy.

Toll-like receptors (TLRs) are a crucial part of the innate immunity and present the first line of defense against pathogens. Resiquimod is a ligand for TLR7 and TLR8 and directly activates innate immune cells, including myeloid dendritic cells, plasmacytoid dendritic cells, and monocytes/macrophages. This activation may result in activation of co-stimulatory molecules, production of antiviral cytokines, and stimulation of cell-mediated NK and T cell immune responses.

### SUMMARY

In general, the present disclosure provides therapeutic combinations and methods for treatment of cancers.

In one aspect, the present disclosure provides a combination, comprising: an effective amount of an immune modulatory chemotherapeutic; and an effective amount of immunotherapeutic comprising a TLR7 and/or TLR8 agonist activity. That is, the immunotherapeutic is an agonist for TLR7 or TLR8 or both.

In some embodiments, the immunotherapeutic has a structure of Formula (I):

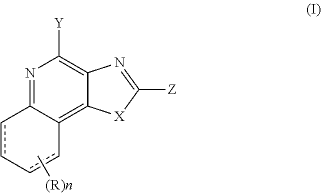

wherein dashed line represents bond or absence of bond;
X is S or —NR₁, R₁ is —W₀—W₁—W₂—W₃—W₄,
W₀ is a bond, alkyl, alkenyl, alkynyl, alkoxy, or -alkyl-S-alkyl-,
W₁ is a bond, —O—, or —NR₂—, wherein R₂ is hydrogen, alkyl or alkenyl,
W₂ is a bond, —O—, —C(O)—, —C(S)—, or —S(O)₂—,
W₃ is a bond, —NR₃—, wherein R₃ is hydrogen, alkyl or alkenyl,
W₄ is hydrogen, alkyl, alkenyl, alkynyl, alkoxy, cycloalkyl, aryl, aryloxy, heteroaryl, or heterocyclyl, each of which is optionally substituted by one or more substituents selected from the group consisting of hydroxyl, alkoxy, alkyl, alkenyl, alkynyl, cycloalkyl, aryl, heteroaryl, heterocyclyl, —NH₂, nitro, -alkyl-hydroxyl, -alkyl-aryl, -alkyl-heteroaryl, -alkyl-heterocyclyl, —O—R₄, —O-alkyl-R₄, -alkyl-O—R₄, —C(O)—R₄, -alkyl-C(O)—R₄, -alkyl-C(O)—O—R₄, —C(O)—O—R₄, —S—R₄, —S(O)₂—R₄, —NH—S(O)₂—R₄, -alkyl-S—R4, -alkyl-S(O)₂—R₄, —NHR₄, —NR₄R₄, —NH-alkyl-R₄, halogen, —CN, —NO₂, and —SH, wherein R₄ is independently hydrogen, alkyl, alkenyl, -alkyl-hydroxyl, aryl, heteroaryl, heterocyclyl, or haloalkyl;
is hydrogen, alkyl, alkenyl, alkynyl, alkoxy, aryl, haloalkyl, heteroaryl, heterocyclyl, each of which can be optionally substituted by one or more substituents selected from the group consisting of hydroxyl, alkoxy, alkyl, alkenyl, alkynyl, aryl, heteroaryl, heterocyclyl, halogen, cyano, nitro, —N(R₅)₂, -alkoxy-alkyl,
-alkoxy-alkenyl, —C(O)-alkyl, —C(O)—O-alkyl, —O—C(O)-alkyl, —C(O)—N(R₅)₂, aryl, heteroaryl, —CO-aryl, and —CO-heteroaryl, wherein each R₅ is independently hydrogen, alkyl, haloalkyl, -alkyl-aryl, or -alkyl-heteroaryl;
R is hydrogen, alkyl, alkoxy, haloalkyl, halogen, aryl, heteroaryl, heterocyclyl, each of which is optionally substituted by one or more substituents selected from the group consisting of hydroxyl, alkoxy, alkyl, alkenyl, alkynyl, cycloalkyl, aryl, heteroaryl, heterocyclyl, —NH₂, nitro, -alkyl-hydroxyl, -alkyl-aryl,
-alkyl-heteroaryl, -alkyl-heterocyclyl, —O—R₄, —O-alkyl-R₄, -alkyl-O—R₄, —C(O)—R₄, —C(O)—NH—R₄, —C(O)—NR₄R₄, -alkyl-C(O)—R₄, -alkyl-C(O)—O—R₄, —C(O)—O—R₄, —O—C(O)—R₄, —S—R₄, —C(O)—S—R₄, —S—C(O)—R₄,
—S(O)₂—R₄, —NH—S(O)₂—R₄, -alkyl-S—R₄, -alkyl-S(O)₂—R₄, —NHR₄, —NR₄R₄, —NH-alkyl-R₄, halogen, —CN, and —SH, wherein R₄ is independently hydrogen, alkyl, alkenyl, alkoxy, -alkyl-hydroxyl, aryl, heteroaryl, heterocyclyl, or haloalkyl;
n is 0, 1, 2, 3, or 4;
Y is —NR₆R₇, —CR₆R₇R₈, or -alkyl-NH₂, each of which can be optionally substituted by one or more substituents selected from the group consisting of hydroxyl, alkoxy, alkyl, alkenyl, alkynyl, —NH₂, halogen, —N(R₅)₂, -alkoxy-alkyl, -alkoxy-alkenyl, —C(O)-alkyl, —C(O)—O-alkyl, —C(O)—N(R₅)₂, aryl, heteroaryl, —CO-aryl, and —CO-heteroaryl,
wherein R₆, R₇ and R₈ are independently hydrogen, alkyl, alkenyl, alkoxy, alkylamino, dialkylamino, alkylthio, arylthio, -alkyl-hydroxyl, -alkyl-C(O)—O—R₉, -alkyl-C(O)—R₉, or -alkyl-O—C(O)—R₉, wherein each R₅ is independently hydrogen, alkyl, haloalkyl, -alkyl-aryl, or -alkyl-heteroaryl, wherein R₉ is hydrogen, alkyl, alkenyl, halogen, or haloalkyl;
X and Z taken together may optionally form a (5-9)-membered ring.

In some embodiments, the immunotherapeutic is a compound selected from the group consisting of: 2-propylthiazolo[4,5-c]quinolin-4-amine,
1-(2-methylpropyl)-1H-imidazo[4,5-c]quinolin-4-amine,
4-amino-2-(ethoxymethyl)-aa-di-methyl-1H-imidazo[4,5-c]quinoline-1-ethanol,
1-(4-amino-2-ethylaminomethylimidazo-[4,5-c]quinolin-1-yl)-2-methylpropan-2-ol,
N-[4-(4-amino-2-ethyl-1H-imidazo[4,5-c]quinolin-1-yl)butyl-]methanesulfonamide,
4-amino-2-ethoxymethyl-aa-dimethyl-6,7,8,9-tetrahydro-1h-imidazo[4,5-c]quinoline-1-ethanol,
4-amino-aa-dimethyl-2-methoxyethyl-1h-imidazo[4,5-c]quinoline-1-ethanol,
1-{2-[3-(benzyloxy)propoxy]ethyl}-2-(ethoxymethyl)-1H-imidazo[4,5-c]quinolin-4-amine,
1-(2-amino-2-methylpropyl)-2-(ethoxymethyl)-1H-imidazo[4,5-c]quinolin-4-amine,
1-{4-[(3,5-dichlorophenyl)sulfonyl]butyl}-2-ethyl-1H-imidazo[4,5-c]quinolin-4-amine,
N-{3-[4-amino-2-(ethoxymethyl)-1H-imidazo[4,5-c]quinolin-1-yl]propyl}-n′-(3-cyanophenyl)thiourea, N-[3-(4-amino-2-butyl-1H-imidazo[4,5-c]quinolin-1-yl)-2,2-dimethylpropyl]benzamide,
2-butyl-1-[3-(methylsulfonyl)propyl]-1H-imidazo[4,5-c]quinolin-4-amine,
N-{2-[4-amino-2-(ethoxymethyl)-1H-imidazo[4,5-c]quinolin-1-yl]-1,1-dimethylethyl}-2-ethoxyacetamide,
1-[4-amino-2-ethoxymethyl-7-(pyridin-4-yl)-1H-imidazo[4,5-c]quinolin-1-yl]-2-methylpropan-2-ol,
1-[4-amino-2-(ethoxymethyl)-7-(pyridin-3-yl)-1H-imidazo[4,5-c]quinolin-1-yl]-2-methylpropan-2-ol,
N-{3-[4-amino-1-(2-hydroxy-2-methylpropyl)-2-(methoxyethyl)-1H-imidazo[4,5-c]quinolin-7-yl]phenyl}methanesulfonamide,
1-[4-amino-7-(5-hydroxymethylpyridin-3-yl)-2-(2-methoxyethyl)-1H-imidazo[4,5-c]quinolin-1-yl]-2-methylpropan-2-ol,
3-[4-amino-2-(ethoxymethyl)-7-(pyridin-3-yl)-1H-imidazo[4,5-c]quinolin-1-yl]propane-1,2-diol,
1-[2-(4-amino-2-ethoxymethyl-1H-imidazo[4,5-c]quinolin-1-yl)-1,1-dimethylethyl]-3-propylurea,
1-[2-(4-amino-2-ethoxymethyl-1H-imidazo[4,5-c]quinolin-1-yl)-1,1-dimethylethyl]-3-cyclopentylurea,
1-[(2,2-dimethyl-1,3-dioxolan-4-yl)methyl]-2-(ethoxymethyl)-7-(4-hydroxymethylphenyl)-1H-imidazo[4,5-c]quinolin-4-amine,
4-[4-amino-2-ethoxymethyl-1-(2-hydroxy-2-methylpropyl)-1H-imidazo[4,5-c]quinolin-7-yl]-N-methoxy-N-methylbenzamide,
2-ethoxymethyl-N1-isopropyl-6,7,8,9-tetrahydro-1H-imidazo[4,5-c]quinoline-1,4-diamine,
1-[4-amino-2-ethyl-7-(pyridin-4-yl)-1H-imidazo[4,5-c]quinolin-1-yl]-2-methylpropan-2-ol, and
N-[4-(4-amino-2-ethyl-1H-imidazo[4,5-c]quinolin-1-yl)butyl]methanesulfonamide.

In some embodiments, the immunotherapeutic comprises resiquimod.

In some embodiments, the immunotherapeutic is of an amount that is capable of: (1) inducing IFN-α in an enriched human blood DCs; (2) inducing TNF-α in an enriched human blood DCs; and/or (3) inducing IL-12-α in an enriched human blood DCs.

In some embodiments, the immune modulatory chemotherapeutic comprises an anti-tumor agent, while other embodiments do not. In some embodiments, the anti-tumor agent is selected from the group consisting of: Anthracyclines, Bortezomib, Oxaliplatin, and Cyclophosphamide. Some embodiments specifically include one or more such agents, while other embodiments specifically exclude one or more or all such agents.

In some embodiments, the immune modulatory chemotherapeutic comprises a Treg inhibitor, while other embodiments do not. In some embodiments, the Treg inhibitor is selected from the group consisting of: Dasatinib, Cyclophoshamide, Temozolomide, Docetaxel, and 5-Fluorouracile. Some embodiments specifically include one or more such agents, while other embodiments specifically exclude one or more or all such agents.

In some embodiments, wherein the immune modulatory chemotherapeutic comprise a myeloid-derived suppressor cells (MDSC) inhibitor, while other embodiments do not. In some embodiments, the MDSC inhibitor is selected from the group consisting of: Paclitaxel, Gemcitabine, 5-Fluorouracile, Oxaliplatin, Cisplatin, Carboplatin, Dasatinib, Sunitinib, and Doxorubicin. Some embodiments specifically include one or more such agents, while other embodiments specifically exclude one or more or all such agents.

In some embodiments, the immune modulatory chemotherapeutic comprise an NK cell activator, such as Dasatinib, and Imatinib, while other embodiments do not. Some embodiments specifically include one or more such agents, while other embodiments specifically exclude one or more or all such agents.

In some embodiments, the combination is formulated for systematic delivery. In some embodiments, the combination is formulated for oral administration or parenteral injection. In some embodiments, the combination is formulated for intravenous injection or intratumoral injection.

In some embodiments, the immunotherapeutic is an agonist for both TLR7 and TLR8.

In another aspect, the present disclosure provides a method for treating tumor or abnormal cell proliferation, in a subject that is in need of such treatment, comprising administering to the subject the combination provided herein.

In some embodiments, the abnormal cell proliferation comprises a pre-cancerous lesion.

In some embodiments, the abnormal proliferation is of cancer cells. In some embodiments, the cancer is selected from the group consisting of: Acute myeloid leukemia (AML), Breast cancer, Chronic lymphocytic leukemia (CLL), Chronic myelogenous leukemia (CML), Hodgkin lymphoma, Multiple myeloma, Mycosis fungoides, Neuroblastoma, Non-Hodgkin lymphoma (NHL), Ovarian cancer, and Retinoblastoma.

In some embodiments, the method comprises administering to the subject an oral formulation comprising the immunotherapeutic in a dose of from about 0.0005 mg/kg, 0.0006 mg/mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, or 0.01 mg/kg, to about 0.02 mg/kg, all inclusive, twice per week. In further embodiments the dose is in a range bounded by any pair of doses in the preceeding list.

In some embodiments, the method comprises administering to the subject an oral formulation comprising the immunotherapeutic in a dose of at least 0.0001 mg/kg but less than or about 0.0005 mg/kg, 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, or 0.01 mg/kg, twice per week.

In some embodiments, the method comprises administering to the subject an intravenous formulation comprising the immunotherapeutic in a dose of from about 0.0005 mg/kg, 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, or 0.006 mg/kg to about 0.015 mg/kg, all inclusive, weekly. In further embodiments the dose is in a range bounded by any pair of doses in the preceeding list.

In some embodiments, the method comprises administering to the subject an intravenous formulation comprising the immunotherapeutic in a dose of at least 0.0001 mg/kg but less than or about 0.003 mg/kg, 0.004 mg/kg, 0.005 mg/kg, 0.006 mg/kg, or 0.01 mg/kg, weekly.

In some embodiments, the immunotherapeutic in the subject has a local concentration that is between about 0.005 μg/ml and about 12 μg/ml.

In some embodiments, the immunotherapeutic in the subject has a local concentration that is is from about 0.05 μg/ml, 0.1 μg/ml, 0.15 μg/ml, 0.2 μg/ml, 0.3 μg/ml, or 0.4 μg/ml, to about 0.5 μg/ml. In further embodiments the concentration is in a range bounded by any pair of doses in the preceeding list.

In some embodiments, the method comprises administering to the subject an intravenous formulation comprising the immune modulatory chemotherapeutic in a dose of about 40-50 mg/kg in divided dose over 2-5 days.

In some embodiments, wherein the combination is adminstered over 1-5 days repeatedly at intervals of 2-4 weeks.

In some embodiments, the method comprises administering to the subject an intravenous formulation comprising the immune modulatory chemotherapeutic in a dose of about 10 to 15 mg/kg, given every 7 to 10 days.

In some embodiments, the method comprises administering to the subject an intravenous formulation comprising the immune modulatory chemotherapeutic in a dose of about 3 to 5 mg/kg, twice weekly.

In some embodiments, the method comprises administering to the subject an intravenous formulation comprising the immune modulatory chemotherapeutic in a dose of about 60-120 mg/m²/day, continuous daily.

In some embodiments, the method comprises administering to the subject an oral formulation comprising the immune modulatory chemotherapeutic in a dose of about 400-1000 mg/m² divided over 4-5 days. In some embodiments such administration is repeated at intervals of 2-4 weeks.

In some embodiments, the method comprises administering to the subject an intravenous formulation comprising the immune modulatory chemotherapeutic in a dose of about 50-100 mg/m²/day, or 1-5 mg/kg/day. In some embodiments such administration is repeated at intervals of 2-4 weeks.

In a further aspect, the present disclosure provides a kit that contains the therapeutic combination provided herein, and optionally with an instruction.

### BRIEF DESCRIPTION OF THE DRAWINGS

The novel features of the present embodiments are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-D depicts evaluation of the antitumor effects of Cyclophosphamide/TLRL combination therapy in syngeneic tumor model. FIG. 1A: Scheme of therapy protocol developed to treat C26 tumor. The treatment was initiated at d10 with tumor size 100-120 mm³ following tumor challenges, and was administered weekly for 3 cycles starting at day 10. FIG. 1B: Antitumor effects of systemic TLRL (Resiquimod) in C26 tumor model. 4×10⁵ cells were inoculated subcutaneously in Balb/c mice. After the tumor was allowed to grow for 10 days, groups of 8 mice were treated with Vehicle or with intraperitoneally administration of 1 mg of Cyclophosphamide, or with intravenously administered 1.6 g of TLRL or combination therapy at the dose shown. The compound was administered weekly for three cycles. The data are represented by the median tumor volume measurements of each group. Data for each group of 8 mice are expressed as means±SD. Statistical comparisons were performed using Student's t-Test. FIG. 1C depicts median tumor volume measurements during treatment with 1.6 g of resiquimod i.v. with low dose of cyclophosphamide i.p. weekly. C26 tumor growth curves of vehicle, 1 mg of Cyclophosphamide, 1.6 g of TLRL (Resiquimod), or 1.6 g of TLRL (Resiquimod) with various doses of Cyclophosphamide at the dose shown, in Balb/c mice. The compound was administered weekly for three cycles. These data are represent by the median tumor volume measurements for each group (n=8). FIG. 1D depicts median tumor volume measurements during treatment with 3.2 μg of resiquimod i.v. with low dose of cyclophosphamide i.p. weekly C26. Tumor growth curves of vehicles, 1 mg of Cyclophosphamide, 3.2 g of TLRL (Resiquimod), or 3.2 g of TLRL (Resiquimod) with various dose of Cyclophosphamide at the dose shown in Balb/c mice. The compound was administered weekly for three times. These data represent the median tumor volume measurements for each group (n=8).

FIG. 2 depicts increased infiltration of activated immune cells (CD45⁺) in TLRL or TLRL/Cyc treated tumor. Histological characterization of CD45 positive cells were performed in treated mice. Tumor samples were obtained from mice treated with TLRL, cyclophosphamide or TLRL/cyclophosphamide. Histological sections were counterstained with hematoxylin and eosin (H&E). CD45 protein was visualized by darker (original:brown) color, some of which is indicated by arrows.

FIG. 3 depicts increased IFNα inducible gene expression in TLRL or TLRL/Cyc treated tumor. Expression of IFNα inducible genes in dLN and spleen of treated mouse. RNA was isolated from draining lymph nodes and spleen after second treatment with TLRL or cyclophosphamide or combination therapy and relative expression of IFNα inducible genes MX2, ISG15 and IRF7 were determined by quantitative RT-PCR. Values indicate the mRNA expression of indicated IFNα inducible genes relative to housekeeping gene Actin.

### DETAILED DESCRIPTION

Several aspects of the present embodiments are described below with reference to example applications for illustration. It should be understood that numerous specific details, relationships, and methods are set forth to aide in attaining a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events.

Furthermore, not all illustrated acts or events are required to implement a methodology in accordance with the present embodiments.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

### I. Definitions and Abbreviations

Unless defined otherwise, all technical and scientific terms used herein generally have the same meaning as commonly understood by one of ordinary skill in the art to which the present embodiments belong. Generally, the nomenclature used herein and the laboratory procedures in cell culture, molecular genetics, organic chemistry and nucleic acid chemistry and hybridization are those well-known and commonly employed in the art. Standard techniques are used for nucleic acid and peptide synthesis. The techniques and procedures are generally performed according to conventional methods in the art and various general references, which are provided throughout this document. The nomenclature used herein and the laboratory procedures in analytical chemistry, and organic synthetic described below are those well-known and commonly employed in the art. Standard techniques, or modifications thereof, are used for chemical syntheses and chemical analyses.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups, which are limited to hydrocarbon groups, are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present embodiments. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

In general, an “acyl substituent” is also selected from the group set forth above. As used herein, the term “acyl substituent” refers to groups attached to, and fulfilling the valence of a carbonyl carbon that is either directly or indirectly attached to the polycyclic nucleus of the compounds of the present embodiments.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

As used herein, the term “haloalkyl” refers to an alkyl as defined herein, that is substituted by one or more halo groups as defined herein. Preferably the haloalkyl can be monohaloalkyl, dihaloalkyl or polyhaloalkyl including perhaloalkyl. A monohaloalkyl can have one iodo, bromo, chloro or fluoro within the alkyl group. Dihaloalkyl and polyhaloalkyl groups can have two or more of the same halo atoms or a combination of different halo groups within the alkyl. Preferably, the polyhaloalkyl contains up to 12, 10, or 8, or 6, or 4, or 3, or 2 halo groups. Non-limiting examples of haloalkyl include fluoromethyl, difluoromethyl, trifluoromethyl, chloromethyl, dichioromethyl, trichioromethyl, pentafluoroethyl, heptafluoropropyl, difluorochioromethyl, dichiorofluoromethyl, difluoroethyl, difluoropropyl, dichloroethyl and dichioropropyl. A perhaloalkyl refers to an alkyl having all hydrogen atoms replaced with halo atoms.

As used herein, the term “heteroaryl” refers to a 5-14 membered monocyclic- or bicyclic- or fused polycyclic-ring system, having 1 to 8 heteroatoms selected from N, O, S or Se. Preferably, the heteroaryl is a 5-10 membered ring system. Typical heteroaryl groups include 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2, 3-triazolyl, tetrazolyl, 2-, 3-, or 4-pyridyl, 3- or 4-pyridazinyl, 3-, 4-, or 5-pyrazinyl, 2-pyrazinyl, 2-, 4-, or 5-pyrimidinyl.

The term “heteroaryl” also refers to a group in which a heteroaromatic ring is fused to one or more aryl, cycloaliphatic, or heterocycloalkyl rings, where the radical or point of attachment is on the heteroaromatic ring. Nonlimiting examples include but are not limited to 1-, 2-, 3-, 5-, 6-, 7-, or 8-indolizinyl, 1-, 3-, 4-, 5-, 6-, or 7-isoindolyl, 2-, 3-, 4-, 5-, 6-, or 7-indolyl, 2-, 3-, 4-, 5-, 6-, or 7-indazolyl, 2-, 4-, 5-, 6-, 7-, or 8-purinyl, 1-, 2-, 3-, 4-, 6-, 7-, 8-, or 9-quinolizinyl, 2-, 3-, 4-, 5-, 6-, 7-, or 8-quinoliyl, 1-, 3-, 4-, 5-, 6-, 7-, or 8-isoquinoliyl, 1-, 4-, 5-, 6-, 7-, or 8-phthalazinyl, 2-, 3-, 4-, 5-, or 6-naphthyridinyl, 2-, 3-, 5-, 6-, 7-, or 8-quinazolinyl, 3-, 4-, 5-, 6-, 7-, or 8-cinnolinyl, 2-, 4-, 6-, or 7-pteridinyl, 1-, 2-, 3-, 4-, 5-, 6-, 7-, or 8-4aH carbazolyl, 1-, 2-, 3-, 4-, 5-, 6-, 7-, or 8-carbzaolyl, 1-, 3-, 4-, 5-, 6-, 7-, 8-, or 9-carbolinyl, 1-, 2-, 3-, 4-, 6-, 7-, 8-, 9-, or 10-phenanthridinyl, 1-, 2-, 3-, 4-, 5-, 6-, 7-, 8-, or 9-acridinyl, 1-, 2-, 4-, 5-, 6-, 7-, 8-, or 9-perimidinyl, 2-, 3-, 4-, 5-, 6-, 8-, 9-, or 10-phenathrolinyl, 1-, 2-, 3-, 4-, 6-, 7-, 8-, or 9-phenazinyl, 1-, 2-, 3-, 4-, 6-, 7-, 8-, 9-, or 10-phenothiazinyl, 1-, 2-, 3-, 4-, 6-, 7-, 8-, 9-, or 10-phenoxazinyl, 2-, 3-, 4-, 5-, 6-, or 1-, 3-, 4-, 5-, 6-, 7-, 8-, 9-, or 10-benzisoqinolinyl, 2-, 3-, 4-, or 5-thieno[2,3-b]furanyl, 2-, 3-, 5-, 6-, 7-, 8-, 9-, 10-, or 11-7H-pyrazino[2,3-c]carbazolyl, 2-, 3-, 5-, 6-, or 7-2H-furo[3,2-b]-pyranyl, 2-, 3-, 4-, 5-, 7-, or 8-5H-pyrido[2,3-d]-o-oxazinyl, 1-, 3-, or 5-1H-pyrazolo[4,3-d]-oxazolyl, 2-, 4-, or 54H-imidazo[4,5-d]thiazolyl, 3-, 5-, or 8-pyrazino[2,3-d]pyridazinyl, 2-, 3-, 5-, or 6-imidazo[2,1-b] thiazolyl, 1-, 3-, 6-, 7-, 8-, or 9-furo[3,4-c]cinnolinyl, 1-, 2-, 3-, 4-, 5-, 6-, 8-, 9-, 10, or 11-4H-pyrido[2,3-c]carbazolyl, 2-, 3-, 6-, or 7-imidazo[1,2-b][1,2,4]triazinyl, 7-benzo[b]thienyl, 2-, 4-, 5-, 6-, or 7-benzoxazolyl, 2-, 4-, 5-, 6-, or 7-benzimidazolyl, 2-, 4-, 4-, 5-, 6-, or 7-benzothiazolyl, 1-, 2-, 4-, 5-, 6-, 7-, 8-, or 9-benzoxapinyl, 2-, 4-, 5-, 6-, 7-, or 8-benzoxazinyl, 1-, 2-, 3-, 5-, 6-, 7-, 8-, 9-, 10-, or 11-1H-pyrrolo[1,2-b][2]benzazapinyl. Typical fused heteroaryl groups include, but are not limited to 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl.

As used herein, the term “heterocyclyl” or “heterocyclo” refers to an optionally substituted, fully saturated or unsaturated, aromatic or nonaromatic cyclic group, e.g., which is a 4- to 7-membered monocyclic, 7- to 12-membered bicyclic or 10- to 15-membered tricyclic ring system, which has at least one heteroatom in at least one carbon atom-containing ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized. The heterocyclic group may be attached at a heteroatom or a carbon atom.

Exemplary monocyclic heterocyclic groups include pyrrolidinyl, pyrrolyl, pyrazolyl, oxetanyl, pyrazolinyl, imidazolyl, imidazolinyl, imidazolidinyl, triazolyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, 1,1,4-trioxo-1,2,5-thiadiazolidin-2-yl and the like.

Exemplary bicyclic heterocyclic groups include indolyl, dihydroidolyl, benzothiazolyl, benzoxazinyl, benzoxazolyl, benzothienyl, benzothiazinyl, quinuclidinyl, quinolinyl, tetrahydroquinolinyl, decahydroquinolinyl, isoquinolinyl, tetrahydroisoquinolinyl, decahydroisoquinolinyl, benzimidazolyl, benzopyranyl, indolizinyl, benzofuryl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]-pyridinyl] or furo[2,3-b]pyridinyl), dihydroisoindolyl, 1,3-dioxo-1,3-dihydroisoindol-2-yl, dihydroquinazolinyl (such as 3,4-dihydro-4-oxo-quinazolinyl), phthalazinyl and the like.

Exemplary tricyclic heterocyclic groups include carbazolyl, dibenzoazepinyl, dithienoazepinyl, benzindolyl, phenanthrolinyl, acridinyl, phenanthridinyl, phenoxazinyl, phenothiazinyl, xanthenyl, carbolinyl and the like.

The term “heterocyclyl” further refers to heterocyclic groups as defined herein substituted with 1, 2 or 3 substituents selected from the groups consisting of the following:

(a) alkyl;

(b) hydroxy (or protected hydroxy);

(c) halo;

(d) oxo, i.e., ═O;

(e) amino, alkylamino or dialkylamino;

(f) alkoxy;

(g) cycloalkyl;

(h) carboxy;

(i) heterocyclooxy, wherein heterocyclooxy denotes a heterocyclic group bonded through an oxygen bridge;

(j) alkyl-O—C(O)—;

(k) mercapto;

(l) nitro;

(m) cyano;

(n) sulfamoyl or sulfonamido;

(o) aryl;

(p) alkyl-C(O)—O—;

(q) aryl-C(O)—O—;

(r) aryl-S—;

(s) aryloxy;

(t) alkyl-S—;

(u) formyl, i.e., HC(O)—;

(v) carbamoyl;

(w) aryl-alkyl-; and

(x) aryl substituted with alkyl, cycloalkyl, alkoxy, hydroxy, amino, alkyl-C(O)—NH—, alkylamino, dialkylamino or halogen.

As used herein, the term “alkenyl” refers to a straight or branched hydrocarbon group having 2 to 20 carbon atoms and that contains at least one double bonds. The alkenyl groups preferably have about 2 to 8 carbon atoms.

The term “aryl” means, unless otherwise stated, a polyunsaturated, aromatic, hydrocarbon substituent, which can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) include both substituted and unsubstituted forms of the indicated radical. Preferred substituents for each type of radical are provided below.

Substituents for the alkyl, and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generally referred to as “alkyl substituents” and “heteroakyl substituents,” respectively, and they can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the present embodiments includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, the aryl substituents and heteroaryl substituents are generally referred to as “aryl substituents” and “heteroaryl substituents,” respectively and are varied and selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the present embodiments includes more than one R group, for example, each of the R groups is independently selected as are each R′, R″, R′″ and R″″ groups when more than one of these groups is present.

Two of the aryl substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆) akyl.

As used herein, the term “heteroatom” includes oxygen (O), nitrogen (N), sulfur (S), phosphorus (P) and silicon (Si).

As used herein, the term “aryloxy” refers to both an —O-aryl and an —O-heteroaryl group, wherein aryl and heteroaryl are defined herein.

As used herein, the term “pharmaceutically acceptable salts” refers to salts that retain the biological effectiveness and properties of the compounds of the present embodiments and, which are not biologically or otherwise undesirable. In many cases, the compounds of the present embodiments are capable of forming acid and/or base salts by virtue of the presence of amino and/or carboxyl groups or groups similar thereto (e.g., phenol or hydroxyamic acid). Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. The pharmaceutically acceptable salts of compounds of the present embodiments can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts can be found, e.g., in Remington's Pharmaceutical Sciences, 20th ed., Mack Publishing Company, Easton, Pa., (1985), which is herein incorporated by reference.

As used herein, the term “pharmaceutically acceptable carrier/excipient” includes any and all solvents, dispersion media, coatings, surfactants, antioxidants, preservatives (e.g., antibacterial agents, antifungal agents), isotonic agents, absorption delaying agents, salts, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except in so far as any conventional carrier is incompatible with the active ingredient, its use in the therapeutic or pharmaceutical compositions is contemplated.

As used herein, the term “subject” refers to an animal. Preferably, the animal is a mammal. A subject also refers to for example, primates (e.g., humans), cows, sheep, goats, horses, dogs, cats, rabbits, rats, mice, fish, birds and the like. In a preferred embodiment, the subject is a human.

As used herein, the term “therapeutic combination” or “combination” refers to a combination of one or more active drug substances, i.e., compounds having a therapeutic utility. Typically, each such compound in the therapeutic combinations of the present embodiments will be present in a pharmaceutical composition comprising that compound and a pharmaceutically acceptable carrier. The compounds in a therapeutic combination of the present embodiments may be administered simultaneously or separately, as part of a regimen.

### II. Compositions

In general, the present disclosure provides therapeutic combinations, pharmaceutical compostions, and methods for treating cancers using combination therapy. More specifically, the combination of immunotherapy (such as using Toll-like Receptor Ligand “TLRL”), for example, a TLR7/8 agonist to activate DCs in innate immunity, with low dose of immune modulatory chemotherapy.

In one aspect, the present disclosure provides therapeutic combinations, or pharmaceutical compositions, comprising: (i) an effective amount of an immune modulatory chemotherapeutic; and (ii) an effective amount of immunotherapeutic comprising TLR7 and/or TLR8 agonist activity. That is, in various embodiments, the immunotherapeutic is an agonist for TLR7, or TLR8, or both.

A therapeutic combination may be provided in a single pharmaceutical composition so that both the immune modulatory chemotherapeutic and the immunotherapeutic can be administered together. In alternative embodiments, a therapeutic combination may be provided using more than one pharmaceutical composition. In such embodiments, an immune modulatory chemotherapeutic may be provided in one pharmaceutical composition and an immunotherapeutic may be provided in a second pharmaceutical composition so that the two compounds can be administered separately such as, for example, at different times, by different routes of administration, and the like. Thus, it also may be possible to provide the immune modulatory chemotherapeutic and the immunotherapeutic in different dosing regimens.

Unless otherwise indicated, reference to a compound can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers.

In general, the immune modulatory chemotherapeutic and the immunotherapeutic are not linked to each other, such as by a covalent linker.

### A. Immune Modulatory Chemotherapeutics

In general, the compostions provided herein comprise an immune modulatory chemotherapeutic.

By “immune modulatory chemotherapeutic” herein is meant a therapeutic agent that is able to reduce host regulatory T cells (Tregs) and/or MDSC subpopulations in addition to their direct anti-tumor effects.

In some embodiments, the immune modulatory chemotherapeutic comprises an anti-tumor agent.

By “anti-tumor agent” herein is meant a therapeutic agent that directly kills tumor cells by conventional cytotoxic antitumor activity. In some embodiments, the anti-tumor agent is selected from the group consisting of: Anthracyclines, Bortezomib, Oxaliplatin, and Cyclophosphamide.

In some embodiments, the immune modulatory chemotherapeutic comprises a Treg inhibitor.

By “Treg inhibitor” herein is meant a therapeutic agent that regulates CD25⁺/CD45⁺ Treg cells.

In some embodiments, the Treg inhibitor is selected from the group consisting of: Dasatinib, Cyclophoshamide, Temozolomide, Docetaxel, and 5-Fluorouracile.

In some embodiments, the immune modulatory chemotherapeutic comprises a myeloid-derived suppressor cells (MDSC) inhibitor.

By “myeloid-derived suppressor cells (MDSC) inhibitor” herein is meant a therapeutic agent that is able to regulate immune suppressive cells such as MDSC”. In some embodiments, the MDSC inhibitor is selected from the group consisting of: Paclitaxel, Gemcitabine, 5-Fluorouracile, Oxaliplatin, Cisplatin, Carboplatin, Dasatinib, Sunitinib, and Doxorubicin.

In some embodiments, the immune modulatory chemotherapeutic comprise an NK cell activator.

By “NK cell activator” herein is meant a therapeutic agent that is able to active NK cells in the tumor microenviorment. In some embodiments, the NK cell activator is selected from the group consisting of: Dasatinib and Imatinib.

### B. Immunotherapeutics

In general, the combination or compostions of the present disclosure comprise an immunotherapeutic.

By “immunotherapeutics” herein is meant a compound, a molecule, or an agent that is capable of stimulating or enhancing the body's immune system or tumor cells. Immunotherapetuics are used for the treatment of disease by inducing, enhancing, or suppressing an immune response. Immunotherapeutics of the present embodiments generally are designed to elicit or amplify an immune response, rather than suppress an immune response.

In general, the immunoethreapeutics of the present embodiments act, directly or indirectly, on toll like receptors, nucleotide-oligomerization domain-like receptors, RIG-I-Like receptors, c-type lectin receptors, or cytosolic DNA Sensors, or a combination thereof. Particularly, the immunotherapeutics of the present embodiments are capable of activating a human plasmacytoid dendritic cell, myeloid dendritic cell, NK cell, or tumor cell, or a combination thereof.

In some embodiments, the immunotherapeutics of the present embodiments activate human immune cells, including but not limited to dendritic cells, macrophages, monocytes, myeloid-derived suppressor cells, NK cells, B cells, T cells, or tumor cells, or a combination thereof.

Dendritic cells are the most powerful antigen-presenting cells. Dendritic cells play an essential role for the initiation of both innate and adaptive immune responses. Dendritic cells also play a key role in the induction and maintenance of immune tolerance.

By “dendritic cells” (DC) herein is meant a heterogeneous cell population including two main subtypes: namely, myeloid DC (mDC) and plasmacytoid DC (pDC) (Steinman et al., 1979, J. Exp. Med., 149, 1-16). These two blood DC subsets were originally differentiated by their expression of CD11c (integrin complement receptor) and CD123 (IL-3Rα). Each of the pDC and mDC populations constitutes between about 0.2 to about 0.6% of the PBMC population in humans.

By “pDC” herein is meant plasmacytoid dendritic cells and they represent a subtype of dendritic cells found in the blood and peripheral lymphoid organs. These cells express the surface markers CD123, BDCA-2(CD303) and BDCA-4(CD304) and HLA-DR, but do not express CD11c, CD14, CD3, CD20 or CD56, which distinguishes them from conventional dendritic cells, monocytes, T-cells, B cells and NK cells. As components of the innate immune system, these cells express intracellular Toll-like receptors 7 and 9, which enable the detection of viral and bacterial nucleic acids, such as ssRNA or CpG DNA motifs. Upon stimulation and subsequent activation, these cells produce large amounts of Type I interferon (mainly IFN-α and IFN-β) and Type III interferon (e.g., IFN-γ), which are critical pleiotropic anti-viral compounds mediating a wide range of effects. By generating a large number of type I interferon, cytokines and chemokines, plasmacytoid dendritic cells are widely involved in the body's innate and adaptive immune responses. They can regulate NK cells, T cells, B cells and other cells involved in immune response intensity, duration, and response mode, thus play a very important function in tumor, infection and autoimmune disease. (Liu Y J. IPC: professional type 1 interferon-producing cells and plasmacytoid dendritic cell precursors. Annu Rev Immunol. 2005; 23:275-306. Gilliet M, Cao W, Liu Y J. Plasmacytoid dendritic cells: sensing nucleic acids in viral infection and autoimmune diseases. Nat Rev Immunol. 2008 August; 8 (8):594-606).

By “mDC” herein is meant myeloid dendritic cells and they represent a subtype of circulating dendritic cells found in blood and peripheral lymphoid organs. These cells express the surface markers CD11c, CD1a, HLA-DR and either BDCA-1 (CD1c) or BDCA-3 (CD141). They do not express BDCA-2 or CD123, which distinguishes them from pDC. mDC also do not express CD3, CD20 or CD56. As components of the innate immune system, mDC express Toll-like receptors (TLR), including TLR2, 3, 4, 5, 6 and 8, which enable the detection of bacterial and viral components. Upon stimulation and subsequent activation, these cells are the most potent antigen presenting cells to activate antigen-specific CD4 as well as CD8 T cells. In addition, mDCs has the ability to produce large amounts of IL-12 and IL23, which is critical for the induction of Th1-mediated or Th17 cell-mediated immunity.

Study has found that many solid tumors, such as breast cancer, head and neck cancer, and ovarian cancer, have pDC's invasion (Treilleux I, Blay J Y, Bendriss-Vermare N et al. Dendritic cell infiltration and prognosis of early stage breast cancer. Clin Cancer Res 2004; 10:7466-7474. Hartmann E, Wollenberg B, Rothenfusser S et al. Identification and functional analysis of tumor-infiltrating plasmacytoid dendritic cells in head and neck cancer. Cancer Res 2003; 63:6478-6487. Zou W P, Machelon V, Coulomb-L'Hermin A, et al. Stromal-derived factor-1 in human tumors recruits and alters the function of plasmacytoid precursor dendritic cells. Nat Med 2001; 7:1339-1346) and factors secreted by tumor cells inhibit DC maturation. (Gabrilovich D I, Corak J, Ciernik I F et al. Decreased antigen presentation by dendritic cells in patients with breast cancer. Clin Cancer Res 1997; 3:483-490. Bell D, Chomarat P, Broyles D et al. In breast carcinoma tissue, immature dendritic cells reside within the tumor, whereas mature dendritic cells are located in peritumoral areas. J Exp Med 1999; 190:1417-1425. Menetrier-Caux C, Montmain G, Dieu M C et al. Inhibition of the differentiation of dendritic cells from CD34 (+) progenitors by tumor cells: role of interleukin-6 and macrophage colony-stimulating factor. Blood 1998; 92:4778-4791). These immature DC cells did not play a role in promoting anti-tumor immunity. By contrast, DCs within the tumor microenvironment promote tumor growth by inhibiting antitumor immunity and by promoting angiogenesis. There is evidence that Toll-like receptor 7 agonist Imiquimod, and Toll-like receptor 9 agonist CpG drugs can stimulate pDC within the tumor microenvironment to inhibit tumor development. (Dummer R, Urosevic M, Kempf W et al. Imiquimod in basal cell carcinoma: how does it work? Br J Dermatol 2003; 149:57-58. Miller R L, Gerster J F, Owens M L et al Imiquimod applied topically: a novel immune response modifier and new class of drug. Int J Immunopharmacol 1999; 21:1-14. Hofmann M A, Kors C, Audring H et al Phase 1 evaluation of intralesionally injected TLR9-agonist PF-3512676 in patients with basal cell carcinoma or metastatic melanoma. J Immunother 2008; 31:520-527).

Natural killer (NK) cells are a type of cytotoxic lymphocyte that constitutes a major component of the immune system. NK cells are a subset of peripheral blood lymphocytes defined by the expression of CD56 or CD 16 and the absence of the T cell receptor (CD3). They recognize and kill transformed cell lines without priming in an MHC-unrestricted fashion. NK cells play a major role in the rejection of tumors and cells infected by viruses. The process by which an NK cell recognizes a target cell and delivers a sufficient signal to trigger target lysis is determined by an array of inhibitory and activating receptors on the cell surface. NK discrimination of self from altered self involves inhibitory receptor recognition of MHC-I molecules and non-MHC ligands like CD48 and Clr-1b. NK recognition of infected or damaged cells (altered self) is coordinated through stress induced ligands (e.g., MICA, MICB, Rae1, H60, Mult1) or virally encoded ligands (e.g., m157, hemagluttinin) recognized by various activating receptors, including NKG2D, Ly49H and NKp46/Ncr1.

NK cells represent the predominant lymphoid cell in the peripheral blood for many months after allogeneic or autologous stem cell transplant and they have a primary role in immunity to pathogens during this period (Reittie et al (1989) Blood 73:1351-1358; Lowdell et al (1998) Bone Marrow Transplant 21:679-686). The role of NK cells in engraftment, graft-versus-host disease, anti-leukemia activity and post-transplant infection is reviewed in Lowdell (2003) Transfusion Medicine 13:399-404.

Human NK cells mediate the lysis of tumor cells and virus-infected cells via natural cytotoxicity and antibody-dependent cellular cytotoxicity (ADCC).

Human NK cells are controlled by positive and negative cytolytic signals. Negative (inhibitory) signals are transduced by C-lectin domain containing receptors CD94/NKG2A and by some Killer Immunoglobulin-like Receptors (KIRs). The regulation of NK lysis by inhibitory signals is known as the “missing self” hypothesis in which specific HLA-class I alleles expressed on the target cell surface ligate inhibitory receptors on NK cells. The down-regulation of HLA molecules on tumor cells and some virally infected cells (e.g. CMV) lowers this inhibition below a target threshold and the target cells may become susceptible to NK cell-mediated lysis if the target cells also carry NK-priming and activating molecules. TLR7, TLR8 or TLR9 agonists can activate both mDC and pDCs to produce type IIFNs and express costimulatory molecules such as GITR-ligand, which subsequently activate NK cells to produce IFN-γ and potently promote NK cell killing function.

Inhibitory receptors fall into two groups, those of the Ig-superfamily called Killer Immunoglobulin-like Receptors (KIRs) and those of the lectin family, the NKG2, which form dimers with CD94 at the cell surface. KIRs have a 2- or 3-domain extracellular structure and bind to HLA-A, -B or -C. The NKG2/CD94 complexes ligate HLA-E.

Inhibitory KIRs have up to 4 intracellular domains which contain ITIMs and the best characterized are KIR2DL1, KIR2DL2 and KIR2DL3 which are known to bind HLA-C molecules. KIR2DL2 and KIR2DL3 bind the group 1 HLA-C alleles while KIR2DL1 binds to group 2 alleles. Certain leukemia/lymphoma cells express both group 1 and 2 HLA-C alleles and are known to be resistant to NK-mediated cell lysis.

With regards to positive activating signals, ADCC is thought to be mediated via CD 16, and a number of triggering receptors responsible for natural cytotoxicity have been identified, including CD2, CD38, CD69, NKRP-I, CD40, B7-2, NK-TR, NKp46, NKp30 and NKp44. Several KIR molecules with short intracytoplasmic tails are also stimulatory. These KIRs (KIR2DS1, KIR2DS2 and KIR2DS4) are known to bind to HLA-C; their extracellular domains being identical to their related inhibitory KIRs. The activatory KIRs lack the ITIMs and instead associate with DAP 12 leading to NK cell activation. The mechanism of control of expression of inhibitory versus activatory KIRs remains unknown.

Several reports have described the expression of TLRs in mouse or human cancer or cancer cell lines. For example, TLR1 to TLR6 are expressed by colon, lung, prostate, and melanoma mouse tumor cell lines (Huang B, et al. Toll-like receptors on tumor cells facilitate evasion of immune surveillance. Cancer Res. 2005; 65(12):5009-5014.), TLR3 is expressed in human breast cancer cells (Salaun B, Coste I, Rissoan M C, Lebecque S J, Renno T. TLR3 can directly trigger apoptosis in human cancer cells. J Immunol. 2006; 176(8):4894-4901.), hepatocarcinoma and gastric carcinoma cells express TLR2 and TLR4 (Huang B, et al. Listeria monocytogenes promotes tumor growth via tumor cell toll-like receptor 2 signaling. Cancer Res. 2007; 67(9):4346-4352), and TLR9 (Droemann D, et al. Human lung cancer cells express functionally active Toll-like receptor 9. Respir Res. 2005; 6:1.) and TLR4 (He W, Liu Q, Wang L, Chen W, Li N, Cao X. TLR4 signaling promotes immune escape of human lung cancer cells by inducing immunosuppressive cytokines and apoptosis resistance. Mol Immunol. 2007; 44(11):2850-2859.) are expressed by human lung cancer cells. TLR7 and TLR8 are found in tumor cells of human lung cancer (Cherfils-Vicini J, Platonova S, Gillard M, Laurans L, Validire P, Caliandro R, Magdeleinat P, Mami-Chouaib F, Dieu-Nosjean M C, Fridman W H, Damotte D, Sautes-Fridman C, Cremer I. J. Clin Invest. 2010; 120(4):1285-1297).

TLRs are a family of proteins that sense a microbial product and/or initiates an adaptive immune response. TLRs activate a dendritic cell (DC). TLRs are conserved membrane spanning molecules containing an ectodomain of leucine-rich repeats, a transmembrane domain and an intracellular TIR (Toll/interleukin receptor) domain. TLRs recognize distinct structures in microbes, often referred to as “PAMPs” (pathogen associated molecular patterns). Ligand binding to TLRs invokes a cascade of intra-cellular signaling pathways that induce the production of factors involved in inflammation and immunity.

In some embodiments, the immunotherapeutic is a TLR7 and/or TLR8 agonist. TLR7 and TLR8 are phylogenetically and structurally related. TLR7 is selectively expressed by human pDCs and B cells. TLR8 is predominantly expressed mDCs, monocytes, macrophages and myeloid suppressor cells. TLR7-specific agonists activate plasmacytoid DCs (pDCs) to produce large amounts of type 1 IFNs and expressing high levels of costimulatory molecules that promote activation of T cells, NK cells, B cells and mDCs. TLR8-specific agonists activate myeloid DCs, monocytes, macrophages or myeloid-derived suppressor cells to produce large amounts of type 1 IFN, IL-12 and IL-23, and express high levels of MHC class I, MHC class II and costimulatory molecules that promote the activation of antigen specific CD4 and CD8+ T cells.

In some embodiments, the immunotherapeutic is a TLR7 and/or TLR8 agonist that is represented by the structure of Formula (I):

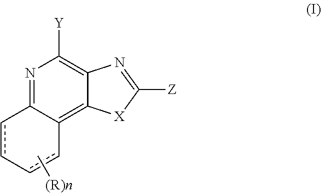

wherein dashed line represents bond or absence of bond;
X is S or —NR₁, R₁ is —W₀—W₁—W₂—W₃—W₄,
W₀ is a bond, alkyl alkenyl, alkynyl, alkoxy, or -alkyl-S-alkyl-,
W₁ is a bond, —O—, or —NR₂—, wherein R₂ is hydrogen, alkyl or alkenyl,
W₂ is a bond, —O—, —C(O)—, —C(S)—, or —S(O)₂—,
W₃ is a bond, —NR₃—, wherein R₃ is hydrogen, alkyl or alkenyl,
W₄ is hydrogen, alkyl, alkenyl, alkynyl, alkoxy, cycloalkyl, aryl, aryloxy, heteroaryl, or heterocyclyl, each of which is optionally substituted by one or more substituents selected from the group consisting of hydroxyl, alkoxy, alkyl, alkenyl, alkynyl, cycloalkyl, aryl, heteroaryl, heterocyclyl, —NH₂, nitro, -alkyl-hydroxyl, -alkyl-aryl, -alkyl-heteroaryl, -alkyl-heterocyclyl, —O—R₄, —O-alkyl-R₄, -alkyl-O—R₄, —C(O)—R₄, -alkyl-C(O)—R₄, -alkyl-C(O)—O—R₄, —C(O)—O—R₄, —S—R₄, —S(O)₂—R₄, —NH—S(O)₂—R₄, -alkyl-S—R₄, -alkyl-S(O)₂—R₄, —NHR₄, —NR₄R₄, —NH-alkyl-R₄, halogen, —CN, —NO₂, and —SH, wherein R₄ is independently hydrogen, alkyl, alkenyl, -alkyl-hydroxyl, aryl, heteroaryl, heterocyclyl, or haloalkyl;
Z is hydrogen, alkyl, alkenyl, alkynyl, alkoxy, aryl, haloalkyl, heteroaryl, heterocyclyl, each of which can be optionally substituted by one or more substituents selected from the group consisting of hydroxyl, alkoxy, alkyl, alkenyl, alkynyl, aryl, heteroaryl, heterocyclyl, halogen, cyano, nitro, —N(R₅)₂, -alkoxy-alkyl, -alkoxy-alkenyl, —C(O)-alkyl, —C(O)—O-alkyl, —O—C(O)-alkyl, —C(O)—N(R₅)₂, aryl, heteroaryl, —CO-aryl, and —CO-heteroaryl, wherein each R₅ is independently hydrogen, alkyl, haloalkyl, -alkyl-aryl, or -alkyl-heteroaryl; R is hydrogen, alkyl, alkoxy, haloalkyl, halogen, aryl, heteroaryl, heterocyclyl, each of which is optionally substituted by one or more substituents selected from the group consisting of hydroxyl, alkoxy, alkyl, alkenyl, alkynyl, cycloalkyl, aryl, heteroaryl, heterocyclyl, —NH₂, nitro, -alkyl-hydroxyl, -alkyl-aryl, -alkyl-heteroaryl, -alkyl-heterocyclyl, —O—R₄, —O-alkyl-R₄, -alkyl-O—R₄, —C(O)—R₄, —C(O)—NH—R₄, —C(O)—NR₄R₄, -alkyl-C(O)—R₄, -alkyl-C(O)—O—R₄, —C(O)—O—R₄, —O—C(O)—R₄, —S—R₄, —C(O)—S—R₄, —S—C(O)—R₄, —S(O)₂—R₄, —NH—S(O)₂—R₄, -alkyl-S—R₄, -alkyl-S(O)₂—R₄, —NHR₄, —NR₄R₄, —NH-alkyl-R₄, halogen, —CN, and —SH, wherein R₄ is independently hydrogen, alkyl, alkenyl, alkoxy, -alkyl-hydroxyl, aryl, heteroaryl, heterocyclyl, or haloalkyl;
n is 0, 1, 2, 3, or 4;
Y is —NR₆R₇, —CR₆R₇R₈, or -alkyl-NH₂, each of which can be optionally substituted by one or more substituents selected from the group consisting of hydroxyl, alkoxy, alkyl, alkenyl, alkynyl, —NH₂, halogen, —N(R₅)₂, -alkoxy-alkyl, -alkoxy-alkenyl, —C(O)-alkyl, —C(O)—O-alkyl, —C(O)—N(R₅)₂, aryl, heteroaryl, —CO-aryl, and —CO-heteroaryl,
wherein R₆, R₇ and R₈ are independently hydrogen, alkyl, alkenyl, alkoxy, alkylamino, dialkylamino, alkylthio, arylthio, -alkyl-hydroxyl, -alkyl-C(O)—O—R₉, -alkyl-C(O)—R₉, or -alkyl-O—C(O)—R₉, wherein each R₅ is independently hydrogen, alkyl, haloalkyl, -alkyl-aryl, or -alkyl-heteroaryl, wherein R₉ is hydrogen, alkyl, alkenyl, halogen, or haloalkyl;
X and Z taken together may optionally form a (5-9)-membered ring;
or a pharmaceutically acceptable salt or solvate thereof.

In some embodiments, X of Formula (I) is S.

In some embodiments, X of Formula (I) is —NR₁, R₁ is alkyl, -alkyl-W₄, -alkyl-O—W₄, -alkyl-NH—C(O)—W₄, -alkoxy-NH—C(O)—W₄, -alkyl-NH—C(O)—NH—W₄, -alkoxy-NH—C(O)—NH—W₄, -alkyl-S(O)₂—W₄, or -alkyl-NH—C(S)—W₄, wherein W₄ is defined above.

In some embodiments, Z of Formula (I) is hydrogen, alkyl, alkoxy, aryl, heteroaryl, haloalkyl, each of which is optionally substituted by one to three substituents selected from the group consisting of hydroxyl, alkyl, aryl, heteroaryl, heterocyclyl, cyano, -alkoxy-alkyl, nitro, and —N(R₅)₂, wherein each R₅ is independently hydrogen, alkyl, haloalkyl, -alkyl-aryl, or -alkyl-heteroaryl.

In some embodiments, Y of Formula (I) is —NH₂, -alkyl-NH₂, each of which is optionally substituted by one to three substituents selected from the group consisting of alkyl, alkoxy, alkenyl, and alkynyl.

In some embodiments, n of Formula (I) is 1 or 2.

In some embodiments, R of Formula (I) is aryl or heteroaryl each of which is optionally substituted by one to three substituents selected from the group consisting of hydroxyl, alkoxy, -alkyl-hydroxyl, —O—R₄, —O-alkyl-R₄, -alkyl-O—R₄, —C(O)—R₄, —C(O)—NH—R₄, —C(O)—NR₄R₄, -alkyl-C(O)—R₄, -alkyl-C(O)—O—R₄, —C(O)—O—R₄, —O—C(O)—R₄, —S—R₄, —C(O)—S—R₄, —S—C(O)—R₄, —S(O)₂—R₄,

—NH—S(O)₂—R₄, -alkyl-S—R₄, -alkyl-S(O)₂—R₄, —NHR₄, —NR₄R₄, —NH-alkyl-R₄, halogen, —CN, and —SH, wherein R₄ is independently hydrogen, alkyl, alkenyl, alkoxy, -alkyl-hydroxyl, aryl, heteroaryl, heterocyclyl, or haloalkyl.

In some embodiments, the immunotherapeutic is a TLR7 and/or TLR8 agonist that is selected from Table 2. The compounds in Table 2 are described and characterized in more details in U.S. Pat. Nos. 4,689,338, 5,389,640, 5,226,575, 6,110,929, 6,194,425, 5,352,784, 6,331,539, 5,482,936, 6,451,810, WO2002/46192, WO2002/46193, WO2002/46194, US2004/0014779 and US2004/0162309.

TABLE 2
Representative TLR7 and/or TLR8 Agonists

| Name | Structure |
|---|---|
| 2-propylthiazolo[4,5- c]quinolin-4-amine (CL075) | 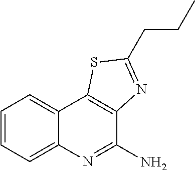 [SMILES: CCCc1nc2c(N)nc3ccccc3c2s1] |
| 1-(2-methylpropyl)-1H- imidazo[4,5-c]quinolin-4- amine (Imiquimod) | 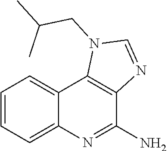 [SMILES: CC(C)Cn1cnc2c(N)nc3ccccc3c21] |
| 4-amino-2- (ethoxymethyl)-a,a-di- methyl-1H-imidazo[4,5- c]quinoline-1-ethanol (Resiquimod) | 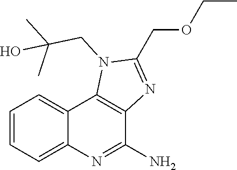 [SMILES: CCOCc1nc2c(N)nc3ccccc3c2n1CC(C)(C)O] |
| 1-(4-amino-2- ethylaminomethylimidazo- [4,5-c]quinolin-1-yl)-2- methylpropan-2-ol (Gardiquimod) | 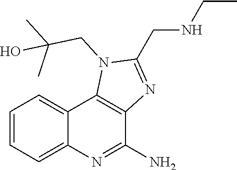 [SMILES: CCNCc1nc2c(N)nc3ccccc3c2n1CC(C)(C)O] |
| N-[4-(4-amino-2-ethyl- 1H-imidazo[4,5- c]quinolin-1- yl)butyl-]methanesulfonamide (CM001) | 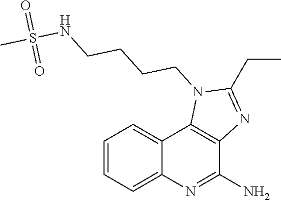 [SMILES: CCc1nc2c(N)nc3ccccc3c2n1CCCCNS(C)(=O)=O] |
| 7-allyl-7,8-dihydro-8- oxo-guanosine (Loxoribine) | 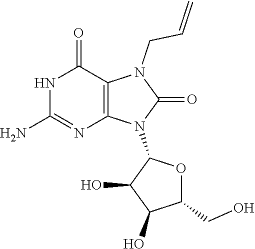 [SMILES: C=CCn1c(=O)n([C@@H]2O[C@H](CO)[C@@H](O)[C@H]2O)c2nc(N)[nH]c(=O)c21] |
| 4-amino-2-ethoxymethyl- aa-dimethyl-6,7,8,9- tetrahydro-1h- imidazo[4,5-c]quinoline- 1-ethanol ol | 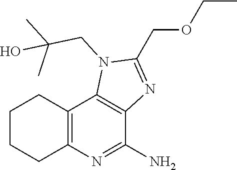 [SMILES: CCOCc1nc2c(N)nc3c(c2n1CC(C)(C)O)CCCC3] |
| 4-amino-aa-dimethyl-2- methoxyethyl-1h- imidazo[4,5- c]quinoline-1-ethanol | 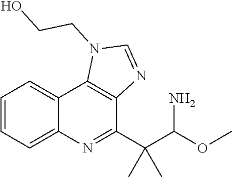 [SMILES: COC(N)C(C)(C)c1nc2ccccc2c2c1ncn2CCO] |
| 1-(2-(3- (benzyloxy)propoxy)ethyl)- 2-(ethoxymethyl)-1H- imidazo[4,5-c]quinolin-4- amine | 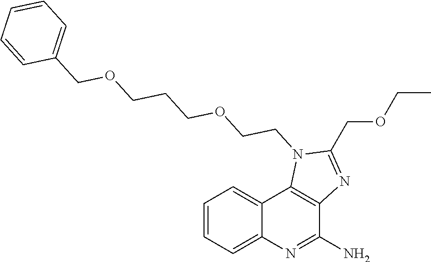 [SMILES: CCOCc1nc2c(N)nc3ccccc3c2n1CCOCCCOCc1ccccc1] |
| N-[4-(4-amino-2-butyl- 1H-imidazo[4,5- c][1,5]naphthyridin-1- yl)butyl]-n′-butylurea | 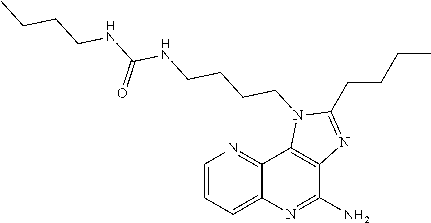 [SMILES: CCCCNC(=O)NCCCCn1c(CCCC)nc2c(N)nc3cccnc3c21] |
| N1-[2-(4-amino-2-butyl- 1H-imidazo[4,5-c][1,5] naphthyridin-1- yl)ethyl]-2-amino-4- methylpentanamide | 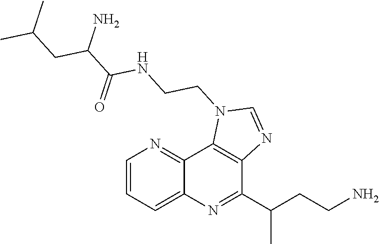 [SMILES: CC(C)CC(N)C(=O)NCCn1cnc2c(C(C)CCN)nc3cccnc3c21] |
| N-(2-{2-[4-amino-2-(2- methoxyethyl)-1H- imidazo[4,5-c]quinolin- 1-yl]ethoxy}ethyl)-n′- phenylurea | 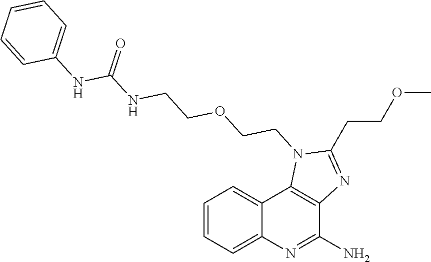 [SMILES: COCCc1nc2c(N)nc3ccccc3c2n1CCOCCNC(=O)Nc1ccccc1] |
| 1-(2-amino-2- methylpropyl)-2- (ethoxymethyl)-1H- imidazo[4,5-c]quinolin- 4-amine | 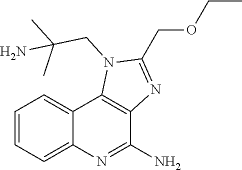 [SMILES: CCOCc1nc2c(N)nc3ccccc3c2n1CC(C)(C)N] |
| 1-{4-[(3,5- dichlorophenyl)sulfonyl] butyl}-2-ethyl- 1H-imidazo[4,5- c]quinolin-4-amine | 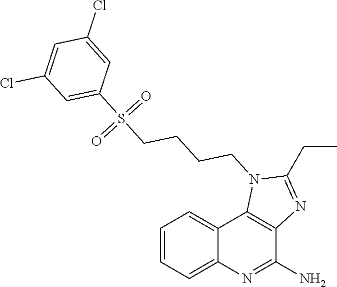 [SMILES: CCc1nc2c(N)nc3ccccc3c2n1CCCCS(=O)(=O)c1cc(Cl)cc(Cl)c1] |
| N-(2-{2-[4-amino-2- (ethoxymethyl)-1H- imidazo[4,5- c]quinolin-1- yl]ethoxy}ethyl)-n′- cyclohexylurea | 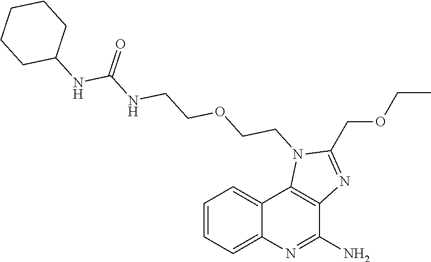 [SMILES: CCOCc1nc2c(N)nc3ccccc3c2n1CCOCCNC(=O)NC1CCCCC1] |
| N-{3-[4-amino-2- (ethoxymethyl)-1H- imidazo[4,5- c]quinolin-1-yl]propyl}- n′-(3- cyanophenyl)thiourea | 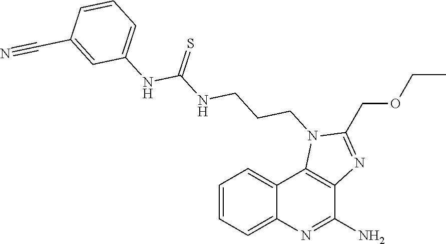 [SMILES: CCOCc1nc2c(N)nc3ccccc3c2n1CCCNC(=S)Nc1cccc(C#N)c1] |
| N-[3-(4-amino-2-butyl- 1H-imidazo[4,5- c]quinolin-1- yl)-2,2- dimethylpropyl]benzamide | 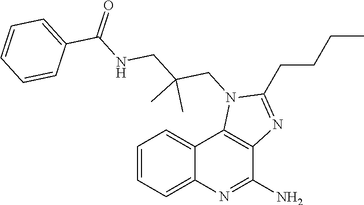 [SMILES: CCCCc1nc2c(N)nc3ccccc3c2n1CC(C)(C)CNC(=O)c1ccccc1] |
| 2-butyl-1-[3- (methylsulfonyl)propyl]- 1H- imidazo[4,5-c]quinolin- 4-amine | 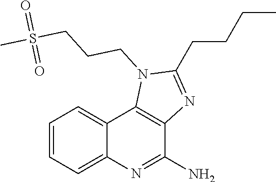 [SMILES: CCCCc1nc2c(N)nc3ccccc3c2n1CCCS(C)(=O)=O] |
| N-{2-[4-amino-2- (ethoxymethyl)-1H- imidazo[4,5- c]quinolin-1-yl]-1,1- dimethylethyl}-2- ethoxyacetamide | 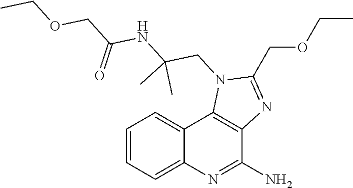 [SMILES: CCOCC(=O)NC(C)(C)Cn1c(COCC)nc2c(N)nc3ccccc3c21] |
| 1-[4-amino-2- ethoxymethyl-7-(pyridin- 4-yl)-1H- imidazo[4,5-c]quinolin- 1-yl]-2-methylpropan-2- ol | 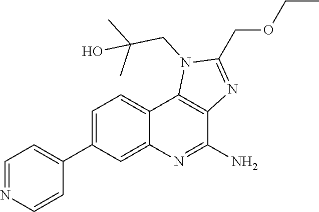 [SMILES: CCOCc1nc2c(N)nc3cc(-c4ccncc4)ccc3c2n1CC(C)(C)O] |
| 1-[4-amino-2- (ethoxymethyl)-7- (pyridin-3-yl)-1H- imidazo[4,5-c]quinolin- 1-yl]-2-methylpropan-2- ol | 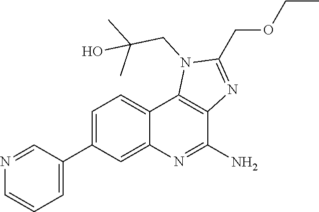 [SMILES: CCOCc1nc2c(N)nc3cc(-c4cccnc4)ccc3c2n1CC(C)(C)O] |
| N-{3-[4-amino-1-(2- hydroxy-2- methylpropyl)-2- (methoxyethyl)-1H- imidazo[4,5-c]quinolin-7- yl]phenyl} methanesulfonamide | 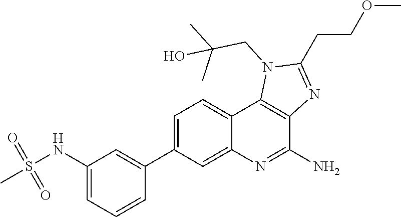 |
| 1-[4-amino-7-(5- hydroxymethylpyridin-3- yl)-2-(2- methoxyethyl)-1H- imidazo[4,5-c]quinolin-1- yl]-2- methylpropan-2-ol | 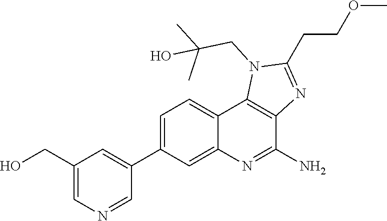 [SMILES: COCCc1nc2c(N)nc3cc(-c4cncc(CO)c4)ccc3c2n1CC(C)(C)O] |
| 3-[4-amino-2- (ethoxymethyl)-7- (pyridin-3-yl)-1H- imidazo[4,5-c]quinolin- 1-yl]propane-1,2-diol | 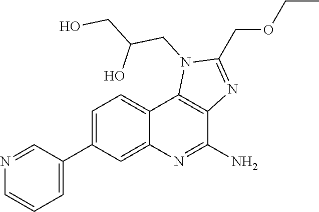 [SMILES: CCOCc1nc2c(N)nc3cc(-c4cccnc4)ccc3c2n1CC(O)CO] |
| 1-[2-(4-amino-2- ethoxymethyl-1H- imidazo[4,5- c]quinolin-1-yl)-1,1- dimethylethyl]-3- propylurea | 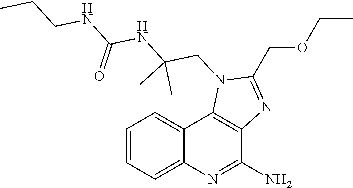 [SMILES: CCCNC(=O)NC(C)(C)Cn1c(COCC)nc2c(N)nc3ccccc3c21] |
| 1-[2-(4-amino-2- ethoxymethyl-1H- imidazo[4,5- c]quinolin-1-yl)-1,1- dimethylethyl]-3- cyclopentylurea | 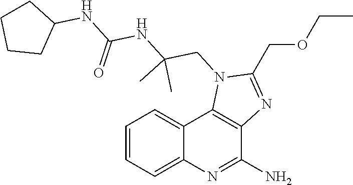 [SMILES: CCOCc1nc2c(N)nc3ccccc3c2n1CC(C)(C)NC(=O)NC1CCCC1] |
| 1-[(2,2-dimethyl-1,3- dioxolan-4-yl)methyl]-2- (ethoxymethyl)-7-(4- hydroxymethylphenyl)- 1H- imidazo[4,5-c]quinolin- 4-amine | 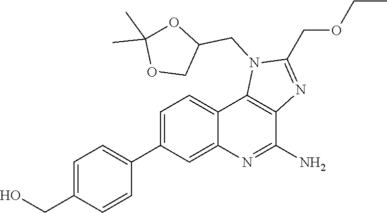 [SMILES: CCOCc1nc2c(N)nc3cc(-c4ccc(CO)cc4)ccc3c2n1CC1COC(C)(C)O1] |
| 4-[4-amino-2- ethoxymethyl-1-(2- hydroxy-2- methylpropyl)-1H- imidazo[4,5-c]quinolin-7- yl]-N- methoxy-N- methylbenzamide | 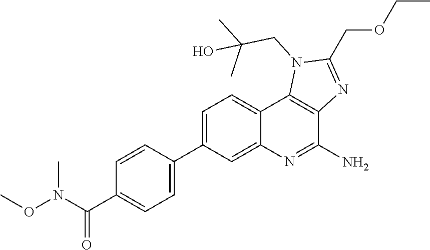 [SMILES: CCOCc1nc2c(N)nc3cc(-c4ccc(C(=O)N(C)OC)cc4)ccc3c2n1CC(C)(C)O] |
| 2-ethoxymethyl-N1- isopropyl-6,7,8,9- tetrahydro-1H- imidazo[4,5- c]quinoline-1,4-diamine | 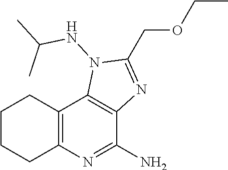 [SMILES: CCOCc1nc2c(N)nc3c(c2n1NC(C)C)CCCC3] |
| 1-[4-amino-2-ethyl-7- (pyridin-4-yl)-1H- imidazo[4,5- c]quinolin-1-yl]-2- methylpropan-2-ol | 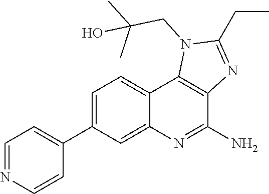 [SMILES: CCc1nc2c(N)nc3cc(-c4ccncc4)ccc3c2n1CC(C)(C)O] |
| N-[4-(4-amino-2-ethyl- 1H-imidazo[4,5- c]quinolin-1- yl)butyl]methanesulfonamide | 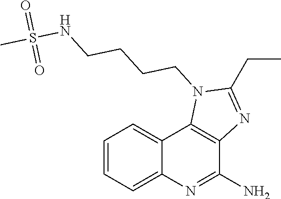 [SMILES: CCc1nc2c(N)nc3ccccc3c2n1CCCCNS(C)(=O)=O] |
| N-[4-(4-amino-2-butyl- 1H-imidazo[4,5- c][1,5]naphthyridin-1- yl)butyl]-n′- cyclohexylurea | 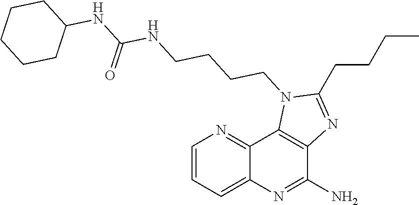 [SMILES: CCCCc1nc2c(N)nc3cccnc3c2n1CCCCNC(=O)NC1CCCCC1] |
| 3M-34240 | 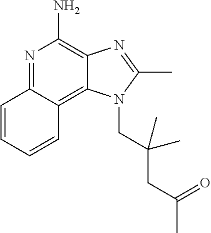 [SMILES: CC(=O)CC(C)(C)Cn1c(C)nc2c(N)nc3ccccc3c21] |
| 3M-052 | 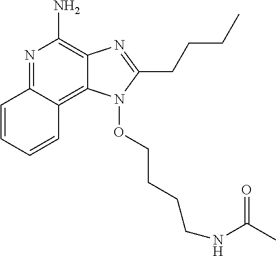 [SMILES: CCCCc1nc2c(N)nc3ccccc3c2n1OCCCCNC(C)=O] |
| 3M-854A | 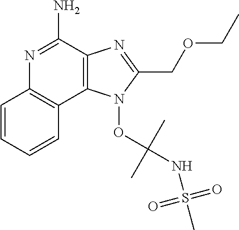 [SMILES: CCOCc1nc2c(N)nc3ccccc3c2n1OC(C)(C)NS(C)(=O)=O] |

Preferably in some embodiments, the immunotherapeutic is Resiquimod or Imiquimod.

In some embodiments, the immunotherapeutic is a TLR modulator (e.g., TLR7 and/or TLR8 agonist) that is represented by structure of Formula (II):

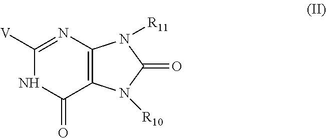

wherein V is —NR₆R₇, wherein each of R₆ and R₇ is independently hydrogen, alkyl, alkenyl, alkoxy, alkylamino, dialkylamino, alkylthio, arylthio, -alkyl-hydroxyl, -alkyl-C(O)—O—R₉, -alkyl-C(O)—R₉, or -alkyl-O—C(O)—R₉, wherein R₉ is hydrogen, alkyl, alkenyl, hydrogen, or haloalkyl;
R₁₀ and R₁₁ are independently hydrogen, alkyl, alkenyl, aryl, haloalkyl, heteroaryl, heterocyclyl, or cycloalkyl, each of which is optionally substituted by one or more substituents selected from the group consisting of hydroxyl, alkoxy, alkyl, alkenyl, alkynyl, halogen, —N(R₅)₂, -alkoxy-alkyl, -alkoxy-alkenyl, —C(O)-alkyl, —C(O)—O-alkyl, —C(O)—N(R₅)₂, aryl, heteroaryl, —CO-aryl, and —CO-heteroaryl, wherein each R₅ is independently hydrogen, alkyl, haloalkyl, -alkyl-aryl, or -alkyl-heteroaryl, or a pharmaceutically acceptable salt or solvate thereof.

In some embodiments, the immunotherapeutic is a TLR modulator (e.g., TLR7 and/or TLR8 agonist) that is represented by structure of Formula (III):

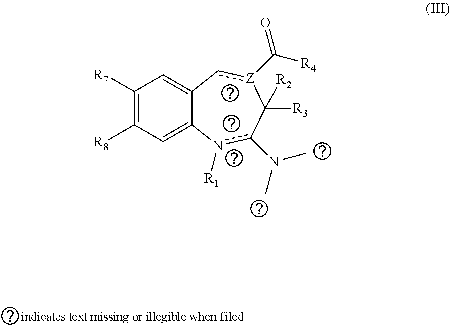

wherein [symbol] is a double bond or a single bond; R₂ and R₃ are independently selected from H and lower alkyl, or R₂ and R₃ are connected to form a saturated carbocycle having from 3 to 7 ring members; one of R₇ and R₈ is

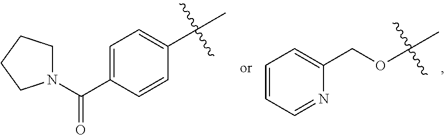 [SMILES: *c1ccc(C(=O)N2CCCC2)cc1 ; *OCc1ccccn1]

and the other is hydrogen; R₄ is —NR_{c}R_{d} or —OR₁₀; R_{c} and R_{d} are lower alkyl, where the alkyl is optionally substituted with one or more —OH; R₁₀ is alkyl, where the alkyl is optionally substituted with one or more —OH; Z is C and [symbol] is a double bond, or Z is N and [symbol] is a single bond; Rₐ and R_{b} are independently selected from H, alkyl, alkenyl, alkynyl, and R_{c}, wherein the alkyl is optionally substituted with one or more —OR₁₀, or R_{c}, R_{c} is selected from —NH₂, —NH(alkyl), and —N(alkyl)₂; R₁ is absent when [symbol] is a double bond, or when [symbol] is a single bond, N₁—R₁ and one of Rₐ or R_{b} are connected to form a saturated, partially unsaturated, or unsaturated heterocycle having 5-7 ring members and the other of Rₐ or R_{b} may be hydrogen or absent as necessary to accommodate ring unsaturation; and at least one of the following A-D applies: A) R₇ is not hydrogen B) R₈ is not hydrogen and at least one of Rₐ and R_{b} is not hydrogen; C) Z is N; or D) N₁—R₁ and one of Rₐ or R_{b} are connected to form a saturated, partially unsaturated, or unsaturated heterocycle having 5-7 ring members. US 20140088085A1, the disclosure of which is incorporated by references in its entirety.

In some embodiments, R₇ of the compound of Formula (III) is

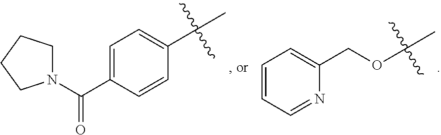 [SMILES: *c1ccc(C(=O)N2CCCC2)cc1 ; *OCc1ccccn1]

Additionally, at least one of Rₐ and R_{b} is not hydrogen in the compound of Formula (III), or, for example, one of Rₐ and R_{b} is alkyl and the other of Rₐ and R_{b} is hydrogen. Further, the alkyl of Formula (III) is substituted with R_{c}. In a different embodiment, both Rₐ and R_{b} are alkyl or, one of Rₐ and R_{b} is R_{c} and the other Rₐ and R_{b} is hydrogen. For example, R₈ of formula (III) is not hydrogen.

In some alternative embodiments, N, and one of Rₐ or R_{b} of Formula (III) are connected to form a saturated, partially unsaturated, or unsaturated heterocycle having 5-7 ring members and the other of Rₐ or R_{b} is hydrogen, or absent as necessary to accommodate ring unsaturation, where the ring is a 5 membered ring, or, for example, the ring is:

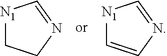

In some embodiments, at least one of R₂ and R₃ in the compound of Formula (III) is not hydrogen, or, for example, R₂ and R₃ are connected to form a saturated carbocycle, where the saturated carbocycle is cyclopropyl. Alternatively, Z is N in the compound of Formula (III).

In some embodiments, the TLR agonist or modulator has the structure of Formula (IV):

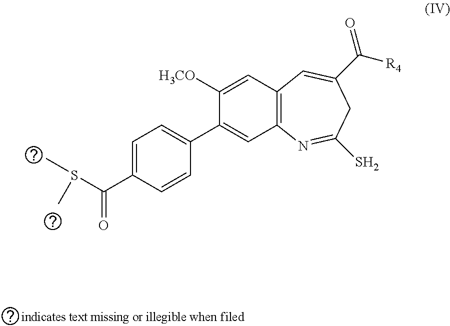

wherein R₄ is selected from —NR_{c}R_{d} and —OR₁₀; R_{c} and R_{d} are lower alkyl, where the alkyl is optionally substituted with one or more —OH; R₁₀ is alkyl, where the alkyl is optionally substituted with one or more —OH; R_{f} and R_{g} are lower alkyl or R_{f} and R_{g} together with the nitrogen atom to which they are attached form a saturated heterocyclic ring having 4-6 ring members. For example, R_{f} and R_{g} in the compound of Formula (IV), together with the nitrogen atom to which they are attached form a saturated heterocyclic ring, where the heterocyclic ring is pyrrolidine.

In some alternative embodiments, R₄ of either Formula (III) or Formula (IV) is —OR₁₀, where R₁₀ is alkyl or is ethyl. In another embodiment, R₄ of either Formula (III) or Formula (IV) is —NR_{c}R_{d}, where both are alkyl or both are propyl. Moreover, in certain embodiments, at least one of R_{c} or R_{d} is alkyl substituted with one —OH and at least one of R_{c} and R_{d} is

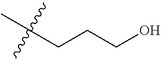 [SMILES: *CCCO]

and the remaining R_{c} or R_{d} is propyl.

In some alternative embodiments, the TLR is a a compound selected from

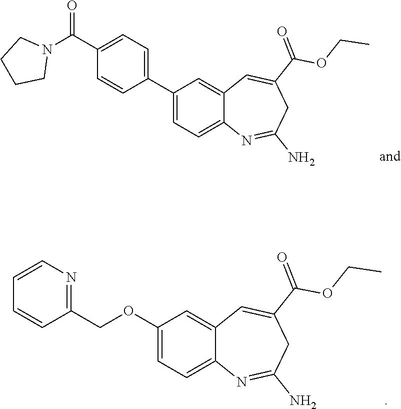 [SMILES: CCOC(=O)C1=Cc2cc(-c3ccc(C(=O)N4CCCC4)cc3)ccc2N=C(N)C1 ; CCOC(=O)C1=Cc2cc(OCc3ccccn3)ccc2N=C(N)C1]

Alternatively, the compound is selected from

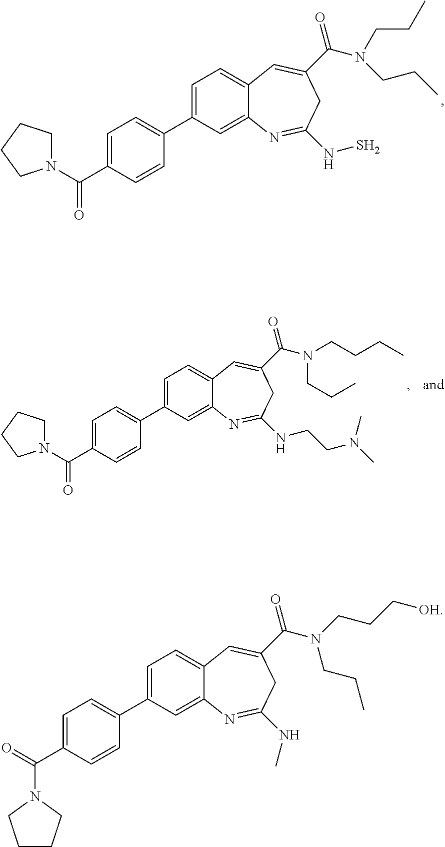 [SMILES: CCCN(CCC)C(=O)C1=Cc2ccc(-c3ccc(C(=O)N4CCCC4)cc3)cc2N=C(NS)C1 ; CCCCN(CCC)C(=O)C1=Cc2ccc(-c3ccc(C(=O)N4CCCC4)cc3)cc2N=C(NCCN(C)C)C1 ; CCCN(CCCO)C(=O)C1=Cc2ccc(-c3ccc(C(=O)N4CCCC4)cc3)cc2N=C(NC)C1]

In some alternative embodiments, the TLR agonist compound is either

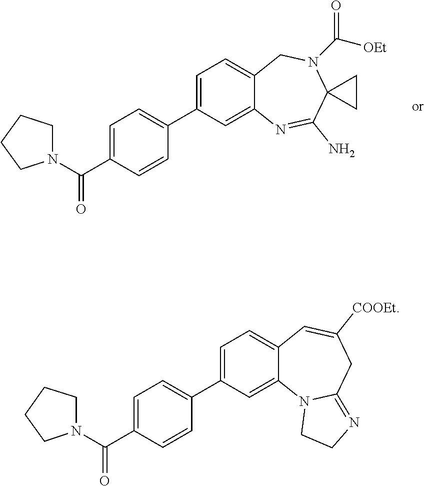 [SMILES: CCOC(=O)N1Cc2ccc(-c3ccc(C(=O)N4CCCC4)cc3)cc2N=C(N)C12CC2 ; CCOC(=O)C1=Cc2ccc(-c3ccc(C(=O)N4CCCC4)cc3)cc2N2CCN=C2C1]

In some alternative embodiments, the TLR agonist is a compound selected from

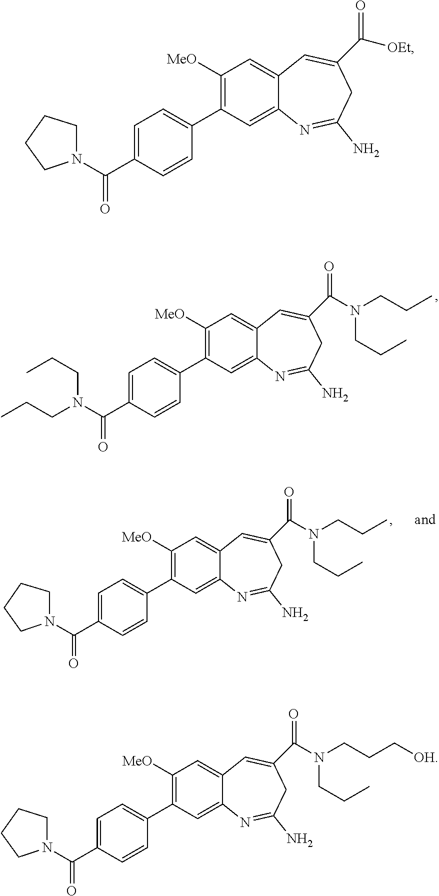 [SMILES: CCOC(=O)C1=Cc2cc(OC)c(-c3ccc(C(=O)N4CCCC4)cc3)cc2N=C(N)C1 ; CCCN(CCC)C(=O)C1=Cc2cc(OC)c(-c3ccc(C(=O)N(CCC)CCC)cc3)cc2N=C(N)C1 ; CCCN(CCC)C(=O)C1=Cc2cc(OC)c(-c3ccc(C(=O)N4CCCC4)cc3)cc2N=C(N)C1 ; CCCN(CCCO)C(=O)C1=Cc2cc(OC)c(-c3ccc(C(=O)N4CCCC4)cc3)cc2N=C(N)C1]

In some alternative embodiments, the TLR agonist is

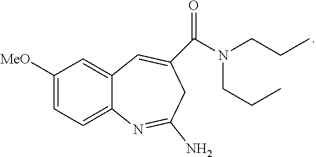 [SMILES: CCCN(CCC)C(=O)C1=Cc2cc(OC)ccc2N=C(N)C1]

In some alternative embodiments, the TLR agonist is a compound selected from:

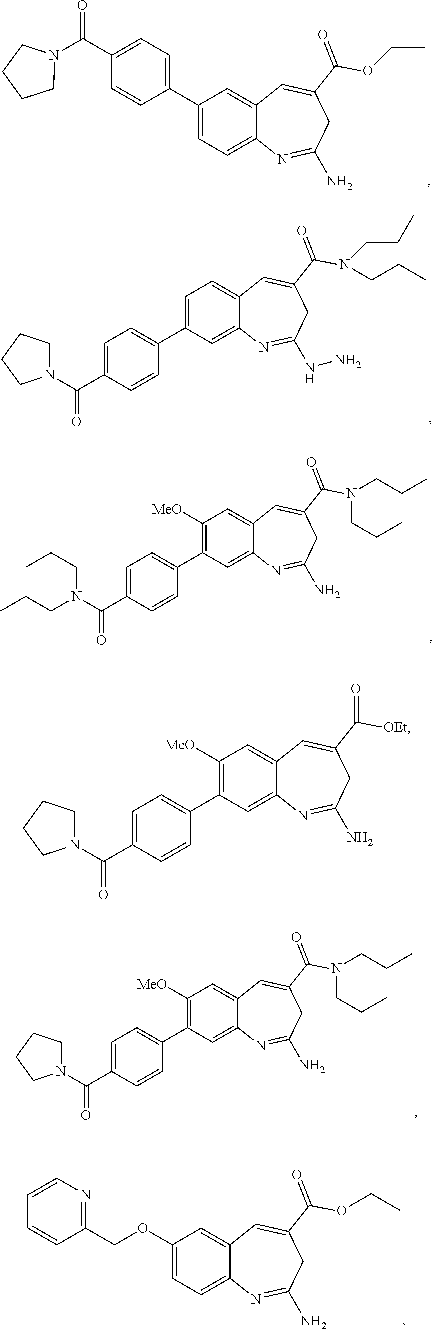

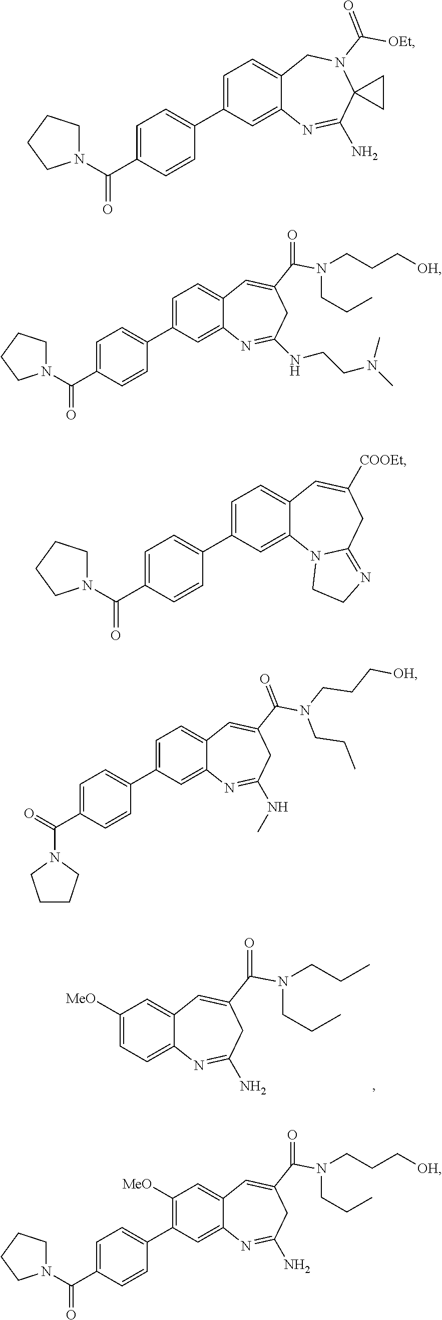 [SMILES: CCOC(=O)N1Cc2ccc(-c3ccc(C(=O)N4CCCC4)cc3)cc2N=C(N)C12CC2 ; CCCN(CCCO)C(=O)C1=Cc2ccc(-c3ccc(C(=O)N4CCCC4)cc3)cc2N=C(NCCN(C)C)C1 ; CCOC(=O)C1=Cc2ccc(-c3ccc(C(=O)N4CCCC4)cc3)cc2N2CCN=C2C1 ; CCCN(CCCO)C(=O)C1=Cc2ccc(-c3ccc(C(=O)N4CCCC4)cc3)cc2N=C(NC)C1 ; CCCN(CCC)C(=O)C1=Cc2cc(OC)ccc2N=C(N)C1 ; CCCN(CCCO)C(=O)C1=Cc2cc(OC)c(-c3ccc(C(=O)N4CCCC4)cc3)cc2N=C(N)C1]

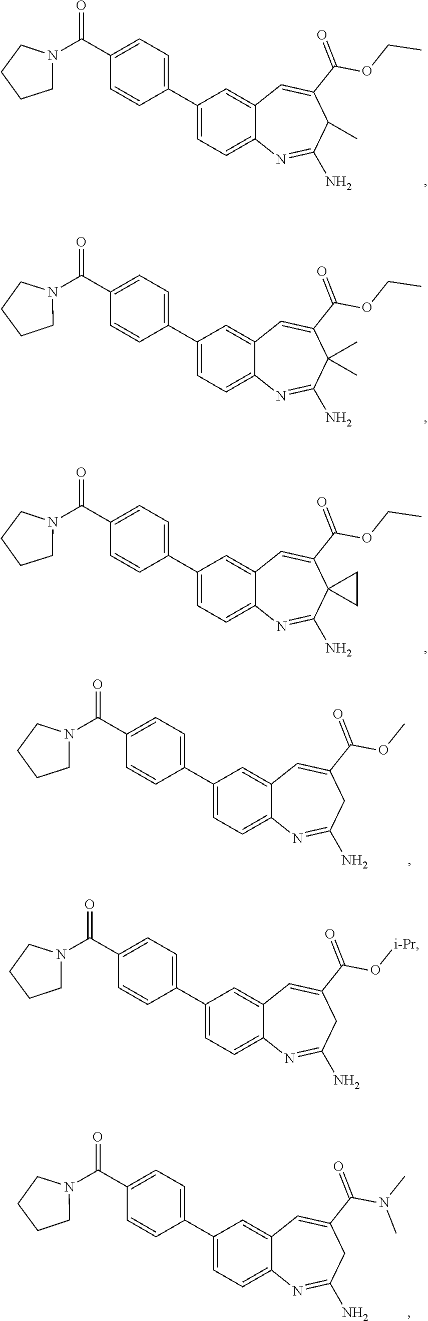 [SMILES: CCOC(=O)C1=Cc2cc(-c3ccc(C(=O)N4CCCC4)cc3)ccc2N=C(N)C1C ; CCOC(=O)C1=Cc2cc(-c3ccc(C(=O)N4CCCC4)cc3)ccc2N=C(N)C1(C)C ; CCOC(=O)C1=Cc2cc(-c3ccc(C(=O)N4CCCC4)cc3)ccc2N=C(N)C12CC2 ; COC(=O)C1=Cc2cc(-c3ccc(C(=O)N4CCCC4)cc3)ccc2N=C(N)C1 ; CC(C)OC(=O)C1=Cc2cc(-c3ccc(C(=O)N4CCCC4)cc3)ccc2N=C(N)C1 ; CN(C)C(=O)C1=Cc2cc(-c3ccc(C(=O)N4CCCC4)cc3)ccc2N=C(N)C1]

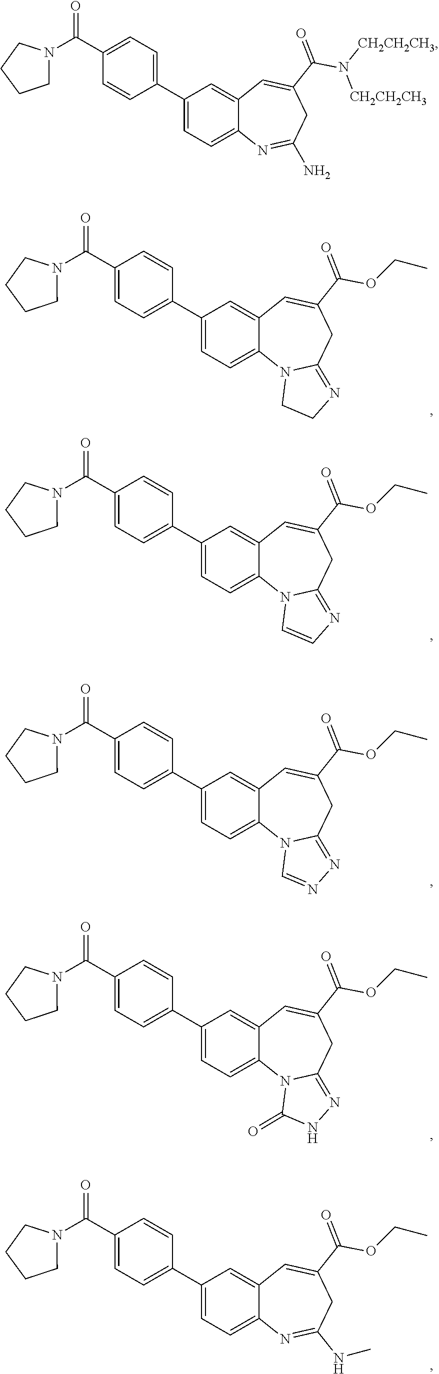

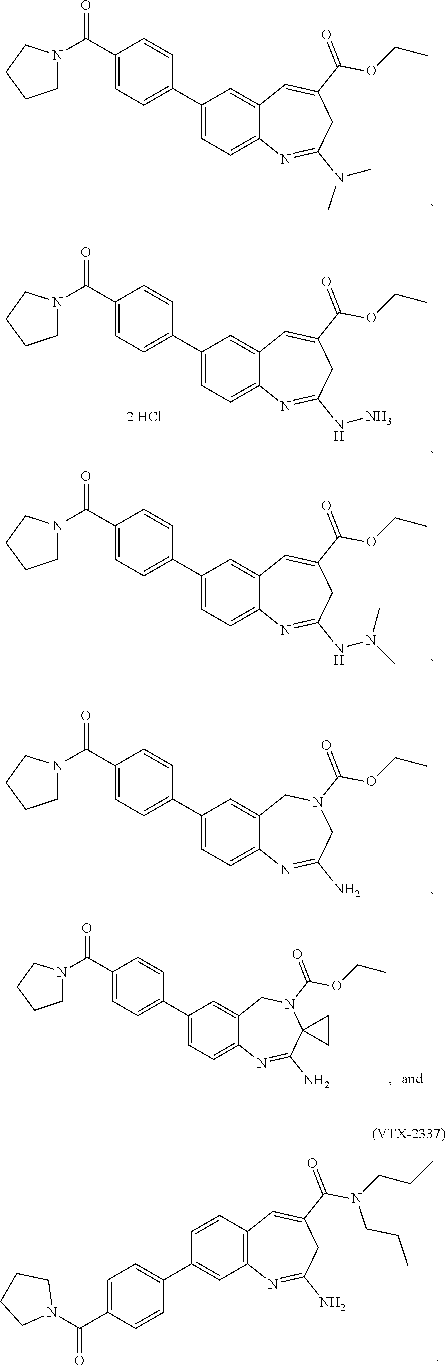 [SMILES: CCOC(=O)C1=Cc2cc(-c3ccc(C(=O)N4CCCC4)cc3)ccc2N=C(N(C)C)C1 ; CCOC(=O)C1=Cc2cc(-c3ccc(C(=O)N4CCCC4)cc3)ccc2N=C(NN)C1 ; CCOC(=O)C1=Cc2cc(-c3ccc(C(=O)N4CCCC4)cc3)ccc2N=C(NN(C)C)C1 ; CCOC(=O)N1CC(N)=Nc2ccc(-c3ccc(C(=O)N4CCCC4)cc3)cc2C1 ; CCOC(=O)N1Cc2cc(-c3ccc(C(=O)N4CCCC4)cc3)ccc2N=C(N)C12CC2 ; CCCN(CCC)C(=O)C1=Cc2ccc(-c3ccc(C(=O)N4CCCC4)cc3)cc2N=C(N)C1]

In some embodiments, the immunotherapeutic is a TLR modulator (e.g., TLR7 and/or TLR8 agonist) that is represented by structure of Formula (V):

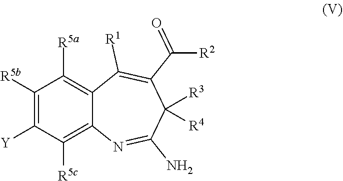

and metabolites, solvates, tautomers, and prodrugs thereof, wherein:

Y is CF₂CF₃, CF₂CF₂R⁶, or an aryl or heteroaryl ring, wherein said aryl and heteroaryl rings are substituted with one or more groups independently selected from alkenyl, alkynyl, Br, CN, OH, NR⁶R⁷, C(═O)R⁸, NR⁶SO₂R⁷, (C₁-C₆ alkyl)amino, R⁶OC(═O)CH═CH₂—, SR⁶ and SO₂R⁶, and wherein the aryl and heteroaryl rings are optionally further substituted with one or more groups independently selected from F, Cl, CF₃, CF₃O—, HCF₂O—, alkyl, heteroalkyl and ArO—;

R¹, R³ and R⁴ are independently selected from H, alkyl, alkenyl, alkynyl, heteroalkyl, cycloalkyl, cycloalkenyl, heterocycloalkyl, aryl and heteroaryl, wherein the alkyl, alkenyl, alkynyl, heteroalkyl, cycloalkyl, cycloalkenyl, heterocycloalkyl, aryl and heteroaryl are optionally substituted with one or more groups independently selected from alkyl, alkenyl, alkynyl, F, Cl₅ Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(\hat{ }O)CH═CH₂—, NR⁶SO₂R⁷, SR⁶ and SO₂R⁶,

or R³ and R⁴ together with the atom to which they are attached form a saturated or partially unsaturated carbocyclic ring, wherein the carbocyclic ring is optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(═O)CH═CH₂—, NR⁶SO₂R⁷, SR⁶ and SO₂R⁶;

R² and R⁸ are independently selected from H, OR⁶, NR⁶R⁷, alkyl, alkenyl, alkynyl, heteroalkyl, cycloalkyl, cycloalkenyl, heterocycloalkyl, aryl and heteroaryl, wherein the alkyl, alkenyl, alkynyl, heteroalkyl, cycloalkyl, cycloalkenyl, heterocycloalkyl, aryl and heteroaryl are optionally substituted with one or more groups independently selected from alkyl, alkenyl, alkynyl, F, Cl, Br₅ I, CN, OR⁶, NR⁶R⁷, C(═O)R⁶, C(═)OR⁶, OC(═O)R⁶, C(\hat{ }O)NR⁶R⁷, (C₁-C₆ alkyl)amino, CH₃OCH₂O—, R⁶OC(═O)CH═CH₂—, NR⁶SO₂R⁷, SR⁶ and SO₂R⁶;

R^{5a}, R^{5b}, and R^{5c} are independently H, F, Cl, Br, I, OMe, CH₃, CH₂F₅ CHF₂ or CF₃; and

R⁶ and R⁷ are independently selected from Hs alkyl, alkenyl, alkynyl, heteroalkyl, cycloalkyl, cycloalkenyl, heterocycloalkyl, aryl and heteroaryl, wherein said alkyl, alkenyl, alkynyl, heteroalkyl, cycloalkyl, cycloalkenyl, heterocycloalkyl, aryl and heteroaryl are optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(═O)CH═CH₂—, NR⁶SO₂R⁷, SR⁶ and SO₂R⁶,

or R⁶ and R⁷ together with the atom to which they are attached form a saturated or partially unsaturated heterocyclic ring, wherein said heterocyclic ring is optionally substituted with one or more groups independently selected from alkyl, alkenyl, alkynyl, F, Cl, Br, I, CN, OR⁶, NR⁶R⁷, C(O)R⁶, C(═)OR⁶, OC(═O)R⁶, C(═O)NR⁶R⁷, (C₁-C₆alkyl)amino, CH₃OCH₂O—, R⁶OC(═O)CH═CH₂—, NR⁶SO₂R⁷, SR⁶ and SO₂R⁶. In certain embodiments, R¹, R³ and R⁴ are each hydrogen. In certain embodiments, R^{5a}, R^{5b} and R^{5c} are each hydrogen. WO 2007024612 A2, the disclosure of which is incorporated by reference in its entirety.

In some embodiments of the compound of Formula (V), R² is OR⁶. In some embodiments, R⁶ is alkyl, such as (C₁₋₄)alkyl. In particular embodiments, R⁶ is ethyl.

In some embodiments of the compound of Formula (V), R² is NR⁶R⁷. In some embodiments, R⁶ and R⁷ are independently H, alkyl, such as (C₁₋₆)alkyl, or heteroalkyl, such as (C₁₋₄)alkoxyC₂₋₄)alkyl. In particular embodiments, R⁶ and R⁷ are independently H, ethyl, propyl, or CH₂CH₂OCH₃. In some embodiments of the compound of Formula V, Y is aryl, such as phenyl. In some embodiments, the aryl is substituted with C(═O)R⁸, such as in para-R⁸C(═O)phenyl. In some embodiments, R⁸ is OR⁶, NR⁶R⁷ or heterocycloalkyl. In some embodiments, R⁶ and R⁷ are independently H or alkyl, such as (C₁₋₆)alkyl. In some other embodiments, R⁶ and R⁷ together with the nitrogen atom to which they are attached form a 4-6 membered azacycloalkyl ring, such as pyrrolidinyl. In some embodiments, Y is

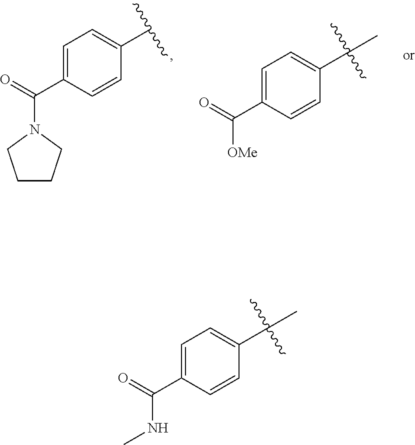 [SMILES: *c1ccc(C(=O)N2CCCC2)cc1 ; *c1ccc(C(=O)OC)cc1 ; *c1ccc(C(=O)NC)cc1]

In some embodiments of the compound of Formula (V), Y is CF₂CF₃.

In some embodiments, the immunotherapeutic is a TLR modulator (e.g., TLR8 agonist) that is represented by structure of formula (VI):

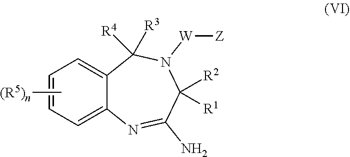

and metabolites, solvates, tautomers, and pharmaceutically acceptable prodrugs and salts thereof, wherein:

Z is H, alkyl, alkenyl, alkynyl, heteroalkyl, cycloalkyl, heterocycloalkyl, aryl, heteroaryl, OR⁶ or NR⁶R⁷, wherein said alkyl, alkenyl, alkynyl; heteroalkyl, cycloalkyl, heterocycloalkyl, aryl and heteroaryl are optionally substituted with one or more groups independently selected from alkyl, alkenyl, alkynyl, F, Cl₃ Br, I, CN, OR⁶, NR⁶R⁷, C(═O)R⁶, C(═O)OR⁶, OC(═O)R⁶, C(═O)NR⁶R⁷, CCi-C₆alkyl)amino, CH₃OCH₂O—, R⁶OCC═O)CH═CH₂—, NR⁶SO₂R⁷, SR⁶ and SO₂R⁶;

R¹, R², R³ and R⁴ are independently selected from H, alkyl, alkenyl, alkynyl, heteroalkyl, cycloalkyl, cycloalkenyl, heterocycloalkyl, aryl and heteroaryl, wherein said alkyl, alkenyl, alkynyl, heteroalkyl, cycloalkyl, cycloalkenyl, heterocycloalkyl, aryl, and heteroaryl are optionally substituted with one or more groups independently selected from alkyl, alkenyl, alkynyl, F, Cl, Br, I, CN, OR⁶, NR⁶R⁷, CC═O)R⁶, C(═O)OR⁶, OC(═O)R⁶, CC═O)NR⁶R⁷, (C₁-C₆ alkyl)amino, CH₃OCH₂O—, R⁶OCC═O)CH═CH₂—, NR⁶SO₂R⁷, SR⁶ and SO₂R⁶,

or R¹ and R² together with the atom to which they are attached form a saturated or partially unsaturated carbocyclic ring, wherein said carbocyclic ring is optionally substituted with one or more groups independently selected from alkyl, alkenyl, alkynyl, F, Cl, Br, I, CN, OR⁶. NR⁶R⁷, C(═O)R⁶, CC═O)OR⁶, OC(═O)R⁶, CC═O)NR⁶R⁷, CCi-C₆ alkyl)amino, CH₃OCH₂O—, R⁶OCC═O)CH═CH₂—, NR⁶SO₂R⁷, SR⁶ and SO₂R⁶;

or R³ and R⁴ together are oxo;

each R⁵ is independently selected from H, F, Cl, Br, I, OMe, CH₃, CH₂F, CHF₂, CF₃ and CF₂CF₃;

R⁶ and R⁷ are independently selected from H, alkyl, alkenyl, alkynyl, heteroalkyl, cycloalkyl, cycloalkenyl, heterocycloalkyl, aryl, and heteroaryl, wherein said alkyl, alkenyl, alkynyl, heteroalkyl, cycloalkyl, cycloalkenyl, heterocycloalkyl, aryl, and heteroaryl are optionally substituted with one or more groups independently selected from alkyl, alkenyl, alkynyl, F, Cl, Br, I, CN, OR⁶, NR⁶R⁷, CC═O)R⁶, C(═O)OR⁶, OC(═O)R⁶, CC═O)NR⁶R⁷, (C₁-C₆ alkyl)amino, CH₃OCH₂O—, R⁶OC(═O)CH═CH₂—, NR⁶SO₂R⁷, SR⁶ and SO₂R⁶;

or R⁶ and R⁷ together with the atom to which they are attached form a saturated or partially unsaturated heterocyclic ring, wherein the heterocyclic ring is optionally substituted with one or more groups independently selected from alkyl, alkenyl, alkynyl, F, Cl, Br, I, CN, OR⁶, NR⁶R⁷, CC═O)R⁶, C(═O)OR⁶, OC(═O)R⁶, C(═O)NR⁶R⁷, (C₁-C₆alkyl)amino, CH₃OCH₂O—, R⁶OC(═O)CH═CH₂—, NR⁶SO₂R⁷, SR⁶ and SO₂R⁶; and n is 0, 1, 2, 3 or 4. WO2007040840A2, the disclosure of which is incorporated by reference in its entirety.

In some embodiments, the immunotherapeutic is a TLR modulator (e.g., TLR7 and/or TLR8 agonist) that is represented by structure of Formula (VI):

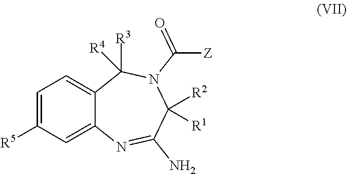

and metabolites, solvates, tautomers, and pharmaceutically acceptable salts and prodrugs thereof, wherein:

Z is H, alkyl, alkenyl, alkynyl, heteroalkyl, cycloalkyl, heterocycloalkyl, aryl, heteroaryl, OR⁶ or NR⁶R⁷, wherein the alkyl, alkenyl, alkynyl, heteroalkyl, cycloalkyl, heterocycloalkyl, aryl and heteroaryl are optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OCC═O)CH═CH₂—, NR⁶SO₂R⁷, SR⁶ and SO₂R⁶;

R¹, R², R³ and R⁴ are independently selected from H, alkyl, alkenyl, alkynyl, heteroalkyl, cycloalkyl, cycloalkenyl, heterocycloalkyl, aryl and heteroaryl, wherein said alkyl, alkenyl, alkynyl, heteroalkyl, cycloalkyl, cycloalkenyl, heterocycloalkyl, aryl, and heteroaryl are optionally substituted with one or more groups independently selected from alkyl, alkenyl, alkynyl, F, Cl, Br, I₉ CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OCC═O)CH═CH₂—, NR⁶SO₂R⁷, SR⁶ and SO₂R⁶,

or R¹ and R² together with the atom to which they are attached form a saturated or partially unsaturated carbocyclic ring, wherein said carbocyclic ring is optionally substituted with one or more groups independently selected from alkyl, alkenyl, alkynyl, F, Cl, Br, I, CN, OR⁶, NR⁶R⁷, C(═O)R⁶. C(═O)OR⁶, OC(═O)R⁶, C(═O)NR⁶R⁷, (C₁-C₆ alkyl)amino, CH₃OCH₂O—, R⁶OC(═O)CH═CH₂—, NR⁶SO₂R⁷, SR⁶ and SO₂R⁶,

or R³ and R⁴ together are oxo;

R⁵ is H, F, Cl, Br, I, OMe, CH₃, CH₂F, CHF₂, CF₃ or CF₂CF₃;

R⁶ and R⁷ are independently selected from H, alkyl, alkenyl, alkynyl, heteroalkyl, cycloalkyl, cycloalkenyl, heterocycloalkyl, aryl, and heteroaryl, wherein said alkyl, alkenyl, alkynyl, heteroalkyl, cycloalkyl, cycloalkenyl, heterocycloalkyl, aryl, and heteroaryl are optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino₅ CH₃OCH₂O—, R⁶OC(═O)CH═CH₂—, NR⁶SO₂R⁷, SR⁶ and SO₂R⁶;

or R⁶ and R⁷ together with the atom to which they are attached form a saturated or partially unsaturated heterocyclic ring, wherein said heterocyclic ring is optionally substituted with one or more groups independently selected from alkyl, alkenyl, alkynyl, F, Cl, Br, I, CN, OR⁶, NR⁶R⁷, C(═O)R⁶, C(═))OR⁶, OC(═O)R⁶, C(═O)NR⁶R⁷, (C₁-C₆ alkyl)ammo, CH₃OCH₂O—, R⁶OC(═O)CH═CH₂—, NR⁶SO₂R⁷, SR⁶ and SO₂R⁶; and

n is O, 1, 2, 3 or 4.

In some embodiments, Z is OR⁶. In some embodiments, R⁶ is alkyl, such as (C₁-6)alkyl. In particular embodiments, R⁶ is ethyl, propyl, isopropyl or isobutyl.

In some embodiments, Z is NR⁶R⁷. In some embodiments, R⁶ and R⁷ are independently H or alkyl, such as (C₁₋₆)alkyl. In some embodiments, R⁶ and R⁷ are ethyl. In some embodiments, n is O or 1.

In some embodiments, R⁵ is C F₂CF₃. In certain embodiments, R³ is H or alkyl, such as (C₁₋₄)alkyl, and R⁴ is H. In certain embodiments, R is alkyl, such as (C₁₋₄)alkyl. In some embodiments, R is methyl. In other particular embodiments, R³ is H. In some embodiments, R is H or alkyl, such as (C₁₋₄)alkyl and R is H. In some embodiments, R¹ is alkyl. In some embodiments, R¹ is methyl. In some particular embodiments, R¹ is H.

In some embodiments, the TLR7 and/or TLR8 agonist that is represented by structure of Formula (XV):

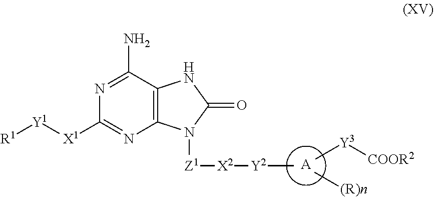

wherein ring A represents a 6-10 membered aromatic carbocyclic ring or a 5-10 membered heteroaromatic ring;
R represents a halogen atom, an alkyl group, a hydroxyalkyl group, a haloalkyl group, an alkoxy group, a hydroxyalkoxy group, a haloalkoxy group, amino group, an alkylamino group, a dialkylamino group, or a 4-7 membered cyclic group containing in the ring 1-2 hetero atoms selected from 1-2 nitrogen atoms and optionally 0-1 oxygen atom or 0-1 sulfur atom;
n represents an integer of 0-2, and when n is 2, the Rs may be the same or different;
Z¹ represents a substituted or unsubstituted alkylene group or a substituted or unsubstituted cycloalkylene group;
X² represents oxygen atom, sulfur atom, SO₂, NR⁵, CO, CONR⁵, NR⁵CO, SO₂NR⁵, NR⁵SO₂, NR⁵CONR⁶ or NR⁵CSNR⁶ (in which R⁵ and R⁶ are each independently hydrogen atom, a substituted or unsubstituted alkyl group, or a substituted or unsubstituted cycloalkyl group);
Y¹, Y² and Y³ represent each independently a single bond or an alkylene group;
X¹ represents oxygen atom, sulfur atom, SO₂, NR⁴ (wherein R⁴ is hydrogen atom or an alkyl group) or a single bond;
R² represents hydrogen atom, a substituted or unsubstituted alkyl group, a substituted or unsubstituted alkenyl group, a substituted or unsubstituted alkynyl group or a substituted or unsubstituted cycloalkyl group; and
R¹ represents hydrogen atom, hydroxy group, an alkoxy group, an alkoxycarbonyl group, a haloalkyl group, a haloalkoxy group, a substituted or unsubstituted aryl group, a substituted or unsubstituted heteroaryl group or a substituted or unsubstituted cycloalkyl group. The linker is linked to one of the possible linking site of the angonist, such as to —NH₂.

In some embodiments, R¹ represents hydrogen, hydroxyl, or a C₁-C₆alkoxy, C₂-C₅alkoxycarbonyl, C₁-C₆ haloalkyl, C₁-C₆ haloalkoxy, C₆-C₁₀ aryl, C₅-C₁₀heteroaryl or C₃-C₈ cycloalkyl group, each group being optionally substituted by one or more substituents independently selected from halogen, hydroxyl, a C₁-C₆alkyl, —C₁-C₆haloalkyl, —C₁-C₆alkoxy, C₁-C₆haloalkoxy, —C₂-C₅ alkoxycarbonyl, amino (NH₂), (mono)-C₁-C₆alkylamino and (di)-C₁-C₆ alkylamino group;

Y¹ represents a single bond or C₁-C₆ alkylene;
X¹ represents a single bond, an oxygen, sulphur atom, sulphonyl (SO₂) or NR₃;
Z¹ represents a C₂-C₆ alkylene or C₃-C₈ cycloalkylene group, each group being optionally substituted by at least one hydroxyl;
X² represents NR⁴;
Y² represents a single bond or C₁-C₆ alkylene;
Y³ represents a single bond or C₁-C₆ alkylene;
n is an integer 0, 1 or 2;
R represents halogen or a C₁-C₆alkyl, C₁-C₆hydroxyalkyl, C₁-C₆haloalkyl, C₁-C₆alkoxy, C₁-C₆hydroxyalkoxy, C₁-C₆haloalkoxy, amino (NH₂), (mono)-C₁-C₆alkylamino, (di)-C₁-C₆alkylamino group or a C₃-C₈ saturated heterocyclic ring containing a ring nitrogen atom and optionally one or more further heteroatoms independently selected from nitrogen, oxygen and sulphur, the heterocyclic ring being optionally substituted by one or more substituents independently selected from halogen, hydroxyl, oxo, C₁-C₆ alkyl, C₁-C₆ alkoxy, C₂-C₅ alkylcarbonyl and C₂-C₅alkoxycarbonyl;
R² represents hydrogen or a C₁-C₆ alkyl, C₂-C₆ alkenyl, C₂-C₆ alkynyl or C₃-C₈ cycloalkyl group, each group being optionally substituted by one or more substituents independently selected from halogen, hydroxyl or a C₁-C₆ alkoxy, a C₂-C₁₀ acyloxy, group selected from a C₂₋₅alkylcarbonyloxy group, a C₂-C₅ alkenylcarbonyloxy group, a C₂-C₅alkynylcarbonyloxy group, a C₆-C₉ arylcarbonyloxy group and a C₅-C₉heteroarylcarbonyloxy group, each of which acyloxy groups may be optionally substituted by one or more substituents independently selected from halogen, hydroxyl, C₁-C₃ alkoxy and phenyl providing that the total number of carbon atoms in the acyloxy group does not exceed 10, amino (NH₂), (mono)-C₁-C₆ alkylamino, (di)-C₁-C₆ alkylamino group and a C₃-C₈ saturated heterocyclic ring containing a ring nitrogen atom and optionally one or more further heteroatoms independently selected from nitrogen, oxygen and sulphur, the heterocyclic ring in turn being optionally substituted by one or more substituents independently selected from halogen, hydroxyl, oxo, C₁-C₆alkyl, C₁-C₆alkoxy, C₂-C₅alkylcarbonyl and C₂-C₅ alkoxycarbonyl group;
R³ represents hydrogen or C₁-C₆ alkyl;
R⁴ represents CO₂R⁵, SO₂R⁵, COR⁵, SO₂NR⁶R⁷ and CONR⁶R⁷;
R⁵ independently represents
(i) 3- to 8-membered heterocyclic ring containing 1 or 2 heteroatoms selected from a ring group NR⁸, S(O)ₘ or oxygen, the 3- to 8-membered heterocyclic ring being optionally substituted by one or more substituents independently selected from halogen, hydroxyl or a C₁-C₆ alkyl and C₁-C₆ alkoxy group, or
(ii) a C₆-C₁₀ aryl or C₅-C₁₀ heteroaryl group, each of which may be optionally substituted by one or more substituents independently selected from halogen, cyano, C₁-C₆alkyl, C₁-C₃ haloalkyl, carboxyl, S(O)ₘR⁹, OR¹⁰, CO₂R¹⁰, SO₂NR¹⁰R¹¹, CONR¹⁰R¹¹, NR¹⁰R¹¹, NR¹⁰SO₂R⁹, NR¹⁰CO₂R⁹, NR¹⁰COR⁹, or
(iii) a C₁-C₆alkyl, C₂-C₆alkenyl, C₂-C₆alkynyl or C₃-C₈ cycloalkyl group, each of which may be optionally substituted by one or more substituents independently selected from halogen, CN, C₃-C₈cycloalkyl, S(O)ₚR¹², OR¹³, COR¹³, CO₂R¹³, SO₂NR¹³R¹⁴, CONR¹³R¹⁴, NR¹³R¹⁴, NR¹³SO₂R¹², NR¹³CO₂R¹², NR¹³COR¹², NR¹³SO₂R¹² or a C₆-C₁₀ aryl or C₅-C₁₀ heteroaryl group or a heterocyclic ring, the latter three groups may be optionally substituted by one or more substituents independently selected from C₁-C₆ alkyl (optionally substituted by hydroxy, C₁-C₆ alkoxy, C₁-C₆ alkoxycarbonyl, amino, C₁-C₆ alkylamino, di-C₁-C₆alkylamino, NH₂C(O)—, C₁-C₆ alkylNHC(O), di-C₁-C₆ alkyl NC(O), —OCH₂CH₂OH, pyrrolidinyl, pyrrolidinylcarbonyl, furanyl, piperidyl, methylpiperidyl or phenyl), C₂-C₆alkenyl (optionally substituted by phenyl), halogen, hydroxy, cyano, carboxy, amino, C₁-C₆alkylamino, di-C₁-C₆ alkylamino, NH₂C(O)—, C₁-C₆ alkyl NHC(O)—, di-C₁-C₆ alkyl NC(O), C₁-C₆ alkoxycarbonyl, C₁-C₆alkylsulphonyl, C₁-C₆ alkylcarbonylamino, C₁-C₆alkylcarbonylmethylamino, phenyl (optionally substituted by hydroxy, fluoro or methyl), pyrrolidinyl, pyridyl, piperidinyl, benzothiazolyl or pyrimidinyl;
R⁶ represents hydrogen or a C₁-C₆alkyl, C₂-C₆alkenyl, C₂-C₆alkynyl, C₃-C₈cycloalkyl group or heterocyclic ring, each of which may be optionally substituted by one or more substituents independently selected from halogen, hydroxyl, oxo, cyano, C₁-C₆ alkyl, C₂-C₆ alkenyl, C₂-C₆ alkynyl, C₃-C₈ cycloalkyl, OR¹⁵, S(O)_{q}R¹⁵, CO₂R¹⁶, COR¹⁶, NR¹⁶R¹⁷, CONR¹⁶R¹⁷, NR¹⁶COR¹⁷, NR¹⁶CO₂R¹⁵, SO₂NR¹⁶R¹⁷, NR¹⁶SO₂R¹⁵, or a C₆-C₁₀ aryl or C₅-C₁₀heteroaryl group or heterocyclic ring, the latter three groups being optionally substituted by one or more substituents independently selected from, C₁-C₆ alkyl, C₃-C₈ cycloalkyl, halogen, S(O)_{q}R¹⁵, CO₂R¹⁶, COR¹⁶, hydroxy or cyano; and
R⁷ represents hydrogen, a C₁-C₆alkyl, C₂-C₆ alkenyl, C₂-C₆alkynyl, or C₃-C₈ cycloalkyl group, each group may be optionally substituted by one or more substituents independently selected from halogen, C₃-C₈cycloalkyl, a C₆-C₁₀ aryl or C₅-C₁₀ heteroaryl group, carboxy, cyano, OR¹⁵, hydroxy or NR¹⁸R¹⁹, or
R⁶ and R⁷ together with the nitrogen atom to which they are attached fowl a 3- to 8-membered saturated or partially saturated heterocyclic ring, optionally containing further heteroatoms or heterogroups selected from nitrogen, S(O)ₘ or oxygen, the heterocyclic ring, may be optionally substituted by one or more substituents independently selected from halogen, hydroxyl, carboxyl, cyano, OR²⁰, NR²¹R²², S(O)_{q}R²³, COR²⁴, CO₂R²⁴, NR²⁴R²⁵, CONR²⁴R²⁵, NR²⁴COR²⁵, NR²⁴CO₂R²³, SO₂NR²⁴R²⁵, NR²⁴SO₂R²³, C₆-C₁₀ aryl, C₅-C₁₀ heteroaryl group, heterocyclic ring, C₁-C₆ alkyl, C₂-C₆ alkenyl, C₂-C₆ alkynyl or C₃-C₈cycloalkyl group, the latter seven groups being optionally substituted by one or more substituents independently selected from halogen, hydroxyl, oxo, cyano, OR²⁰, S(O)_{q}R²³, COR²⁴, CO₂R²⁴, NR²⁴R²⁵, CONR²⁴R²⁵, NR²⁴CO₂R²³, NR²⁴COR²⁵, SO₂NR²⁴R²⁵, NR²⁴SO₂R²³, a heterocyclic ring or a C₆-C₁₁ aryl or C₅-C₁₀heteroaryl group, the latter three groups being optionally substituted by one or more substituents independently selected from C₁-C₆ alkyl, halogen, hydroxy or cyano;
R⁸ represents hydrogen, CO₂R²⁶, COR²⁶, SO₂R²⁶, C₁-C₆ alkyl or C₃-C₆cycloalkyl group, each group may be optionally substituted by one or more substituents independently selected from halogen, hydroxyl, and NR²⁷R²⁸;
R¹⁰, R¹¹, R¹⁶, R¹⁷, R¹⁸, R¹⁹, R²¹, R²², R²⁶, R²⁷ or R²⁸ each independently represents hydrogen, and a C₁-C₆ alkyl or C₃-C₆cycloalkyl group;
R²⁴ and R²⁵ each independently represents hydrogen, and a C₁-C₆ alkyl or C₃-C₆ cycloalkyl group; or
R²⁴ and R²⁵ together with the nitrogen atom to which they are attached form a 3- to 8-membered saturated or partially saturated heterocyclic ring, optionally containing further heteroatoms or heterogroups selected from nitrogen, S(O)ₘ or oxygen;
R⁹, R¹², R¹⁵ and R²³ represent C₁-C₆ alkyl or C₃-C₆ cycloalkyl;
R¹³ and R¹⁴ are defined as for R⁶ and R⁷ respectively;
R²⁰ represents a C₁-C₆ alkyl optionally substituted by one or more substituents independently selected from halogen, hydroxyl or OR²³;
m, p, q and r each independently represent an integer 0, 1 or 2; and
A represents a C₆-C₁₀ aryl or C₅-C₂ heteroaryl group. See WO2008004948A1, U.S. Pat. Nos. 8,138,172, and 8,575,180, the disclosures of which are incorporated by reference in their entireties.

In some embodiments, the TLR7 and/or TLR8 agonist having the structure of:

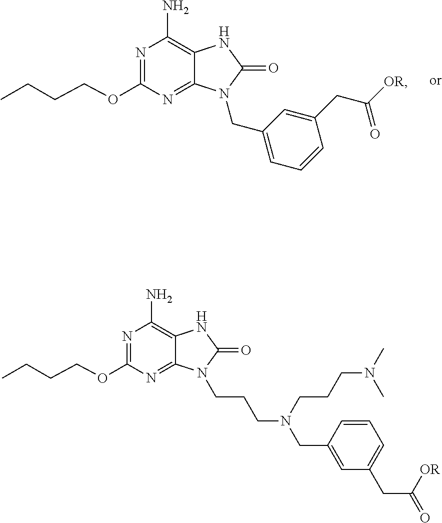

wherein R is Me or H.

In some embodiments, the TLR7 and/or TLR8 agonist having the structure of:

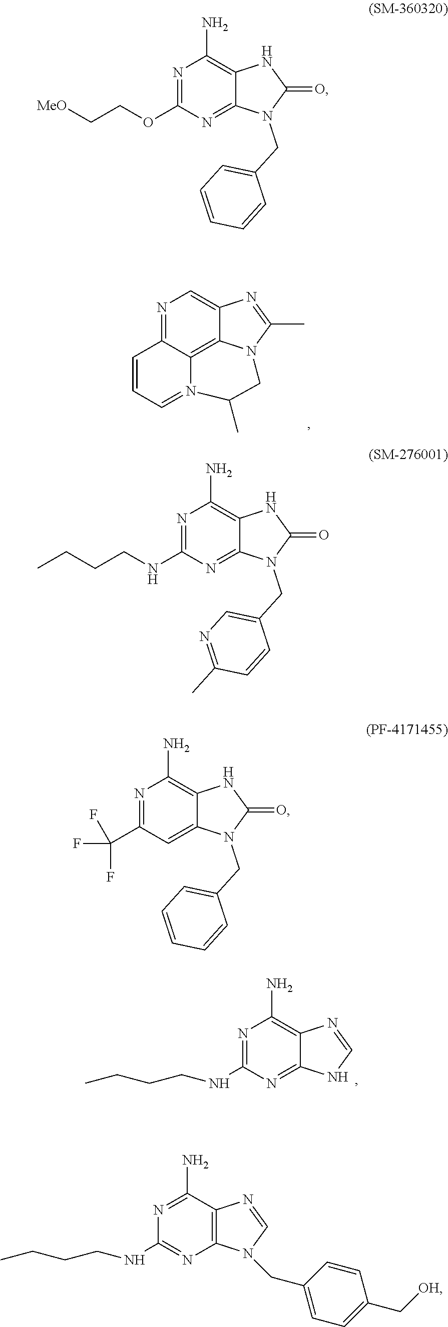

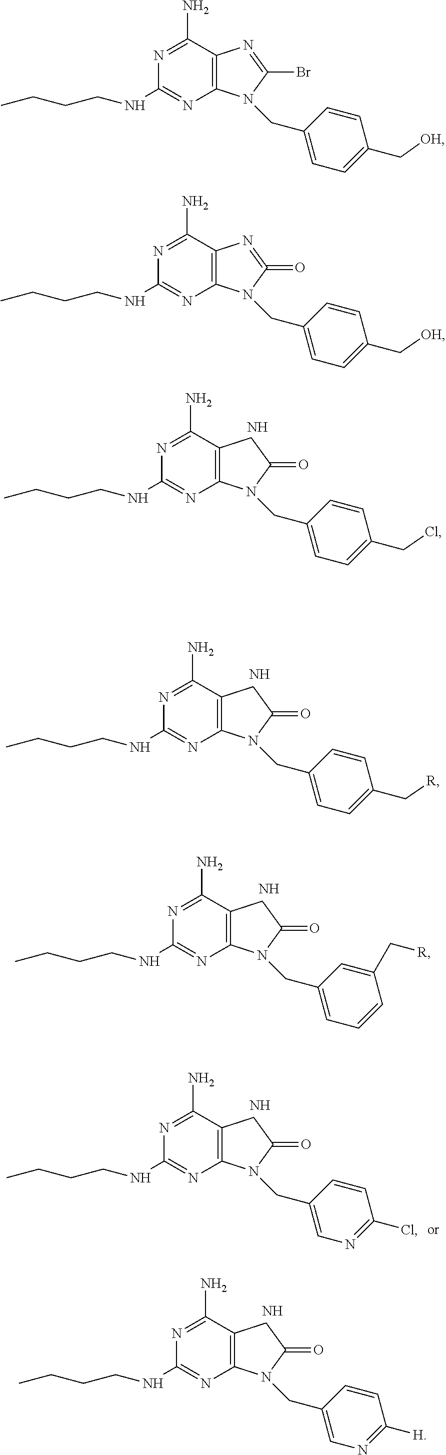

In some embodiments, the TLR7 and/or TLR8 agonist having the structure of Formula (XVI):

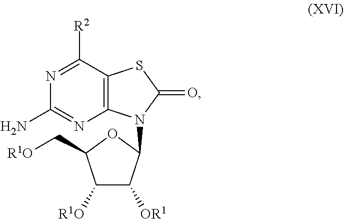

wherein: R¹ is independently H, —C(O)R³, or a racemic, L-, or D-amino acid group —C(O)CHNH₂R⁴, wherein R³ is a substituted or unsubstituted alkyl, and R⁴ is H, or a substituted or unsubstituted alkyl;
R² is H, O, OR⁵, or N(R⁶)₂, wherein R⁵ is independently H or alkyl, and wherein R⁶ is independently H, substituted or unsubstituted alkyl, cycloalkyl, or together with nitrogen forms a substituted or unsubstituted heterocycloalkyl ring; and wherein if R is —OH, at least one of the R groups is a racemic, L-, or D-amino acid group —C(O)CHNH₂R⁴. See U.S. Pat. No. 6,924,271, the disclosure of which is incorporated by reference in its entirety.

In some embodiments, at least one of the R¹ groups is a racemic, L-, or D-amino acid group —C(O)CHNH₂R⁴, wherein R⁴ is a substituted or unsubstituted alkyl, and wherein the remaining R¹ groups are H; R² is OR⁵ or N(R⁶)₂, wherein R⁵ is independently selected from H or alkyl, and wherein R is independently H, substituted or unsubstituted alkyl, cycloalkyl, or together with nitrogen forms a substituted or unsubstituted heterocycloalkyl ring.

In some embodiments, at least one of the R¹ groups is a L-amino acid group —C(O)CHNH₂R⁴, wherein R⁴ is a substituted or unsubstituted alkyl, and wherein the remaining R¹ groups are H; R² is OR⁵ or N(R⁶)₂, wherein R⁴ is a substituted alkyl, and wherein R⁶ is independently H or substituted or unsubstituted alkyl.

In some embodiments, at least one of the R¹ groups is a L-amino acid group —C(O)CHNH₂R, wherein R⁴ is —CH(CH₃)₂, and wherein the remaining R¹ groups are H; and R² is OH.

In some embodiments, the TLR7 and/or agonist is selected from the group consisting of:

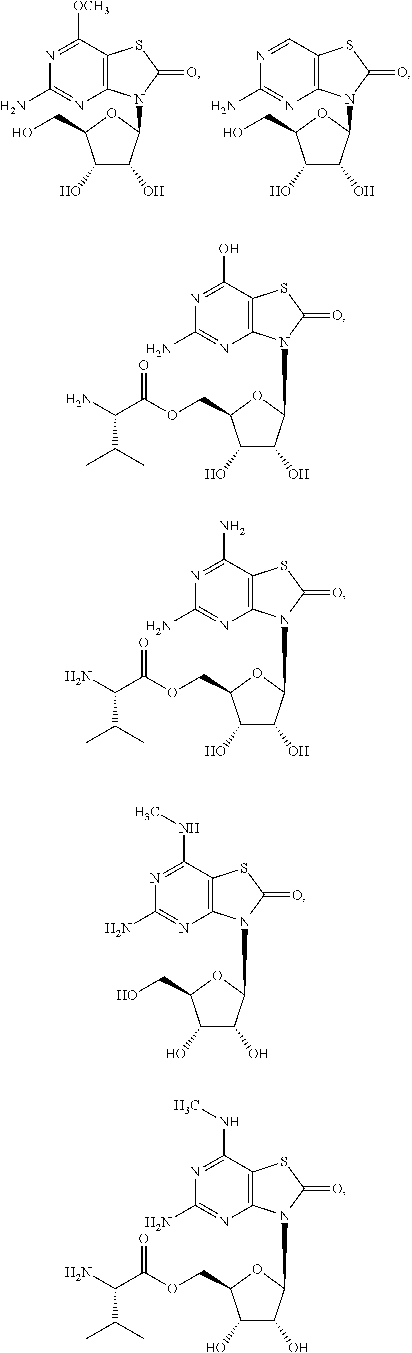 [SMILES: COc1nc(N)nc2c1sc(=O)n2[C@@H]1O[C@H](CO)[C@@H](O)[C@H]1O ; Nc1ncc2sc(=O)n([C@@H]3O[C@H](CO)[C@@H](O)[C@H]3O)c2n1 ; CC(C)[C@H](N)C(=O)OC[C@H]1O[C@@H](n2c(=O)sc3c(O)nc(N)nc32)[C@H](O)[C@@H]1O ; CC(C)[C@H](N)C(=O)OC[C@H]1O[C@@H](n2c(=O)sc3c(N)nc(N)nc32)[C@H](O)[C@@H]1O ; CNc1nc(N)nc2c1sc(=O)n2[C@@H]1O[C@H](CO)[C@@H](O)[C@H]1O ; CNc1nc(N)nc2c1sc(=O)n2[C@@H]1O[C@H](COC(=O)[C@@H](N)C(C)C)[C@@H](O)[C@H]1O]

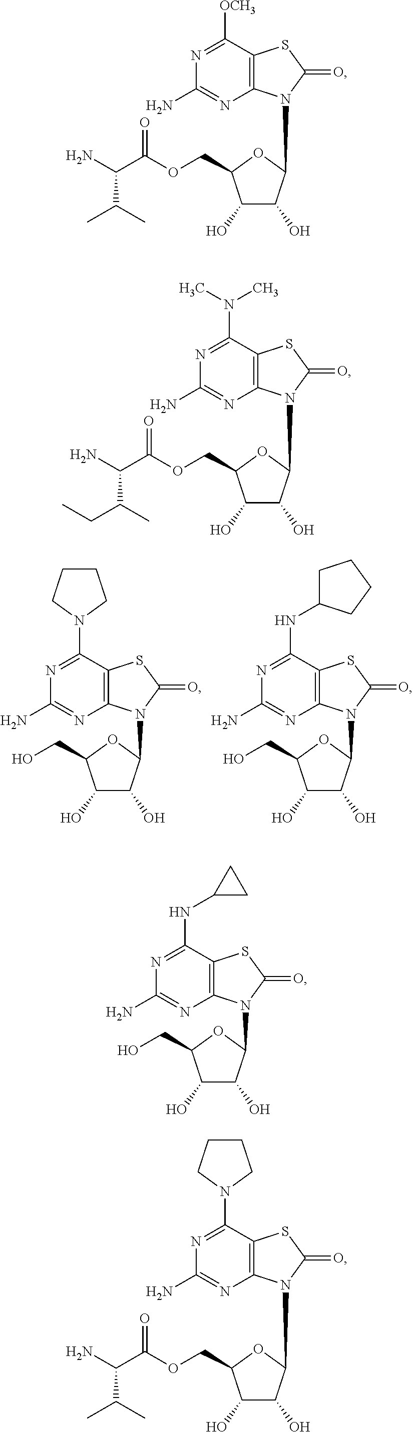

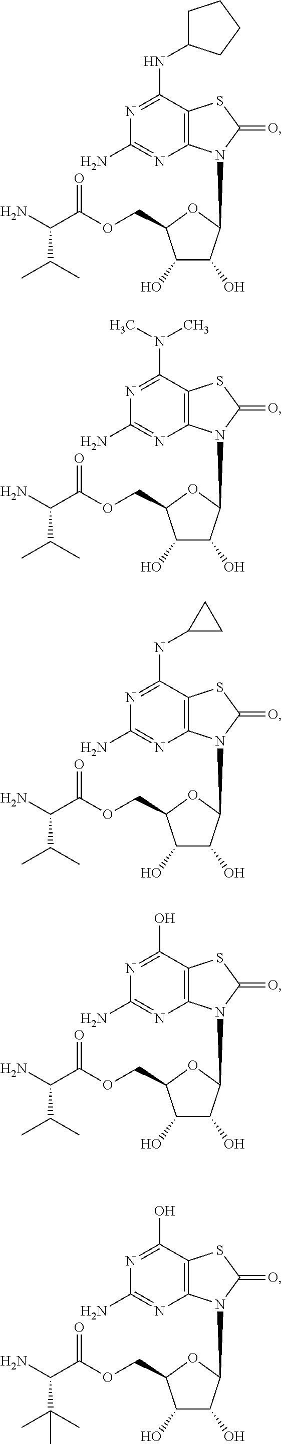

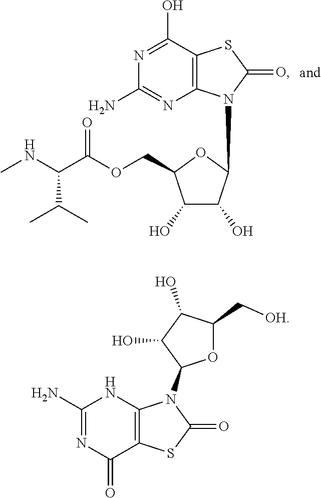 [SMILES: CN[C@H](C(=O)OC[C@H]1O[C@@H](n2c(=O)sc3c(O)nc(N)nc32)[C@H](O)[C@@H]1O)C(C)C ; Nc1nc(=O)c2sc(=O)n([C@@H]3O[C@H](CO)[C@@H](O)[C@H]3O)c2[nH]1]

In some embodiments, the TLR7 and/or TLR8 agonist having the structure of:

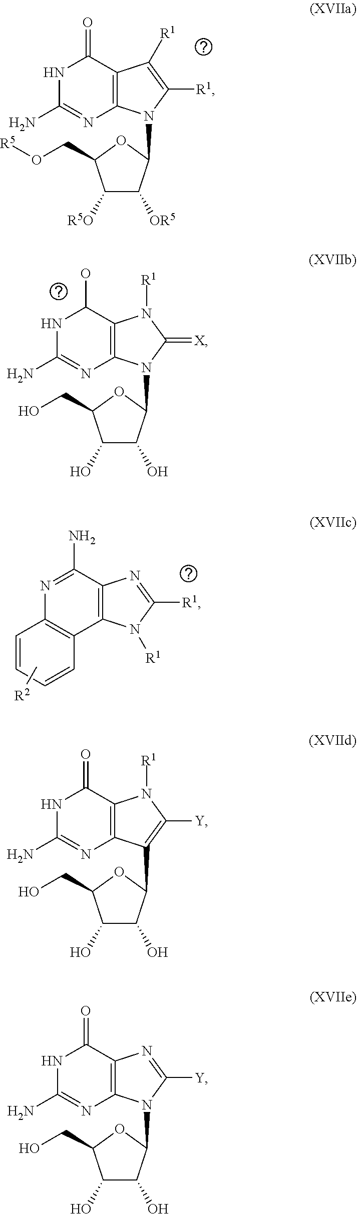

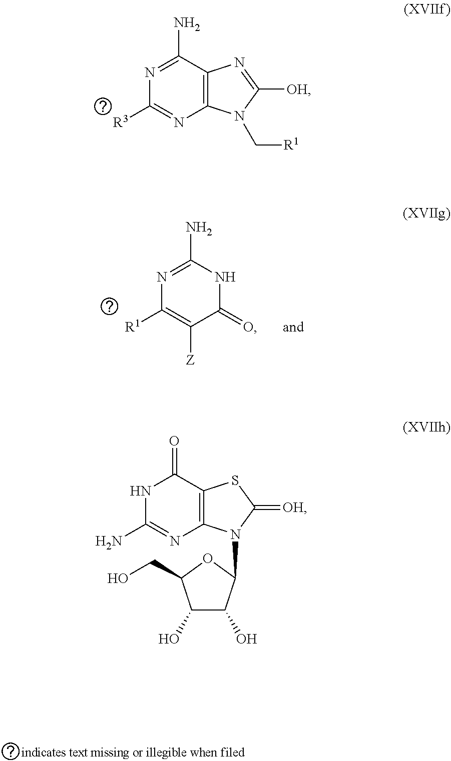

wherein:

each R¹ is H, or a substituted or unsubstituted alkyl, alkenyl, or alkynyl, which may be interrupted by one or more O, S, or N heteroatoms, or a substituted or unsubstituted aryl or heteroaryl;

R² is H, OH, SH, halo, or a substituted or unsubstituted alkyl, alkenyl, or alkynyl, which may be interrupted by one or more O, S, or N heteroatoms, or a substituted or unsubstituted —O-(alkyl), —O-(aryl), —O-(heteroaryl), —S-(alkyl), —S-(aryl), —S-(heteroaryl), aryl, or heteroaryl;

R³ is H, OH, or SH, or a substituted or unsubstituted alkyl, alkenyl, alkynyl, aryl, heteroaryl, —O-(alkyl), —O-(aryl), —O-(heteroaryl), —S-(alkyl), —S-(aryl), —S-(heteroaryl), —NH(alkyl), —NH(aryl), —NH(heteroaryl), —NH(R⁴)(alkyl), —NH(R⁴)(aryl), or —NH(R⁴)(heteroaryl), wherein R⁴ is a substituted or unsubstituted alkyl;

X is O or S:

Y is H, halo, OH, OR⁴, SH, SR⁴, or a substituted or unsubstituted alkyl or aryl; and

Z is H, halo, OH, OR⁴, SH, or SR⁴. See U.S. Pat. No. 7,576,068, the disclosure of which is incorporated by reference in its entirety.

In some embodiments, the TLR7 and/or TLR8 agonist having the structure of Formula (XVIII):

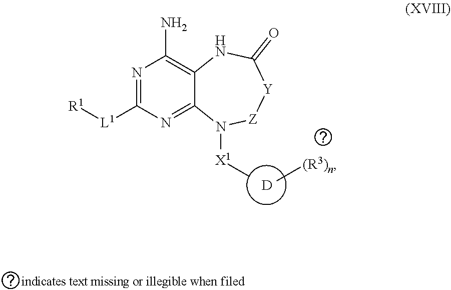

wherein:
Y—Z is —CR⁴R⁵—, —CR⁴R⁵—CR⁴R⁵—, —C(O)CR⁴R⁵—, —CR⁴R⁵C(O)—, —NR⁸C(O)—, —C(O)NR⁸—, —CR⁴R⁵S(O)₂—, or —CR⁵═CR⁵—;
L¹ is —NR⁸—, —O—, —S—, —N(R⁸)C(O)—, —S(O)₂—, —S(O)—C(O)N(R⁸)—, —N(R⁸)S(O)₂—, —S(O)₂N(R⁸)— or a covalent bond;
R¹ is alkyl, substituted alkyl, haloalkyl, alkenyl, substituted alkenyl, alkynyl, substituted alkynyl, heteroalkyl, substituted heteroalkyl, carbocyclyl, substituted carbocyclyl, carbocyclylalkyl, substituted carbocyclylalkyl, heterocyclyl, substituted heterocyclyl, heterocyclylalkyl, or substituted heterocyclylalkyl, arylalkyl, substituted arylalkyl, heteroarylalkyl, substituted heteroarylalkyl, carbocyclylheteroalkyl, substituted carbocyclylheteroalkyl, heterocyclylheteroalkyl, substituted heterocyclylheteroalkyl, arylheteroalkyl, substituted arylheteroalkyl, heteroarylheteroalkyl, or substituted heteroarylheteroalkyl;
X¹ is alkylene, substituted alkylene, heteroalkylene, substituted heteroalkylene, alkenylene, substituted alkenylene, alkynylene, substituted alkynylene, carbocyclylene, substituted carbocyclylene, heterocyclylene, substituted heterocyclylene, —NR⁸—, —O—, —C(O)—, —S(O)—, S(O)₂—, or a bond.
D is carbocyclyl, substituted carbocyclyl, heterocyclyl or substituted heterocyclyl wherein said carbocyclyl, substituted carbocyclyl, heterocyclyl or substituted heterocyclyl is substituted with one or two -L²-NR⁶R⁷; or
D is a heterocyclyl, substituted heterocyclyl, heteroaryl or substituted heteroaryl wherein said heterocyclyl, substituted heterocyclyl, heteroaryl or substituted heteroaryl comprises one to four nitrogen atoms;
each L² is independently alkylene, substituted alkylene, heteroalkylene, substituted heteroalkylene, or a covalent bond;
each R³ is independently halogen, cyano, azido, nitro, alkyl, substituted alkyl, hydroxyl, amino, heteroalkyl, substituted heteroalkyl, alkoxy, haloalkyl, haloalkoxy, —CHO, —C(O)OR⁸, —S(O)R⁸, —S(O)₂R⁸; —C(O)NR⁹R¹⁰, —N(R⁹)C(O)R⁸, carbocyclyl, substituted carbocyclyl, carbocyclylalkyl, substituted carbocyclylalkyl, alkenyl, substituted alkenyl, alkynyl, substituted alkynyl, —S(O)₂NR⁹R¹⁰, —N(R⁹)S(O)₂R⁸, —N(R⁹)S(O)₂OR¹⁰, —OS(O)₂NR⁹R¹⁰;
n is 0, 1, 2, 3, 4 or 5:
R⁴ and R⁵ are each independently H, alkyl, substituted alkyl, haloalkyl, heteroalkyl, substituted heteroalkyl, carbocyclyl, substituted carbocyclyl, carbocyclylalkyl, substituted carbocyclylalkyl, heterocyclyl, substituted heterocyclyl, heterocyclylalkyl, substituted heterocyclylalkyl, arylalkyl, substituted arylalkyl, heteroarylalkyl, substituted heteroarylalkyl, carbocyclylheteroalkyl, substituted carbocyclylheteroalkyl, heterocyclylheteroalkyl, substituted heterocyclylheteroalkyl, arylheteroalkyl, substituted arylheteroalkyl, heteroarylheteroalkyl, or substituted heteroarylheteroalkyl, cyano, azido, OR⁸, —C(O)H, —C(O)R⁸, —S(O)R⁸, —S(O)₂R⁸, —C(O)OR⁸, or —C(O)NR⁹R¹⁰; or
R⁴ and R⁵, taken together with the carbon to which they are both attached, form a carbocycle, substituted carbocycle, heterocycle or substituted heterocycle; or
R⁴ and R⁵, when on the same carbon atom, taken together with the carbon to which they are attached are —C(O)— or —C(NR⁸)—; or
two R⁴ or two R⁵ on adjacent carbon atoms when taken together with the carbons to which they are attached form a 3 to 6 membered carbocycle, substituted carbocycle, heterocycle or substituted heterocycle;
R⁶ and R⁷ are each independently H, alkyl, substituted alkyl, alkenyl, substituted alkenyl, alkynyl, substituted alkynyl, haloalkyl, heteroalkyl, substituted heteroalkyl, carbocyclyl, substituted carbocyclyl, carbocyclylalkyl, substituted carbocyclylalkyl, heterocyclyl, substituted heterocyclyl, heterocyclylalkyl, substituted heterocyclylalkyl, arylalkyl, substituted arylalkyl, heteroarylalkyl, substituted heteroarylalkyl, carbocyclylheteroalkyl, substituted carbocyclylheteroalkyl, heterocyclylheteroalkyl, substituted heterocyclylheteroalkyl, arylheteroalkyl, substituted arylheteroalkyl, heteroarylheteroalkyl, or substituted heteroarylheteroalkyl, —C(O)H, —C(O)R⁸, —S(O)R⁸, —S(O)₂R₈, —C(O)OR⁸, or —C(O)NR⁹R¹⁰, S(O)₂NR⁹R¹⁰; or
R⁶ and R⁷, taken together with the nitrogen to which they are both attached, form a substituted or unsubstituted heterocycle, which may contain one or more additional heteroatoms selected from N, O, P, or S; or
R⁷ taken together with L², and the N to which they are both attached, forms a substituted or unsubstituted 3 to 8 membered heterocycle which may contain one or more additional heteroatoms selected from N, O, S, or P;
R⁸ is H, alkyl, substituted alkyl, haloalkyl, alkenyl, substituted alkenyl, alkynyl, substituted alkynyl, heteroalkyl, substituted heteroalkyl, carbocyclyl, substituted carbocyclyl, carbocyclylalkyl, substituted carbocychylalkyl, heterocyclyl, substituted heterocyclyl, heterocyclylalkyl, substituted heterocyclylalkyl, arylalkyl, substituted arylalkyl, heteroarylalkyl, substituted heteroarylalkyl, carbocyclylheteroalkyl, substituted carbocyclylheteroalkyl, heterocyclylheteroalkyl, substituted heterocyclylheteroalkyl, arylheteroalkyl, substituted arylheteroalkyl, heteroarylheteroalkyl, or substituted heteroarylheteroalkyl; and
R⁹ and R¹⁰ are each independently H, alkyl, substituted alkyl, alkenyl, substituted alkenyl, alkynyl, substituted alkynyl, haloalkyl, heteroalkyl, substituted heteroalkyl, carbocyclyl, substituted carbocyclyl, carbocyclylalkyl, substituted carbocyclylalkyl, heterocyclyl, substituted heterocyclyl, heterocyclylalkyl, substituted heterocyclylalkyl, arylalkyl, substituted arylalkyl, heteroarylalkyl, substituted heteroarylalkyl, carbocyclylheteroalkyl, substituted carbocyclylheteroalkyl, heterocyclheteroalkyl, substituted heterocyclylheteroalkyl, arylheteroalkyl, substituted arylheteroalkyl, heteroarylheteroalkyl, or substituted heteroarylheteroalkyl; or
R⁹ and R¹⁰, taken together with the nitrogen to which they are both bonded, form a substituted or unsubstituted heterocycle;
wherein each substituted alkyl, substituted alkenyl, substituted alkynyl, substituted heteroalkyl, substituted carbocyclyl, substituted carbocyclylalkyl, substituted heterocyclyl, substituted heterocychylalkyl, substituted arylalkyl, substituted heteroarylalkyl, substituted carbocyclylheteroalkyl, substituted heterocyclylheteroalkyl, substituted arylheteroalkyl, substituted heteroarylheteroalkyl, substituted alkylene, substituted heteroalkylene, substituted alkenylene, substituted alkynylene, substituted carbocyclylene, or substituted heterocyclylene is independently substituted with one to four substituents selected from the group consisting of -halogen, —R, —O⁻, ═O, —OR, —SR, —S⁻, —NR₂, —N(+)R₃, ═NR, —C(halogen)₃, —CR(halogen)₂, —CR₂(halogen), —CN, —OCN, —SCN, —N═C═O, —NCS, —NO, —NO₂, ═N₂,
—N₃, —NRC(═O)R, —NRC(═O)OR, —NRC(═O)NRR, —C(═O)NRR, —C(═O)OR, —OC(═O)NRR, —OC(═O)OR, —C(═O)R, —S(═O)₂OR, —S(═O)₂R, —OS(═O)₂OR, —S(═O)₂NR, —S(═O)R,
—NRS(═O)₂R, —NRS(═O)₂NRR, —NRS(═O)₂OR, —OP(═O)(OR)₂, —P(═O)(OR)₂, —P(O)(OR)(O)R, —C(═O)R, —C(═S)R, —C(═O)OR, —C(═S)OR, —C(═O)SR, —C(═S)SR, —C(═O)NRR, —C(═S)NRR, —C(═NR)NRR, and —NRC(═NR)NRR; wherein each R is independently H, alkyl, cycloalkyl, aryl, arylalkyl, or heterocyclyl. See US 20100143301 A1, the disclosure of which is incorporated by reference in its entirety.

In some embodiments, the TLR7 and/or TLR8 agonist having the structure of:

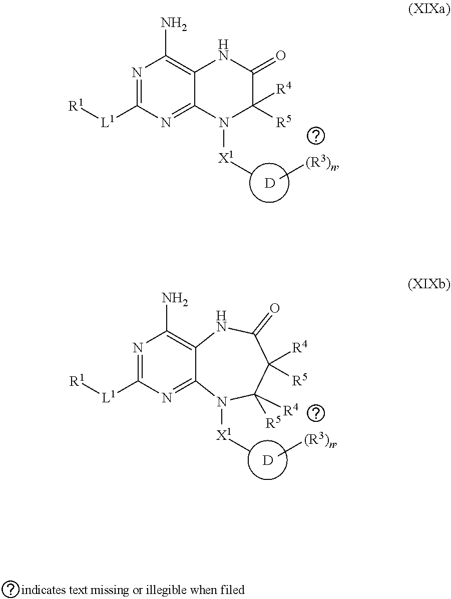

wherein:

### L¹ is —NH— or —O—;

R¹ is alkyl, substituted alkyl, heteroalkyl, substituted heteroalkyl, heterocyclylalkyl, substituted heterocyclylalkyl, carbocyclylalkyl or substituted carbocyclylalkyl;
each of R⁴ and R⁵ independently is H or C₁-C₆ alkyl or R⁴ and R⁵ taken together with the carbon to which they are attached is —C(O)—;
X¹ is C₁-C₆ alkylene, C₁-C₆ heteroalkylene or C₁-C₆ substituted heteroalkylene;
D is phenyl, biphenyl or pyridinyl, wherein said phenyl, biphenyl or pyridinyl is substituted with -L²-NR⁶R⁷; or
D is pyridinyl, piperidinyl, piperazinyl or 1,2,3,4-tetrahydroisoquinolinyl;
n is 0 or 1;
R³ is halogen, cyano, alkyl, carbocyclyl, carbocyclylalkyl, haloalkyl, —C(O)OR⁶, —C(O)NR⁹R¹⁰ or —CHO;
L² is C₁-C₆ alkylene or a covalent bond;
each of R⁶ and R⁷ independently is H, alkyl, or heteroaryl; or
R⁶ and R⁷ taken together with the nitrogen to which they are attached form a substituted or unsubstituted 4-6 membered heterocycle comprising 0 to 2 heteroatoms selected from N, O or S.

In some embodiments, the TLR7 and/or TLR8 agonist having the structure of:

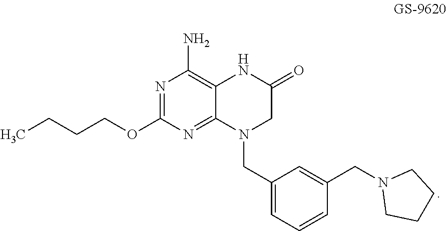 [SMILES: CCCCOc1nc(N)c2c(n1)N(Cc1cccc(CN3CCCC3)c1)CC(=O)N2]

### C. Amount of Immunotherapeutics in the Therapeutic Combinations

In another aspect, the present disclosure provides a therapeutic combination comprising an immune modulatory chemotherapeutic and an immunotherapeutic in an amount that is suitable for the combination therapy treatment of diseases such as tumors and cancers.

In some embodiments, the immunotherapeutic is of an amount that is capable of: (1) inducing IFN-α in an enriched human blood DCs; (2) inducing TNF-α in an enriched human blood DCs; and/or (3) inducing IL-12-α in an enriched human blood DCs.

Methods for measuring the activity of the immunotherapeutics are: 1) an assay to measure cytokines release from human dendritic cell stimulated by immunotherapy; and 2) an efficacy study in a tumor model treated by immunotherapy.

In some embodiments, the immunotherapeutic (e.g. resiquimod or its analogues) is adminstered, either orally or intravenously using oral formulation or intravenous formulation, of an amount so that the local concentration of the immunotherapetuics (e.g. near or at the tumor site of a solid tumor) is from about 0.005 μg/ml to about 1, 2, 3, 4, 5, 6, 7, 8, 9, 10, 11, or 12 μg/ml, or a range bound by any pair of values in the preceding list (all inclusive).

The local concentration of the immunotherapetuic (e.g. near or at the tumor site of a solid tumor) can measured using methods known in the art, such as measuring the tissue or serum concentration. Local effective concentration of therapeutic agent is depended on its absorption from various routes, tissue distribution, and metabolism process, and plasma pharmacokinetics of agent and tissue concentration could be measured routinely using methods known in the art.

In some embodiments, the immunotherapeutic is adminstered of an amount so that the local concentration of the immunotherapetuic (e.g. near or at the tumor site of a solid tumor) is from about 0.05 μg/ml, 0.1 μg/ml, 0.15 μg/ml, 0.2 μg/ml, 0.3 μg/ml, or 0.4 μg/ml, to about 0.5 μg/ml, or a range bound by any pair of values in the preceding list (all inclusive).

In some embodiments, the subject is administered an oral formulation comprising the immunotherapeutic (e.g. resiquimod or its analogues) in a dose of from about 0.0005 mg/kg, 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, or 0.015 mg/kg, to about 0.02 mg/kg, or a range bound by any pair of values in the preceding list (all inclusive), two times per week. In some embodiments, the subject is administered an oral formulation comprising the immunotherapeutic (e.g. resiquimod or its analogues) in a dose of from about 0.0005 mg/kg, to about 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.015 mg/kg, or 0.02 mg/kg, or a range bound by any pair of values in the preceding list (all inclusive), two times per week.

In some embodiments, the subject is administered an oral formulation comprising the immunotherapeutic (e.g. resiquimod or its analogues) in a dose of at least 0.0001 mg/kg but less than or about 0.0005 mg/kg, 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, or 0.01 mg/kg, two times per week.

In some embodiments, the subject is administered an intravenous formulation comprising the immunotherapeutic (e.g. resiquimod or its analogues) in a dose of from about 0.0005 mg/kg, 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, or about 0.015 mg/kg, to about 0.02 mg/kg, or a range bound by any pair of values in the preceding list (inclusive), weekly. In some embodiments, the subject is administered an intravenous formulation comprising the immunotherapeutic (e.g. resiquimod or its analogues) in a dose of from about 0.0005 mg/kg, to about 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.015 mg/kg, or 0.02 mg/kg, or a range bound by any pair of values in the preceding list (inclusive), weekly.

In some embodiments, the method comprises administering to said subject an intravenous formulation comprising said immunotherapeutic (e.g. resiquimod or its analogues) in a dose of between about 0.0008 mg/kg and about 0.0133 mg/kg, weekly.

In some embodiments, the subject is administered an intravenous formulation comprising the immunotherapeutic (e.g. resiquimod or its analogues) in a dose of at least 0.0001 mg/kg but less than or about 0.003 mg/kg, 0.004 mg/kg, 0.005 mg/kg, 0.006 mg/kg, or about 0.01 mg/kg, weekly. For references regarding safe dosage of immunotherapeutics, see Jurk et al., Nature Immunology, Vol. 4, No. 6″499 (2002), and Pockros et al., J. Hepatology, 47:174-182 (2007), the disclosure of which is incorporated by reference in their entirety.

### III. Pharmaceutical Formulations and Administration

The present disclosure further relates to a pharmaceutical formulation comprising a compound of the present embodiments or a pharmaceutically acceptable salt thereof, and one or more pharmaceutically acceptable carriers.

The compounds described herein including pharmaceutically acceptable carriers such as addition salts or hydrates thereof, can be delivered to a patient using a wide variety of routes or modes of administration. Suitable routes of administration include, but inhalation, transdermal, oral, rectal, transmucosal, intestinal and parenteral administration, including intramuscular, subcutaneous and intravenous injections. Preferably, the compounds of the present embodiments comprising immune modulators are administered parenterally, more preferably intravenously.

As used herein, the terms “administering” or “administration” are intended to encompass all means for directly and indirectly delivering a compound to its intended site of action.

The compounds described herein, or pharmaceutically acceptable salts and/or hydrates thereof, may be administered singly, in combination with other compounds of the present embodiments, and/or in cocktails combined with other therapeutic agents. Of course, the choice of therapeutic agents that can be co-administered with the compounds of the present disclosure will depend, in part, on the condition being treated.

For example, when administered to patients suffering from a disease state caused by an organism that relies on an autoinducer, the compounds of the present embodiments can be administered in cocktails containing agents used to treat the pain, infection and other symptoms and side effects commonly associated with the disease. Such agents include, e.g., analgesics, antibiotics, etc.

When administered to a patient undergoing cancer treatment, the compounds may be administered in cocktails containing anti-cancer agents and/or supplementary potentiating agents. The compounds may also be administered in cocktails containing agents that treat the side-effects of radiation therapy, such as anti-emetics, radiation protectants, etc.

Supplementary potentiating agents that can be co-administered with the compounds of the present embodiments include, e.g., tricyclic anti-depressant drugs (e.g., imipramine, desipramine, amitriptyline, clomipramine, trimipramine, doxepin, nortriptyline, protriptyline, amoxapine and maprotiline); non-tricyclic and anti-depressant drugs (e.g., sertraline, trazodone and citalopram); Ca+2 antagonists (e.g., verapamil, nifedipine, nitrendipine and caroverine); amphotericin; triparanol analogues (e.g., tamoxifen); antiarrhythmic drugs (e.g., quinidine); antihypertensive drugs (e.g., reserpine); thiol depleters (e.g., buthionine and sulfoximine); and calcium leucovorin.

The active compound(s) of the present embodiments are administered per se or in the form of a pharmaceutical composition wherein the active compound(s) is in admixture with one or more pharmaceutically acceptable carriers, excipients or diluents. Pharmaceutical compositions for use in accordance with the present embodiments are typically formulated in a conventional manner using one or more physiologically acceptable carriers comprising excipients and auxiliaries, which facilitate processing of the active compounds into preparations which, can be used pharmaceutically. Proper formulation is dependent upon the route of administration chosen.

For transmucosal administration, penetrants appropriate to the barrier to be permeated are used in the formulation. Such penetrants are generally known in the art.

For oral administration, the compounds can be formulated readily by combining the active compound(s) with pharmaceutically acceptable carriers well known in the art. Such carriers enable the compounds of the present embodiments to be formulated as tablets, pills, dragees, capsules, liquids, gels, syrups, slurries, and suspensions for oral ingestion by a patient to be treated. Pharmaceutical preparations for oral use can be obtained solid excipient, optionally grinding a resulting mixture, and processing the mixture of granules, after adding suitable auxiliaries, if desired to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxyniethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present embodiments are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Injection is a preferred method of administration for the compositions of the present embodiments. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents may be added, such as the cross-linked polyvinyl pyrrolidone, agar, or alginic acid or a salt thereof such as sodium alginate.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents, which increase the solubility of the compounds to allow for the preparation of highly, concentrated solutions. For injection, the agents of the present embodiments may be formulated in aqueous solutions, preferably in physiologically compatible buffers such as Hanks's solution, Ringer's solution, or physiological saline buffer.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation or transcutaneous delivery (e.g., subcutaneously or intramuscularly), intramuscular injection or a transdermal patch. Thus, for example, the compounds may be formulated with suitable polymeric or hydrophobic materials (e.g., as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include calcium carbonate, calcium phosate, various sugars, starches, cellulose derivatives, gelatin, and polymers such as polyethylene glycols.

In some embodiments, the pharmaceutical composition of the present embodiments further comprise an additional therapeutic agent beyond the chemotherapeutic and immunotherapeutic.

In some embodiments, the additional therapeutic agent is an anticancer agent.

In some embodiments, the additional anticancer agent is selected from an antimetabolite, an inhibitor of topoisomerase I and II, an alkylating agent, a microtubule inhibitor, an antiandrogen agent, a GNRh modulator or mixtures thereof.

In some embodiments, the additional therapeutic agent is a chemotherapeutic agent.

By “chemotherapeutic agent” herein is meant a chemical compound useful in the treatment of cancer. Examples are but not limited to: Gemcitabine, Irinotecan, Doxorubicin, 5-Fluorouracil, Cytosine arabinoside (“Ara-C”), Cyclophosphamide, Thiotepa, Busulfan, Cytoxin, TAXOL, Methotrexate, Cisplatin, Melphalan, Vinblastine and Carboplatin.

In some embodiments, the second chemotherapeutic agent is selected from the group consisting of tamoxifen, raloxifene, anastrozole, exemestane, letrozole, imatinib, paclitaxel, cyclophosphamide, lovastatin, minosine, gemcitabine, cytarabine, 5-fluorouracil, methotrexate, docetaxel, goserelin, vincristine, vinblastine, nocodazole, teniposide etoposide, gemcitabine, epothilone, vinorelbine, camptothecin, daunorubicin, actinomycin D, mitoxantrone, acridine, doxorubicin, epirubicin, or idarubicin.

### IV. Kits

In another aspect, the present embodiments provides kits containing the therapeutic combinations provided herein and directions for using the therapeutic combinations. The kit may also include a container and optionally one or more vial, test tube, flask, bottle, or syringe. Other formats for kits will be apparent to those of skill in the art and are within the scope of the present invention.

### V. Medical Use

In another aspect, the present disclosure provides a method for treating a disease condition in a subject that is in need of such treatment, comprising: administering to the subject a therapeutic combination or pharmaceutical composition comprising a therapeutically effective amount of the compound of the present embodiments or a pharmaceutically acceptable salt thereof, and a pharmaceutical acceptable carrier.

In addition to the compositions and constructs described above, the present embodiments also provide a number of uses of the disclosed combinations. The combinations of the present embodiments comprise an immune modulatory chemotherapeutic that has one or more of the functions of Tumor cell killers, Treg inhibitors, myeloid-derived suppressor cells inhibitors, and NK activators. These uses comprise administering to an animal such as a mammal or a human in need thereof an effective amount of compounds of the present embodiments, that is, administration of the disclosed combinations.

The combinations of the present embodiments are useful for treating diseases such as cancer in a subject, such as a human being. Combinations and uses for treating tumors by providing a subject the composition in a pharmaceutically acceptable manner, with a pharmaceutically effective amount of a composition of the present disclosure are provided.

By “cancer” herein is meant the pathological condition in humans that is characterized by unregulated cell proliferation. Examples include but are not limited to: carcinoma, lymphoma, blastoma, and leukemia. More particular examples of cancers include but are not limited to: Acute myeloid leukemia (AML), Breast cancer, Chronic lymphocytic leukemia (CLL), Chronic myelogenous leukemia (CML), Hodgkin lymphoma, Multiple myeloma, Mycosis fungoides, Neuroblastoma, Non-Hodgkin lymphoma (NHL), Ovarian cancer, and Retinoblastoma.

By “inhibiting” or “treating” or “treatment” herein is meant to reduction, therapeutic treatment and prophylactic or preventative treatment, wherein the objective is to reduce or prevent the aimed pathologic disorder or condition. In one example, following administering of a compound of the present embodiments, a cancer patient may experience a reduction in tumor size. “Treatment” or “treating” includes (1) inhibiting a disease in a subject experiencing or displaying the pathology or symptoms of the disease, (2) ameliorating a disease in a subject that is experiencing or displaying the pathology or symptoms of the disease, and/or (3) affecting any measurable decrease in a disease in a subject or patient that is experiencing or displaying the pathology or symptoms of the disease. To the extent a compound of the present embodiments may prevent growth and/or kill cancer cells, it may be cytostatic and/or cytotoxic.

By “therapeutically effective amount” herein is meant an amount of a compound provided herein effective to “treat” a disorder in a subject or mammal. In the case of cancer, the therapeutically effective amount of the drug may either reduce the number of cancer cells, reduce the tumor size, inhibit cancer cell infiltration into peripheral organs, inhibit tumor metastasis, inhibit tumor growth to certain extent, lengthen progression-free survival, lengthen overall survival, lengthen time to recurrence after a complete response and/or relieve one or more of the symptoms associated with the cancer to some extent. In some embodiments, response to treatment is evaluated according to RECIST criteria.

Administration “in combination with” one or more further therapeutic agents includes simultaneous (concurrent) and consecutive administration in any order. As used herein, the term “pharmaceutical combination” refers to a product obtained from mixing or combining active ingredients, and includes both fixed and non-fixed combinations of the active ingredients. The term “fixed combination” means that the active ingredients, e.g. a compound of Formula (1) and a co-agent, are both administered to a patient simultaneously in the form of a single entity or dosage. The term “non-fixed combination” means that the active ingredients, e.g. a compound of Formula (1) and a co-agent, are both administered to a patient as separate entities either simultaneously, concurrently or sequentially with no specific time limits, wherein such administration provides therapeutically effective levels of the active ingredients in the body of the patient. The latter also applies to cocktail therapy, e.g. the administration of three or more active ingredients.

In some embodiments, the diseases condition is tumor or cancer. In some embodiments, the cancer or tumor is selected from stomach, colon, rectal, liver, pancreatic, lung, breast, cervix uteri, corpus uteri, ovary, testis, bladder, renal, brain/CNS, head and neck, throat, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer or lymphoma.

In some embodiments, the disease condition comprises abnormal cell proliferation, such as a pre-cancerous lesion.

The present embodiments are particularly useful for the treatment of cancer and for the inhibition of the multiplication of a tumor cell or cancer cell in an animal. Cancer, or a precancerous condition, includes a tumor, metastasis, or any disease or disorder characterized by uncontrolled cell growth, can be treated or prevented by administration the drug-ligand complex of the present embodiments.

Representative examples of precancerous conditions that may be targeted by the compounds of the present embodiments, include: metaplasia, hyperplysia, dysplasia, colorectal polyps, actinic ketatosis, actinic cheilitis, human papillomaviruses, leukoplakia, lychen planus and Bowen's disease.

Representative examples of cancers or tumors that may be targeted by compounds of the present embodiments include: lung cancer, colon cancer, prostate cancer, lymphoma, melanoma, breast cancer, ovarian cancer, testicular cancer, CNS cancer, renal cancer, kidney cancer, pancreatic cancer, stomach cancer, oral cancer, nasal cancer, cervical cancer and leukemia.

In some embodiments, the abnormal proliferation is of cancer cells.

In some embodiments, the cancer is selected from the group consisting of: breast cancer, colorectal cancer, diffuse large B-cell lymphoma, endometrial cancer, follicular lymphoma, gastric cancer, glioblastoma, head and neck cancer, hepatocellular cancer, lung cancer, melanoma, multiple myeloma, ovarian cancer, pancreatic cancer, prostate cancer, and renal cell carcinoma.

In some embodiments, the cancer that is treated is selected from the group consisting of: Acute myeloid leukemia (AML), Breast cancer, Chronic lymphocytic leukemia (CLL), Chronic myelogenous leukemia (CML), Hodgkin lymphoma, Multiple myeloma, Mycosis fungoides, Neuroblastoma, Non-Hodgkin lymphoma (NHL), and Ovarian cancer.

In some embodiments, the present disclosure provides a compound for use in killing a cell. The compound is administered to the cell in an amount sufficient to kill said cell. In an exemplary embodiment, the compound is administered to a subject bearing the cell. In a further exemplary embodiment, the administration serves to retard or stop the growth of a tumor that includes the cell (e.g., the cell can be a tumor cell). For the administration to retard the growth, the rate of growth of the cell should be at least 10% less than the rate of growth before administration. Preferably, the rate of growth will be retarded at least 20%, 30%, 40%, 50%, 60%, 70%, 80%, 90%, or completely stopped.

Additionally, the present disclosure provides a compound or a pharmaceutical composition of the present embodiments for use as a medicament. The present disclosure also provides a compound or a pharmaceutical composition for killing, inhibiting or delaying proliferation of a tumor or cancer cell, or for treating a disease wherein TLR7 and/or TLR8 are implicated.

### Effective Dosages

Pharmaceutical compositions suitable for use with the present embodiments include compositions wherein the active ingredient is contained in a therapeutically effective amount, i.e., in an amount effective to achieve its intended purpose. The actual amount effective for a particular application will depend, inter alia, on the condition being treated. Determination of an effective amount is well within the capabilities of those skilled in the art, especially in light of the detailed disclosure herein.

For any compound described herein, the therapeutically effective amount can be initially determined from cell culture assays. Target plasma concentrations will be those concentrations of active compound(s) that are capable of inhibition cell growth or division. In preferred embodiments, the cellular activity is at least 25% inhibited. Target plasma concentrations of active compound(s) that are capable of inducing at least about 30%, 50%, 75%, or even 90% or higher inhibition of cellular activity are presently preferred. The percentage of inhibition of cellular activity in the patient can be monitored to assess the appropriateness of the plasma drug concentration achieved, and the dosage can be adjusted upwards or downwards to achieve the desired percentage of inhibition.

Therapeutically effective amounts for use in humans can also be determined from animal models. For example, a dose for humans can be formulated to achieve a circulating concentration that has been found to be effective in animals. The dosage in humans can be adjusted by monitoring cellular inhibition and adjusting the dosage upwards or downwards, as described above.

A therapeutically effective dose can also be determined from human data for compounds which are known to exhibit similar pharmacological activities. The applied dose can be adjusted based on the relative bioavailability and potency of the administered compound as compared with the known compound.

Adjusting the dose to achieve maximal efficacy in humans based on the methods described above and other methods as are well-known in the art.

In some embodiments, the composition of the present embodiments is delivered local or regional to a tumor located in the subject, delivered systemically, or delivered via intratumoral injection or by direct injection into tumor vasculature.

In some embodiments, the combination provided herein is formulated for systematic delivery.

In some embodiments, the combination is formulated for oral administration or parenteral injection. In some embodiments, the combination is formulated for intravenous injection or intratumoral injection.

In another aspect, the present disclosure provides a method for treating tumor or abnormal cell proliferation, in a subject that is in need of such treatment, comprising administering to the subject the combination provided herein.

In some embodiments, the method provided herein comprises administering to the subject an oral formulation comprising said immunotherapeutic in a dose of from about 0.0005 mg/kg, 0.0006 mg/mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, or 0.01 mg/kg, to about 0.02 mg/kg, or any range bound by a pair of values in the preceding list, all inclusive, twice per week.

In some embodiments, the method provided herein comprises administering to the subject an oral formulation comprising said immunotherapeutic in a dose of at least 0.0001 mg/kg but less than or about 0.0005 mg/kg, 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, or 0.01 mg/kg, twice per week.

In some embodiments, the method provided herein comprises administering to the subject an intravenous formulation comprising said immunotherapeutic in a dose of from about 0.0005 mg/kg, 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, or 0.006 mg/kg to about 0.015 mg/kg, or any range bound by a pair of values in the preceding list, all inclusive, weekly.

In some embodiments, the method provided herein comprises administering to the subject an intravenous formulation comprising said immune modulatory chemotherapeutic in a dose of about 40-50 mg/kg in divided dose over 2-5 days.

In some embodiments, the combination is adminstered repeatedly at intervals of 2-4 weeks.

In some embodiments, the method provided herein comprises administering to the subject an intravenous formulation comprising said immune modulatory chemotherapeutic in a dose of about 10 to 15 mg/kg, given every 7 to 10 days.

In some embodiments, the method provided herein comprises administering to the subject an intravenous formulation comprising said immune modulatory chemotherapeutic in a dose of about 3 to 5 mg/kg, twice weekly.

In some embodiments, the method provided herein comprises administering to the subject an intravenous formulation comprising said immune modulatory chemotherapeutic in a dose of about 60-120 mg/m²/day, continuous daily.

In some embodiments, the method provided herein comprises administering to the subject an oral formulation comprising said immune modulatory chemotherapeutic in a dose of about 400-1000 mg/m² divided over 4-5 days.

In some embodiments, the method provided herein comprises administering to the subject an intravenous formulation comprising said immune modulatory chemotherapeutic in a dose of about 50-100 mg/m²/day, or 1-5 mg/kg/day.

In some embodiments, the method provided herein comprises administering to the subject an intravenous formulation in the form of intermittent therapy, wherein 40-50 mg/kg in divided dose is administered over 2-5 days. The administration may be repeated at intervals of 2-4 week. In some embodiments, the dose is 10 to 15 mg/kg given every 7 to 10 days; or 3 to 5 mg/kg twice weekly.

In some embodiments, the method provided herein comprises administering to the subject an intravenous formulation in the form of continuous daily therapy, with a dose of 1-2.5 mg/kg/day.

In some embodiments, the method provided herein comprises administering to the subject an oral formulation in the form of intermittent therapy, wherein a dose of 40-50 mg/kg divided to be administered over 4-5 days.

In some embodiments, the method provided herein comprises administering to the subject an oral formulation in the form of continuous daily therapy with a dose of 1-5 mg/kg/day.

In the case of local administration, the systemic circulating concentration of administered compound will not be of particular importance. In such instances, the compound is administered so as to achieve a concentration at the local area effective to achieve the intended result.

Therapeutic amounts of specific antibodies disclosed herein can also be administered, as a component of the combination, with the immunotherapeutics, either in a single mixture form, or separately. In some embodiments, therapeutic amounts are amounts which eliminate or reduce the patient's tumor burden, or which prevent or reduce the proliferation of metastatic cells. The dosage will depend on many parameters, including the nature of the tumor, patient history, patient condition, the possible co-use of other oncolytic agents, and methods of administration. Methods of administration include injection (e.g., parenteral, subcutaneous, intravenous, intraperitoneal, etc.) for which the antibodies are provided in a nontoxic pharmaceutically acceptable carrier such as water, saline, Ringer's solution, dextrose solution, 5% human serum albumin, fixed oils, ethyl oleate, or liposomes. Typical dosages may range from about 0.01 to about 20 mg/kg, such as from about 0.1 to about 10 mg/kg. Other effective methods of administration and dosages may be determined by routine experimentation and are within the scope of this invention.

For other modes of administration, dosage amount and interval can be adjusted individually to provide plasma levels of the administered compound effective for the particular clinical indication being treated. For example, in one embodiment, a compound according to the present embodiments can be administered in relatively high concentrations multiple times per day. Alternatively, it may be more desirable to administer a compound of the present embodiments at minimal effective concentrations and to use a less frequent administration regimen. This will provide a therapeutic regimen that is commensurate with the severity of the individual's disease.

Utilizing the teachings provided herein, an effective therapeutic treatment regimen can be planned which does not cause toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent. In various aspects of these embodiments the toxicity avoided is an observable toxicity, a substantial toxicity, a severe toxicity, or an acceptable toxicity, or a dose-limiting toxicity (such as but not limited to a MTD). By an observable toxicity it is meant that while a change is observed the effect is negligible or mild. By substantial toxicity it is meant that there is a negative impact on the patient's overall health or quality of life. In some instances a substantial toxicity may be mitigated or resolved with other ongoing medical intervention. By a severe toxicity it is meant that the effect requires acute medical intervention and/or dose reduction or suspension of treatment. The acceptability of the toxicity will be influenced by the particular disease being treated and it severity and the availability of mitigating ongoing medical intervention. Toxicities and adverse events are sometimes graded according to a 5 point scale. A grade 1 or mild toxicity is asymptomatic or induces only mild symptoms; may be characterized by clinical or diagnostic observations only; and intervention is not indicated. A grade 2 or moderate toxicity may impair activities of daily living (such as preparing meals, shopping, managing money, using the telephone, etc.) but only minimal, local, or non-invasive interventions are indicated. Grade 3 toxicities are medically significant but not immediately life-threatening; hospitalization or prolongation of hospitalization is indicated; activities of daily living related to self-care (such as bathing, dressing and undressing, feeding oneself, using the toilet, taking medications, and not being bedridden) may be impaired. Grade 4 toxicities are life-threatening and urgent intervention is indicated. Grade 5 toxicity produces an adverse event-related death. Thus in various embodiments, the disclosed therapeutic treatment regimens reduce the grade of a toxicity associated with treatment by at least one grade as compared to a similarly effective dose (if one can be attained) for either active component (the immunotherapeutic or the immune modulating chemotherapeutic) used alone. Alternatively, in various embodiments regimens using the disclosed combinations achieve a therapeutic effect while producing a lesser grade of toxicity than associated with the dose limiting toxicity or one or the other of the required active components of the regiment In other embodiments use of regimens using the disclosed combinations confines a toxicity to grade 2 or less, to grade 1 or less, or produces no observation of the toxicity.

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the present embodiments described herein may be employed in practicing the invention. It is intended that the following claims define the scope of present embodiments and that methods and structures within the scope of these claims and their equivalents be covered thereby.

### EXAMPLES

The present invention is further exemplified, but not limited, by the following and Examples that illustrate the preparation of the compounds of the present embodiments.

### Example 1

In Vivo Tumor Cell Killing Using Cyclophosphamide with TLRL Combination Therapy in Immune Competent Mice

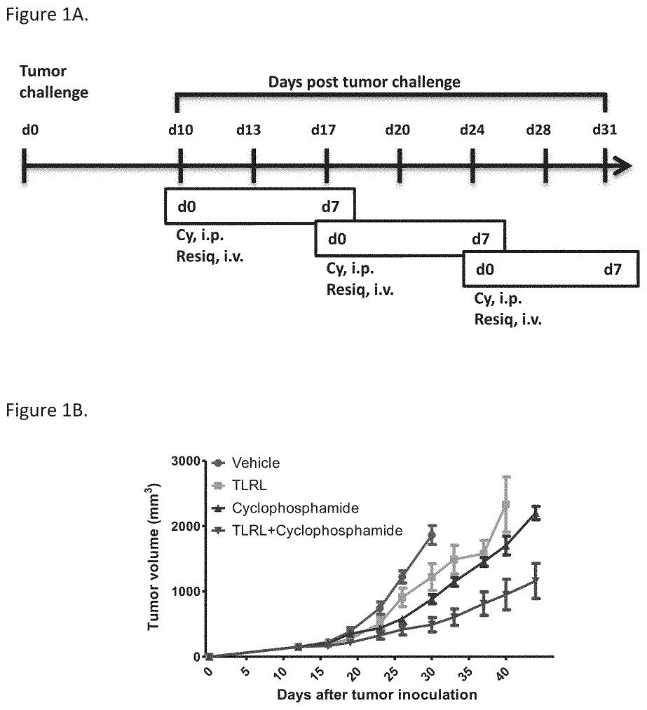

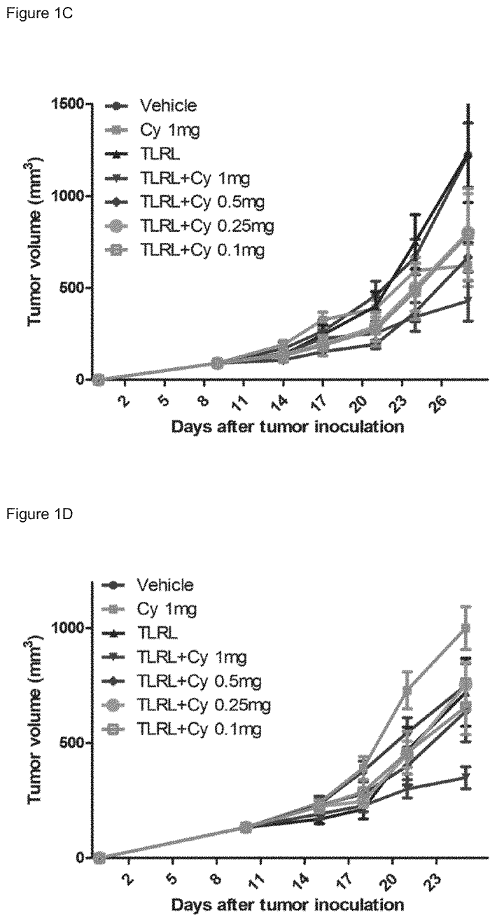

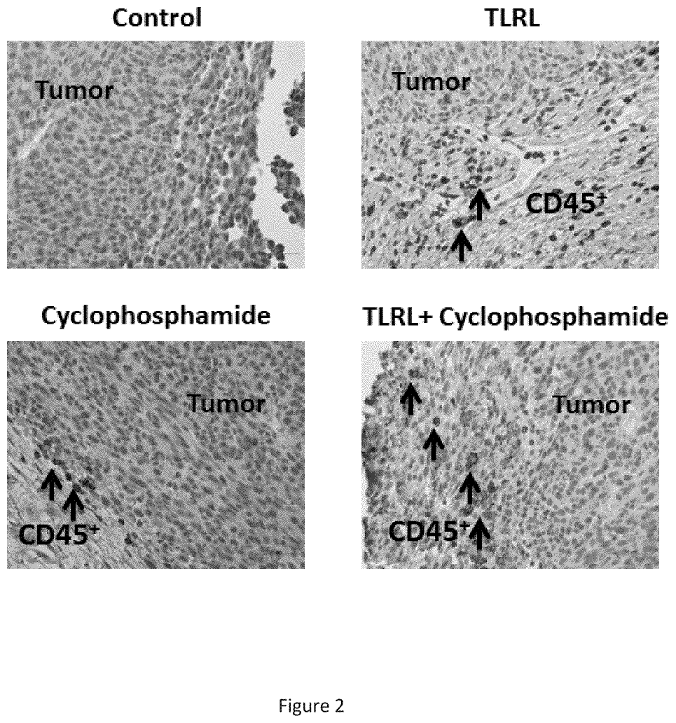

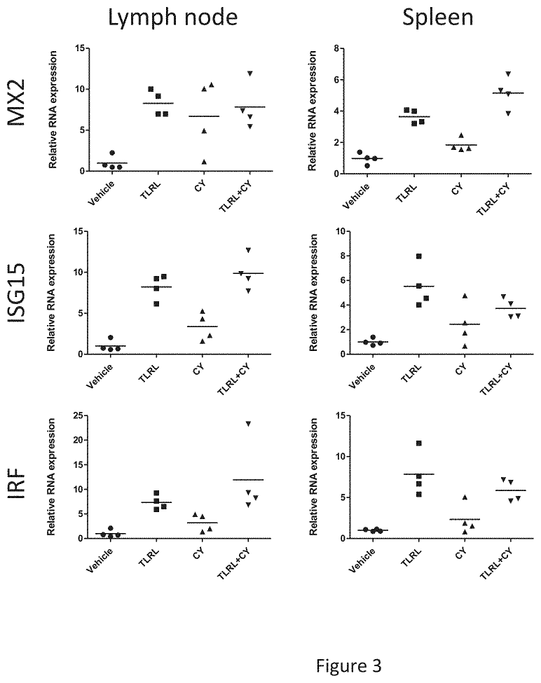

All BALB/c mice were maintained under specific pathogen free conditions and used between 6-16 weeks of age in accordance to the animal experimental guidelines set by the Institutional Animal Care and Use Committee. All experiments have been approved by the Institutional Animal Care and Use Committee and conform to the relevant regulatory standards. C26 colon tumor cell line was cultured in 5% CO₂, and maintained in vitro in RPMI-1640 supplemented with 10% heat-inactivated fetal bovine serum (FBS) (Sigma), 100 units/mL penicillin, and 100 g/mL streptomycin. C26 cells were injected s.c. in the back of 6 to 8-week-old anesthetized mice. Tumor volumes were measured along three orthogonal axes (x, y, and z) and calculated as tumor volume=(xyz)/2. Mice were treated with i.p. injections of cyclophosphamide, i.v. injections of 1.6 g of TLRL (resiquimod), or a combination of both treatments, administered once weekly for three times. Injection time points are indicated in FIG. 1A. Observed tumor volumes are shown in FIG. 1B. The combined treatment had greater effect in reducing tumor volume than either of the individual treatments, which were similar.

In a separate experiment, mice were treated with i.v. injections of 1.6 g of of TLRL (resiquimod) or combination therapy with 1 mg, 0.5 mg, 0.25 mg, or 0.1 mg of cyclophosphamide shown in FIG. 1C.

In a separate experiment, mice were treated with i.v. injections of 3.2 ug of of TLRL (resiquimod) or combination therapy with 1 mg, 0.5 mg, 0.25 mg, or 0.1 mg of cyclophosphamide shown in FIG. 1D.

### Example 2

Isolation of Tumor and Immunohistochemistry

Balb/c mice, 6-8 weeks of age, female, purchased from Vital River Beijing, C26 cells were injected s.c. in the back of anesthetized mice. Treatment was started when tumor volumes reached 100-120 mm³, i.p. injections of cyclophosphamide or i.v. injections of 1.6 g of TLRL (resiquimod) or combination were given once weekly. On second day of second treatment, tissue specimens of the tumor from treated mice were surgically removed, fixed in 4% buffered formaldehyde for 24 h, processed into paraffin, then sectioned at 5 μm. For immunohistochemistry, tissue sections were deparaffinised followed by antigen-retrieval in Tris-EDTA buffer (0.01 M pH 9.0) at high temperature (water bath, 30 minutes at 98° C.). After blocking for non-specific binding with 10% goat serum in PBS, primary antibodies rat anti-mouse CD45 (Purified clone #30-F11, eBioscience™, Thermofisher, Catalog #:14-0451-81) were applied at 1 μg/mL concentrations and incubated for overnight at 4 degree. After washing with PBS three times, secondary antibody goat antibody rat HPR was applied. Standard DAB (3,3′-diaminobenzidine) kit was used for visualization and section was counterstained with H&E used according to the protocols recommended by the manufacturer. A significant numbers of CD45⁺ cell were infiltrated in TLRL (resiquimod) or resiquimod/Cyclophosphamide treated tumor microenvironment.

### Example 3

Detection of Systemic Immune Activation with IFNα Inducible Genes Expression in Mouse Lymph Node and Spleen Treated with Cyclophosphamide, or TLRL or Combination Therapy.

Balb/c mice, 6-8 weeks of age, female, purchased from Vital River Beijing, C26 cells were injected s.c. in the back of anesthetized mice. Treatment was started when tumor volumes reached 100-120 mm³, i.p. injections of cyclophosphamide or i.v. injections of 1.6 g of TLRL (resiquimod) or a combination of the treatments were given once weekly. On second day of second treatment, (that is, on the eighth day) draining LNs and spleen from treated mice were dissected and RNA was extracted using TRIzol reagent. After cDNA was reversed-transcribed, the quantitative Real-Time PCR was performed and IFNα inducible gene expression data were normalized relative to geometric mean of two housekeeping genes (Actin):

(SEQ ID NO.: 1)
Mouse Actin: F: CATTGCTGACAGGATGCAGAAGG
(SEQ ID NO.: 2)
Mouse Actin R: TGCTGGAAGGTGGACAGTGAGG
(SEQ ID NO.: 3)
Mouse Mx2 F: CCTGCCTGCCATCGCTGTC
(SEQ ID NO.: 4)
Mouse Mx2 R: GCCTCTCCACTCCTCTCCCTCATT
(SEQ ID NO.: 5)
Mouse IRF7 F: TTGGGCAAGACTTGTCAGCA
(SEQ ID NO. : 6)
Mouse IRF7 R: ATACCCATGGCTCCAGCTTC
(SEQ ID NO.: 7)
Mouse ISG15 F: CAGCAATGGCCTGGGACCTAA
(SEQ ID NO.: 8)
Mouse ISG15 R: GGAAAGCCGGCACACCAATC.

Upregulating IFNα inducible gene clusters were observed in TLRL (resiquimod) or resiquimod/Cyclophosphamide treated C26 mice.

1. A combination, comprising:
(i) an effective amount of an immune modulatory chemotherapeutic; and
(ii) an effective amount of immunotherapeutic comprising a TLR7 and/or TLR8 agonist activity.

2. The combination of claim 1, where said immunotherapeutic has a structure of Formula (I): 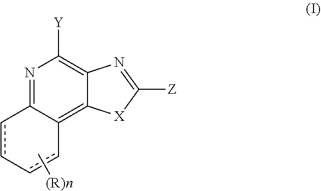
wherein dashed line represents bond or absence of bond;
X is S or —NR₁, R₁ is —W₀—W₁—W₂—W₃—W₄,
W₀ is a bond, alkyl, alkenyl, alkynyl, alkoxy, or -alkyl-S-alkyl-,
W₁ is a bond, —O—, or —NR₂—, wherein R₂ is hydrogen, alkyl or alkenyl,
W₂ is a bond, —O—, —C(O)—, —C(S)—, or —S(O)₂,
W₃ is a bond, —NR₃—, wherein R₃ is hydrogen, alkyl or alkenyl,
W₄ is hydrogen, alkyl, alkenyl, alkynyl, alkoxy, cycloalkyl, aryl, aryloxy, heteroaryl, or heterocyclyl, each of which is optionally substituted by one or more substituents selected from the group consisting of hydroxyl, alkoxy, alkyl, alkenyl, alkynyl, cycloalkyl, aryl, heteroaryl, heterocyclyl, —NH₂, nitro,
-alkyl-hydroxyl, -alkyl-aryl, -alkyl-heteroaryl, -alkyl-heterocyclyl, —O—R₄, —O-alkyl-R₄, -alkyl-O—R₄,
—C(O)—R₄, -alkyl-C(O)—R₄, -alkyl-C(O)—O—R₄, —C(O)—O—R₄, —S—R₄, —S(O)₂—R₄, —NH—S(O)₂—R₄, -alkyl-S—R₄, -alkyl-S(O)₂—R₄, —NHR₄, —NR₄R₄, —NH-alkyl-R₄, halogen, —CN, —NO₂, and —SH, wherein R₄ is independently hydrogen, alkyl, alkenyl, -alkyl-hydroxyl, aryl, heteroaryl, heterocyclyl, or haloalkyl;
Z is hydrogen, alkyl, alkenyl, alkynyl, alkoxy, aryl, haloalkyl, heteroaryl, heterocyclyl, each of which can be optionally substituted by one or more substituents selected from the group consisting of hydroxyl, alkoxy, alkyl, alkenyl, alkynyl, aryl, heteroaryl, heterocyclyl, halogen, cyano, nitro, —N(R₅)₂,
-alkoxy-alkyl, -alkoxy-alkenyl, —C(O)-alkyl, —C(O)—O-alkyl, —O—C(O)-alkyl, —C(O)—N(R₅)₂, aryl, heteroaryl, —CO-aryl, and —CO-heteroaryl, wherein each R₅ is independently hydrogen, alkyl, haloalkyl,
-alkyl-aryl, or -alkyl-heteroaryl;
R is hydrogen, alkyl, alkoxy, haloalkyl, halogen, aryl, heteroaryl, heterocyclyl, each of which is optionally substituted by one or more substituents selected from the group consisting of hydroxyl, alkoxy, alkyl, alkenyl, alkynyl, cycloalkyl, aryl, heteroaryl, heterocyclyl, —NH₂, nitro,
-alkyl-hydroxyl, -alkyl-aryl,
-alkyl-heteroaryl, -alkyl-heterocyclyl, —O—R₄, —O-alkyl-R₄, -alkyl-O—R₄, —C(O)—R₄, —C(O)—NH—R₄,
—C(O)—NR₄R₄, -alkyl-C(O)—R₄, -alkyl-C(O)—O—R₄, —C(O)—O—R₄, —O—C(O)—R₄, —S—R₄, —C(O)—S—R₄,
—S—C(O)—R₄,
—S(O)₂—R₄, —NH—S(O)₂—R₄, -alkyl-S—R₄, -alkyl-S(O)₂—R₄, —NHR₄, —NR₄R₄, —NH-alkyl-R₄, halogen, —CN, and —SH, wherein R₄ is independently hydrogen, alkyl, alkenyl, alkoxy, -alkyl-hydroxyl, aryl, heteroaryl, heterocyclyl, or haloalkyl;
n is 0, 1, 2, 3, or 4;
Y is —NR₆R₇, —CR₆R₇R₈, or -alkyl-NH₂, each of which can be optionally substituted by one or more substituents selected from the group consisting of hydroxyl, alkoxy, alkyl, alkenyl, alkynyl, —NH₂, halogen, —N(R₅)₂, -alkoxy-alkyl, -alkoxy-alkenyl, —C(O)-alkyl, —C(O)—O-alkyl, —C(O)—N(R₅)₂, aryl, heteroaryl,
—CO-aryl, and —CO-heteroaryl,
wherein R₆, R₇ and R₈ are independently hydrogen, alkyl, alkenyl, alkoxy, alkylamino, dialkylamino, alkylthio, arylthio, -alkyl-hydroxyl, -alkyl-C(O)—O—R₉, -alkyl-C(O)—R₉, or -alkyl-O—C(O)—R₉, wherein each R₅ is independently hydrogen, alkyl, haloalkyl, -alkyl-aryl, or -alkyl-heteroaryl, wherein R₉ is hydrogen, alkyl, alkenyl, halogen, or haloalkyl;
X and Z taken together may optionally form a (5-9)-membered ring.

3. The combination of claim 1, wherein said immunotherapeutic is a compound selected from the group consisting of: 2-propylthiazolo[4,5-c]quinolin-4-amine,
1-(2-methylpropyl)-1H-imidazo[4,5-c]quinolin-4-amine,
4-amino-2-(ethoxymethyl)-aa-di-methyl-1H-imidazo[4,5-c]quinoline-1-ethanol,
1-(4-amino-2-ethylaminomethylimidazo-[4,5-c]quinolin-1-yl)-2-methylpropan-2-ol,
N-[4-(4-amino-2-ethyl-1H-imidazo[4,5-c]quinolin-1-yl)butyl-]methanesulfonamide,
4-amino-2-ethoxymethyl-aa-dimethyl-6,7,8,9-tetrahydro-1H-imidazo[4,5-c]quinoline-1-ethanol,
4-amino-aa-dimethyl-2-methoxyethyl-1H-imidazo[4,5-c]quinoline-1-ethanol,
1-{2-[3-(benzyloxy)propoxy]ethyl}-2-(ethoxymethyl)-1H-imidazo[4,5-c]quinolin-4-amine,
1-(2-amino-2-methylpropyl)-2-(ethoxymethyl)-1H-imidazo[4,5-c]quinolin-4-amine,
1-{4-[(3,5-dichlorophenyl)sulfonyl]butyl}-2-ethyl-1H-imidazo[4,5-c]quinolin-4-amine,
N-{3-[4-amino-2-(ethoxymethyl)-1H-imidazo[4,5-c]quinolin-1-yl]propyl}-N′-(3-cyanophenyl)thiourea,
N-[3-(4-amino-2-butyl-1H-imidazo[4,5-c]quinolin-1-yl)-2,2-dimethylpropyl]benzamide,
2-butyl-1-[3-(methylsulfonyl)propyl]-1H-imidazo[4,5-c]quinolin-4-amine,
N-{2-[4-amino-2-(ethoxymethyl)-1H-imidazo[4,5-c]quinolin-1-yl]-1,1-dimethylethyl}-2-ethoxyacetamide,
1-[4-amino-2-ethoxymethyl-7-(pyridin-4-yl)-1H-imidazo[4,5-c]quinolin-1-yl]-2-methylpropan-2-ol,
1-[4-amino-2-(ethoxymethyl)-7-(pyridin-3-yl)-1H-imidazo[4,5-c]quinolin-1-yl]-2-methylpropan-2-ol,
N-{3-[4-amino-1-(2-hydroxy-2-methylpropyl)-2-(methoxyethyl)-1H-imidazo[4,5-c]quinolin-7-yl]phenyl}methanesulfonamide,
1-[4-amino-7-(5-hydroxymethylpyridin-3-yl)-2-(2-methoxyethyl)-1H-imidazo[4,5-c]quinolin-1-yl]-2-methylpropan-2-ol,
3-[4-amino-2-(ethoxymethyl)-7-(pyridin-3-yl)-1H-imidazo[4,5-c]quinolin-1-yl]propane-1,2-diol,
1-[2-(4-amino-2-ethoxymethyl-1H-imidazo[4,5-c]quinolin-1-yl)-1,1-dimethylethyl]-3-propylurea,
1-[2-(4-amino-2-ethoxymethyl-1H-imidazo[4,5-c]quinolin-1-yl)-1,1-dimethylethyl]-3-cyclopentylurea,
1-[(2,2-dimethyl-1,3-dioxolan-4-yl)methyl]-2-(ethoxymethyl)-7-(4-hydroxymethylphenyl)-1H-imidazo[4,5-c]quinolin-4-amine,
4-[4-amino-2-ethoxymethyl-1-(2-hydroxy-2-methylpropyl)-1H-imidazo[4,5-c]quinolin-7-yl]-N-methoxy-N-methylbenzamide,
2-ethoxymethyl-N1-isopropyl-6,7,8,9-tetrahydro-1H-imidazo[4,5-c]quinoline-1,4-diamine,
1-[4-amino-2-ethyl-7-(pyridin-4-yl)-1H-imidazo[4,5-c]quinolin-1-yl]-2-methylpropan-2-ol, and
N-[4-(4-amino-2-ethyl-1H-imidazo[4,5-c]quinolin-1-yl)butyl]methanesulfonamide.

4. The combination of claim 1, where said immunotherapeutic comprises resiquimod.

5. The combination of claim 1, wherein said immunotherapeutic is of an amount that is capable of:
(1) inducing IFN-α in an enriched human blood DCs;
(2) inducing TNF-α in an enriched human blood DCs; and/or
(3) inducing IL-12-α in an enriched human blood DCs.

6. The combination of claim 1, wherein said immune modulatory chemotherapeutic comprises an anti-tumor agent.

7. The combination of claim 6, wherein said anti-tumor agent is selected from the group consisting of: Anthracyclines, Bortezomib, Oxaliplatin, and Cyclophosphamide.

8. The combination of claim 1, wherein said immune modulatory chemotherapeutic comprises a Treg inhibitor.

9. The combination of claim 8, wherein said Treg inhibitor is selected from the group consisting of: Dasatinib, Cyclophoshamide, Temozolomide, Docetaxel, and 5-Fluorouracile.

10. The combination of claim 1, wherein said immune modulatory chemotherapeutic comprise a myeloid-derived suppressor cells (MDSC) inhibitor.

11. The combination of claim 10, wherein said MDSC inhibitor is selected from the group consisting of: Paclitaxel, Gemcitabine, 5-Fluorouracile, Oxaliplatin, Cisplatin, Carboplatin, Dasatinib, Sunitinib, and Doxorubicin.

12. The combination of claim 1, wherein said immune modulatory chemotherapeutic comprise an NK cell activator.

13. The combination of claim 12, wherein said NK cell activator is selected from the group consisting of: Dasatinib, and Imatinib.

14. The combination of claim 1, where said combination is formulated for systematic delivery.

15. The combination of claim 1, where said combination is formulated for oral administration or parenteral injection.

16. The combination of claim 1, where said combination is formulated for intravenous injection or intratumoral injection.

17. The combination of claim 1, wherein said immunotherapeutic is an agonist for both TLR7 and TLR8.

18. A method for treating tumor or abnormal cell proliferation, in a subject that is in need of such treatment, comprising administering to said subject the combination of claim 1.

19. The method of claim 18, wherein said abnormal cell proliferation comprises a pre-cancerous lesion.

20. The method of claim 18, wherein said abnormal proliferation is of cancer cells.

21. The method of claim 20, wherein said cancer is selected from the group consisting of: Acute myeloid leukemia (AML), Breast cancer, Chronic lymphocytic leukemia (CLL), Chronic myelogenous leukemia (CML), Hodgkin lymphoma, Multiple myeloma, Mycosis fungoides, Neuroblastoma, Non-Hodgkin lymphoma (NHL), Ovarian cancer, and Retinoblastoma.

22. The method of claim 18, comprising administering to said subject an oral formulation comprising said immunotherapeutic in a dose of from about 0.0005 mg/kg, 0.0006 mg/mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, or 0.01 mg/kg, to about 0.02 mg/kg, all inclusive, twice per week.

23. The method of claim 18, comprising administering to said subject an oral formulation comprising said immunotherapeutic in a dose of at least 0.0001 mg/kg but less than or about 0.0005 mg/kg, 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, or 0.01 mg/kg, twice per week.

24. The method of claim 18, comprising administering to said subject an intravenous formulation comprising said immunotherapeutic in a dose of from about 0.0005 mg/kg, 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, or 0.006 mg/kg to about 0.015 mg/kg, all inclusive, weekly.

25. The method of claim 18, comprising administering to said subject an intravenous formulation comprising said immunotherapeutic in a dose of at least 0.0001 mg/kg but less than or about 0.003 mg/kg, 0.004 mg/kg, 0.005 mg/kg, 0.006 mg/kg, or 0.01 mg/kg, weekly.

26. The method of claim 18, wherein said immunotherapeutic in said subject has a local concentration that is between about 0.005 μg/ml and about 12 μg/ml.

27. The method of claim 18, wherein said immunotherapeutic in said subject has a local concentration that is from about 0.05 μg/ml, 0.1 μg/ml, 0.15 μg/ml, 0.2 μg/ml, 0.3 μg/ml, or 0.4 μg/ml, to about 0.5 μg/ml.

28. The method of claim 18, comprising administering to said subject an intravenous formulation comprising said immune modulatory chemotherapeutic in a dose of about 40-50 mg/kg in divided dose over 2-5 days.

29. The method of claim 28, wherein said combination is administered over 1-5 days repeatedly at intervals of 2-4 weeks.

30. The method of claim 18, comprising administering to said subject an intravenous formulation comprising said immune modulatory chemotherapeutic in a dose of about 10 to 15 mg/kg, given every 7 to 10 days.

31. The method of claim 18, comprising administering to said subject an intravenous formulation comprising said immune modulatory chemotherapeutic in a dose of about 3 to 5 mg/kg, twice weekly.

32. The method of claim 18, comprising administering to said subject an intravenous formulation comprising said immune modulatory chemotherapeutic in a dose of about 60-120 mg/m²/day.

33. The method of claim 18, comprising administering to said subject an oral formulation comprising said immune modulatory chemotherapeutic in a dose of about 400-1000 mg/m² divided over 4-5 days.

34. The method of claim 18, comprising administering to said subject an intravenous formulation comprising said immune modulatory chemotherapeutic in a dose of about 50-100 mg/m²/day, or 1-5 mg/kg/day.

35. A kit, comprising the combination of claim 1.


SEQUENCE LISTING (8 sequences)

SEQ ID NO: 1 - DNA, 23 bp, Artificial Sequence
  Mouse actin forward primer
cattgctgacaggatgcagaagg

SEQ ID NO: 2 - DNA, 22 bp, Artificial Sequence
  Mouse actin reverse primer
tgctggaaggtggacagtgagg

SEQ ID NO: 3 - DNA, 19 bp, Artificial Sequence
  Mouse Mx2 forward primer
cctgcctgccatcgctgtc

SEQ ID NO: 4 - DNA, 24 bp, Artificial Sequence
  Mouse Mx2 reverse primer
gcctctccactcctctccctcatt

SEQ ID NO: 5 - DNA, 20 bp, Artificial Sequence
  Mouse IRF7 forward primer
ttgggcaagacttgtcagca

SEQ ID NO: 6 - DNA, 20 bp, Artificial Sequence
  Mouse IRF7 reverse primer
atacccatggctccagcttc

SEQ ID NO: 7 - DNA, 21 bp, Artificial Sequence
  Mouse ISG15 forward primer
cagcaatggcctgggacctaa

SEQ ID NO: 8 - DNA, 20 bp, Artificial Sequence
  Mouse ISG15 reverse primer
ggaaagccggcacaccaatc